SEQUENCE LISTING (402 sequences)

SEQ ID NO: 1 - protein, 8 aa, Artificial sequence
  Synthetic antibody
GYTFTNSY

SEQ ID NO: 2 - protein, 8 aa, Artificial sequence
  Synthetic antibody
INPISGGT

SEQ ID NO: 3 - protein, 20 aa, Artificial sequence
  Synthetic antibody
ARDRGDYDYGWGTSPFYFDY

SEQ ID NO: 4 - protein, 6 aa, Artificial sequence
  Synthetic antibody
QSISRY

SEQ ID NO: 5 - protein, 3 aa, Artificial sequence
  Synthetic antibody
AAS

SEQ ID NO: 6 - protein, 9 aa, Artificial sequence
  Synthetic antibody
QQSFSPPIT

SEQ ID NO: 7 - protein, 8 aa, Artificial sequence
  Synthetic antibody
GYTFTNSY

SEQ ID NO: 8 - protein, 8 aa, Artificial sequence
  Synthetic antibody
INPISGGT

SEQ ID NO: 9 - protein, 20 aa, Artificial sequence
  Synthetic antibody
ARDRGDYDYGWGTSPFYFDY

SEQ ID NO: 10 - protein, 6 aa, Artificial sequence
  Synthetic antibody
QPISKY

SEQ ID NO: 11 - protein, 3 aa, Artificial sequence
  Synthetic antibody
AAS

SEQ ID NO: 12 - protein, 9 aa, Artificial sequence
  Synthetic antibody
QQSNGIPLT

SEQ ID NO: 13 - protein, 8 aa, Artificial sequence
  Synthetic antibody
GYTFTNSY

SEQ ID NO: 14 - protein, 8 aa, Artificial sequence
  Synthetic antibody
INPISGGT

SEQ ID NO: 15 - protein, 20 aa, Artificial sequence
  Synthetic antibody
ARDRGDYDYGWGTSPFYFDY

SEQ ID NO: 16 - protein, 6 aa, Artificial sequence
  Synthetic antibody
QSISSY

SEQ ID NO: 17 - protein, 3 aa, Artificial sequence
  Synthetic antibody
AAS

SEQ ID NO: 18 - protein, 9 aa, Artificial sequence
  Synthetic antibody
QQSYSTPLT

SEQ ID NO: 19 - protein, 8 aa, Artificial sequence
  Synthetic antibody
GGSFSGYY

SEQ ID NO: 20 - protein, 7 aa, Artificial sequence
  Synthetic antibody
INHGGST

SEQ ID NO: 21 - protein, 18 aa, Artificial sequence
  Synthetic antibody
ARGYDTNWYGDGYNWFDP

SEQ ID NO: 22 - protein, 6 aa, Artificial sequence
  Synthetic antibody
QSISRY

SEQ ID NO: 23 - protein, 3 aa, Artificial sequence
  Synthetic antibody
AAS

SEQ ID NO: 24 - protein, 9 aa, Artificial sequence
  Synthetic antibody
QQSFGTPLT

SEQ ID NO: 25 - protein, 8 aa, Artificial sequence
  Synthetic antibody
GFTFDDYG

SEQ ID NO: 26 - protein, 8 aa, Artificial sequence
  Synthetic antibody
INWNGERI

SEQ ID NO: 27 - protein, 14 aa, Artificial sequence
  Synthetic antibody
ARPSGDYVAWYFNL

SEQ ID NO: 28 - protein, 9 aa, Artificial sequence
  Synthetic antibody
GSDVGPYKY

SEQ ID NO: 29 - protein, 3 aa, Artificial sequence
  Synthetic antibody
DVN

SEQ ID NO: 30 - protein, 10 aa, Artificial sequence
  Synthetic antibody
GSYAGNNKWV

SEQ ID NO: 31 - protein, 9 aa, Artificial sequence
  Synthetic antibody
GGSISSNNW

SEQ ID NO: 32 - protein, 7 aa, Artificial sequence
  Synthetic antibody
IHHSGGT

SEQ ID NO: 33 - protein, 14 aa, Artificial sequence
  Synthetic antibody
TRDRAGGTYSGFDF

SEQ ID NO: 34 - protein, 9 aa, Artificial sequence
  Synthetic antibody
SSDVGGYNY

SEQ ID NO: 35 - protein, 3 aa, Artificial sequence
  Synthetic antibody
DVS

SEQ ID NO: 36 - protein, 10 aa, Artificial sequence
  Synthetic antibody
SSYTSSSTVV

SEQ ID NO: 37 - protein, 8 aa, Artificial sequence
  Synthetic antibody
GFTFSDFG

SEQ ID NO: 38 - protein, 8 aa, Artificial sequence
  Synthetic antibody
TSHDGSSK

SEQ ID NO: 39 - protein, 15 aa, Artificial sequence
  Synthetic antibody
AKDSDNGYDADFFDY

SEQ ID NO: 40 - protein, 6 aa, Artificial sequence
  Synthetic antibody
QSISSY

SEQ ID NO: 41 - protein, 3 aa, Artificial sequence
  Synthetic antibody
AAS

SEQ ID NO: 42 - protein, 9 aa, Artificial sequence
  Synthetic antibody
QQSYSTPLT

SEQ ID NO: 43 - protein, 8 aa, Artificial sequence
  Synthetic antibody
GGSFSGYY

SEQ ID NO: 44 - protein, 7 aa, Artificial sequence
  Synthetic antibody
INHGGST

SEQ ID NO: 45 - protein, 18 aa, Artificial sequence
  Synthetic antibody
ARGYDTNWYGDGYNWFDP

SEQ ID NO: 46 - protein, 6 aa, Artificial sequence
  Synthetic antibody
SLRGSF

SEQ ID NO: 47 - protein, 3 aa, Artificial sequence
  Synthetic antibody
GIN

SEQ ID NO: 48 - protein, 11 aa, Artificial sequence
  Synthetic antibody
NSRESNSNRIL

SEQ ID NO: 49 - protein, 8 aa, Artificial sequence
  Synthetic antibody
GFSFDNYA

SEQ ID NO: 50 - protein, 8 aa, Artificial sequence
  Synthetic antibody
ITGNSGTI

SEQ ID NO: 51 - protein, 15 aa, Artificial sequence
  Synthetic antibody
AKDTDYDSSGSYFDY

SEQ ID NO: 52 - protein, 6 aa, Artificial sequence
  Synthetic antibody
NIGRES

SEQ ID NO: 53 - protein, 3 aa, Artificial sequence
  Synthetic antibody
SDG

SEQ ID NO: 54 - protein, 11 aa, Artificial sequence
  Synthetic antibody
QVWDPDTDHYV

SEQ ID NO: 55 - protein, 8 aa, Artificial sequence
  Synthetic antibody
GFTFSSYA

SEQ ID NO: 56 - protein, 8 aa, Artificial sequence
  Synthetic antibody
VSDDGNMK

SEQ ID NO: 57 - protein, 16 aa, Artificial sequence
  Synthetic antibody
ARENYFWSGSIGGLDY

SEQ ID NO: 58 - protein, 6 aa, Artificial sequence
  Synthetic antibody
QSISSY

SEQ ID NO: 59 - protein, 3 aa, Artificial sequence
  Synthetic antibody
AAS

SEQ ID NO: 60 - protein, 10 aa, Artificial sequence
  Synthetic antibody
QQSYSTPGYT

SEQ ID NO: 61 - protein, 8 aa, Artificial sequence
  Synthetic antibody
GGNFRSHT

SEQ ID NO: 62 - protein, 8 aa, Artificial sequence
  Synthetic antibody
IMPRFGAT

SEQ ID NO: 63 - protein, 12 aa, Artificial sequence
  Synthetic antibody
AADLGSGRKFDS

SEQ ID NO: 64 - protein, 6 aa, Artificial sequence
  Synthetic antibody
QGIGSD

SEQ ID NO: 65 - protein, 3 aa, Artificial sequence
  Synthetic antibody
AAS

SEQ ID NO: 66 - protein, 9 aa, Artificial sequence
  Synthetic antibody
LQHNSYPLT

SEQ ID NO: 67 - protein, 10 aa, Artificial sequence
  Synthetic antibody
GDSVSSNSAA

SEQ ID NO: 68 - protein, 9 aa, Artificial sequence
  Synthetic antibody
TYYRSKWYN

SEQ ID NO: 69 - protein, 18 aa, Artificial sequence
  Synthetic antibody
AREEQQLVHDYYYYGMDV

SEQ ID NO: 70 - protein, 9 aa, Artificial sequence
  Synthetic antibody
SSDIGAYNY

SEQ ID NO: 71 - protein, 3 aa, Artificial sequence
  Synthetic antibody
EVS

SEQ ID NO: 72 - protein, 8 aa, Artificial sequence
  Synthetic antibody
SSYAGSIS

SEQ ID NO: 73 - protein, 10 aa, Artificial sequence
  Synthetic antibody
GDSVSSNSAA

SEQ ID NO: 74 - protein, 9 aa, Artificial sequence
  Synthetic antibody
TYYRSKWYN

SEQ ID NO: 75 - protein, 18 aa, Artificial sequence
  Synthetic antibody
AREEQQLVHDYYYYGMDV

SEQ ID NO: 76 - protein, 9 aa, Artificial sequence
  Synthetic antibody
SCTGISSDY

SEQ ID NO: 77 - protein, 3 aa, Artificial sequence
  Synthetic antibody
EVN

SEQ ID NO: 78 - protein, 9 aa, Artificial sequence
  Synthetic antibody
GSYAGSNTF

SEQ ID NO: 79 - protein, 8 aa, Artificial sequence
  Synthetic antibody
GGSFSGYY

SEQ ID NO: 80 - protein, 7 aa, Artificial sequence
  Synthetic antibody
INHGGST

SEQ ID NO: 81 - protein, 18 aa, Artificial sequence
  Synthetic antibody
ARGYDTNWYGDGYNWFDP

SEQ ID NO: 82 - protein, 8 aa, Artificial sequence
  Synthetic antibody
SSSIGSNT

SEQ ID NO: 83 - protein, 3 aa, Artificial sequence
  Synthetic antibody
NNN

SEQ ID NO: 84 - protein, 11 aa, Artificial sequence
  Synthetic antibody
QSYDTGLSGHV

SEQ ID NO: 85 - protein, 8 aa, Artificial sequence
  Synthetic antibody
GFTFSSYS

SEQ ID NO: 86 - protein, 8 aa, Artificial sequence
  Synthetic antibody
ISSSSSYI

SEQ ID NO: 87 - protein, 18 aa, Artificial sequence
  Synthetic antibody
ARGNVDIVATGEVDAFDI

SEQ ID NO: 88 - protein, 8 aa, Artificial sequence
  Synthetic antibody
SSTIGSNY

SEQ ID NO: 89 - protein, 3 aa, Artificial sequence
  Synthetic antibody
RNN

SEQ ID NO: 90 - protein, 11 aa, Artificial sequence
  Synthetic antibody
AAWDDSLSGYV

SEQ ID NO: 91 - protein, 8 aa, Artificial sequence
  Synthetic antibody
GGTFSNYG

SEQ ID NO: 92 - protein, 8 aa, Artificial sequence
  Synthetic antibody
IIPIFGTA

SEQ ID NO: 93 - protein, 19 aa, Artificial sequence
  Synthetic antibody
ARDRDDALTGLGLGGGFDI

SEQ ID NO: 94 - protein, 8 aa, Artificial sequence
  Synthetic antibody
TSNIGTNT

SEQ ID NO: 95 - protein, 3 aa, Artificial sequence
  Synthetic antibody
GND

SEQ ID NO: 96 - protein, 11 aa, Artificial sequence
  Synthetic antibody
AAWDERLNGYV

SEQ ID NO: 97 - protein, 8 aa, Artificial sequence
  Synthetic antibody
GYTFTNSY

SEQ ID NO: 98 - protein, 8 aa, Artificial sequence
  Synthetic antibody
INPISGGT

SEQ ID NO: 99 - protein, 20 aa, Artificial sequence
  Synthetic antibody
ARDRGDYDYGWGTSPFYFDY

SEQ ID NO: 100 - protein, 6 aa, Artificial sequence
  Synthetic antibody
HTVNSY

SEQ ID NO: 101 - protein, 3 aa, Artificial sequence
  Synthetic antibody
AAS

SEQ ID NO: 102 - protein, 9 aa, Artificial sequence
  Synthetic antibody
QQSYRTPLT

SEQ ID NO: 103 - protein, 8 aa, Artificial sequence
  Synthetic antibody
GFIFDDYA

SEQ ID NO: 104 - protein, 8 aa, Artificial sequence
  Synthetic antibody
VNRDSTYV

SEQ ID NO: 105 - protein, 14 aa, Artificial sequence
  Synthetic antibody
VRGMTRVATDAFDF

SEQ ID NO: 106 - protein, 6 aa, Artificial sequence
  Synthetic antibody
QSINGY

SEQ ID NO: 107 - protein, 3 aa, Artificial sequence
  Synthetic antibody
SAS

SEQ ID NO: 108 - protein, 10 aa, Artificial sequence
  Synthetic antibody
QQSYSTPPVT

SEQ ID NO: 109 - protein, 8 aa, Artificial sequence
  Synthetic antibody
GYTFTNSY

SEQ ID NO: 110 - protein, 8 aa, Artificial sequence
  Synthetic antibody
INPISGGT

SEQ ID NO: 111 - protein, 20 aa, Artificial sequence
  Synthetic antibody
ARDRGDYDYGWGTSPFYFDY

SEQ ID NO: 112 - protein, 6 aa, Artificial sequence
  Synthetic antibody
QSISSY

SEQ ID NO: 113 - protein, 3 aa, Artificial sequence
  Synthetic antibody
GAS

SEQ ID NO: 114 - protein, 9 aa, Artificial sequence
  Synthetic antibody
QQSYSIPFT

SEQ ID NO: 115 - protein, 8 aa, Artificial sequence
  Synthetic antibody
GFTFDDYA

SEQ ID NO: 116 - protein, 8 aa, Artificial sequence
  Synthetic antibody
ISWNSGSI

SEQ ID NO: 117 - protein, 14 aa, Artificial sequence
  Synthetic antibody
AKDMDIWFGGGLDY

SEQ ID NO: 118 - protein, 6 aa, Artificial sequence
  Synthetic antibody
EGIGNW

SEQ ID NO: 119 - protein, 3 aa, Artificial sequence
  Synthetic antibody
EAS

SEQ ID NO: 120 - protein, 9 aa, Artificial sequence
  Synthetic antibody
LQANSFPIT

SEQ ID NO: 121 - protein, 8 aa, Artificial sequence
  Synthetic antibody
GGSFSGYY

SEQ ID NO: 122 - protein, 7 aa, Artificial sequence
  Synthetic antibody
INHGGST

SEQ ID NO: 123 - protein, 12 aa, Artificial sequence
  Synthetic antibody
ARGLVGGGAFDI

SEQ ID NO: 124 - protein, 6 aa, Artificial sequence
  Synthetic antibody
NIGSTG

SEQ ID NO: 125 - protein, 3 aa, Artificial sequence
  Synthetic antibody
DDT

SEQ ID NO: 126 - protein, 11 aa, Artificial sequence
  Synthetic antibody
QVWDSSGHSYV

SEQ ID NO: 127 - protein, 8 aa, Artificial sequence
  Synthetic antibody
GYTFTGYY

SEQ ID NO: 128 - protein, 8 aa, Artificial sequence
  Synthetic antibody
INPNSGGT

SEQ ID NO: 129 - protein, 7 aa, Artificial sequence
  Synthetic antibody
ARGYFDY

SEQ ID NO: 130 - protein, 8 aa, Artificial sequence
  Synthetic antibody
SSNIGSNT

SEQ ID NO: 131 - protein, 3 aa, Artificial sequence
  Synthetic antibody
SNN

SEQ ID NO: 132 - protein, 11 aa, Artificial sequence
  Synthetic antibody
ASWDDSLNGVV

SEQ ID NO: 133 - protein, 8 aa, Artificial sequence
  Synthetic antibody
GYTFTGYY

SEQ ID NO: 134 - protein, 8 aa, Artificial sequence
  Synthetic antibody
INPNSGGT

SEQ ID NO: 135 - protein, 7 aa, Artificial sequence
  Synthetic antibody
ARGYFDY

SEQ ID NO: 136 - protein, 8 aa, Artificial sequence
  Synthetic antibody
SSNIGSNS

SEQ ID NO: 137 - protein, 3 aa, Artificial sequence
  Synthetic antibody
AND

SEQ ID NO: 138 - protein, 11 aa, Artificial sequence
  Synthetic antibody
AAWDNSLKGVV

SEQ ID NO: 139 - protein, 8 aa, Artificial sequence
  Synthetic antibody
GGSFSGYY

SEQ ID NO: 140 - protein, 7 aa, Artificial sequence
  Synthetic antibody
INHGGST

SEQ ID NO: 141 - protein, 18 aa, Artificial sequence
  Synthetic antibody
ARGYDTNWYGDGYNWFDP

SEQ ID NO: 142 - protein, 8 aa, Artificial sequence
  Synthetic antibody
SSNIGTNP

SEQ ID NO: 143 - protein, 3 aa, Artificial sequence
  Synthetic antibody
YNN

SEQ ID NO: 144 - protein, 11 aa, Artificial sequence
  Synthetic antibody
AAWDDSLKGWV

SEQ ID NO: 145 - protein, 8 aa, Artificial sequence
  Synthetic antibody
GGTFSSYA

SEQ ID NO: 146 - protein, 8 aa, Artificial sequence
  Synthetic antibody
IIPIFGTA

SEQ ID NO: 147 - protein, 15 aa, Artificial sequence
  Synthetic antibody
ARDPYSGSYPGAFDI

SEQ ID NO: 148 - protein, 9 aa, Artificial sequence
  Synthetic antibody
SSDVGGYNY

SEQ ID NO: 149 - protein, 3 aa, Artificial sequence
  Synthetic antibody
DVS

SEQ ID NO: 150 - protein, 10 aa, Artificial sequence
  Synthetic antibody
NSYTRSSTSV

SEQ ID NO: 151 - protein, 8 aa, Artificial sequence
  Synthetic antibody
GFTFSSYW

SEQ ID NO: 152 - protein, 8 aa, Artificial sequence
  Synthetic antibody
IKQDGSEK

SEQ ID NO: 153 - protein, 19 aa, Artificial sequence
  Synthetic antibody
ARMADDDFWRDLPGFYMDV

SEQ ID NO: 154 - protein, 9 aa, Artificial sequence
  Synthetic antibody
SSDVGGYNY

SEQ ID NO: 155 - protein, 3 aa, Artificial sequence
  Synthetic antibody
DVS

SEQ ID NO: 156 - protein, 10 aa, Artificial sequence
  Synthetic antibody
SSYTSSSTLV

SEQ ID NO: 157 - protein, 8 aa, Artificial sequence
  Synthetic antibody
GGSFSGYS

SEQ ID NO: 158 - protein, 7 aa, Artificial sequence
  Synthetic antibody
INHGGST

SEQ ID NO: 159 - protein, 18 aa, Artificial sequence
  Synthetic antibody
ARGYDTNWYGDGYNWFDP

SEQ ID NO: 160 - protein, 6 aa, Artificial sequence
  Synthetic antibody
NIGRKS

SEQ ID NO: 161 - protein, 3 aa, Artificial sequence
  Synthetic antibody
RDN

SEQ ID NO: 162 - protein, 9 aa, Artificial sequence
  Synthetic antibody
QVWDSNTGV

SEQ ID NO: 163 - protein, 8 aa, Artificial sequence
  Synthetic antibody
RFTFDDYA

SEQ ID NO: 164 - protein, 8 aa, Artificial sequence
  Synthetic antibody
SSWNSGTI

SEQ ID NO: 165 - protein, 16 aa, Artificial sequence
  Synthetic antibody
AILPGDYNRVADVFDI

SEQ ID NO: 166 - protein, 9 aa, Artificial sequence
  Synthetic antibody
SSNIGAGYD

SEQ ID NO: 167 - protein, 3 aa, Artificial sequence
  Synthetic antibody
GNN

SEQ ID NO: 168 - protein, 11 aa, Artificial sequence
  Synthetic antibody
QSYDSSLSGYV

SEQ ID NO: 169 - protein, 8 aa, Artificial sequence
  Synthetic antibody
RFTFDDHA

SEQ ID NO: 170 - protein, 8 aa, Artificial sequence
  Synthetic antibody
ISWNGGII

SEQ ID NO: 171 - protein, 16 aa, Artificial sequence
  Synthetic antibody
AKGMGFGESNYYAMDV

SEQ ID NO: 172 - protein, 9 aa, Artificial sequence
  Synthetic antibody
SSDVGGYNY

SEQ ID NO: 173 - protein, 3 aa, Artificial sequence
  Synthetic antibody
DVS

SEQ ID NO: 174 - protein, 11 aa, Artificial sequence
  Synthetic antibody
SSYTSISNLVV

SEQ ID NO: 175 - protein, 8 aa, Artificial sequence
  Synthetic antibody
GGSFSGYS

SEQ ID NO: 176 - protein, 7 aa, Artificial sequence
  Synthetic antibody
VNHGGKT

SEQ ID NO: 177 - protein, 18 aa, Artificial sequence
  Synthetic antibody
ARGYDTNWYGDGYNWFDP

SEQ ID NO: 178 - protein, 6 aa, Artificial sequence
  Synthetic antibody
QKIDNF

SEQ ID NO: 179 - protein, 3 aa, Artificial sequence
  Synthetic antibody
GAS

SEQ ID NO: 180 - protein, 9 aa, Artificial sequence
  Synthetic antibody
QQSYNTPIT

SEQ ID NO: 181 - protein, 8 aa, Artificial sequence
  Synthetic antibody
GGSFSGYS

SEQ ID NO: 182 - protein, 7 aa, Artificial sequence
  Synthetic antibody
INHGGST

SEQ ID NO: 183 - protein, 18 aa, Artificial sequence
  Synthetic antibody
ARGYDTNWYGDGYNWFDP

SEQ ID NO: 184 - protein, 6 aa, Artificial sequence
  Synthetic antibody
NIGTKS

SEQ ID NO: 185 - protein, 3 aa, Artificial sequence
  Synthetic antibody
YDS

SEQ ID NO: 186 - protein, 10 aa, Artificial sequence
  Synthetic antibody
QVGDSSSHVV

SEQ ID NO: 187 - protein, 11 aa, Artificial sequence
  Synthetic antibody
GGSISSNNWWT

SEQ ID NO: 188 - protein, 16 aa, Artificial sequence
  Synthetic antibody
EIHHSGGTNYNPSLKS

SEQ ID NO: 189 - protein, 12 aa, Artificial sequence
  Synthetic antibody
DRAGGTYSGFDF

SEQ ID NO: 190 - protein, 14 aa, Artificial sequence
  Synthetic antibody
TGTSSDVGGYNYVS

SEQ ID NO: 191 - protein, 7 aa, Artificial sequence
  Synthetic antibody
DVSNRPS

SEQ ID NO: 192 - protein, 10 aa, Artificial sequence
  Synthetic antibody
SSYTSSSTVV

SEQ ID NO: 193 - protein, 10 aa, Artificial sequence
  Synthetic antibody
GGSFSGYYWT

SEQ ID NO: 194 - protein, 16 aa, Artificial sequence
  Synthetic antibody
EINHGGSTRYNPSLES

SEQ ID NO: 195 - protein, 16 aa, Artificial sequence
  Synthetic antibody
GYDTNWYGDGYNWFDP

SEQ ID NO: 196 - protein, 11 aa, Artificial sequence
  Synthetic antibody
QGDSLRGSFAS

SEQ ID NO: 197 - protein, 7 aa, Artificial sequence
  Synthetic antibody
GINNRPS

SEQ ID NO: 198 - protein, 11 aa, Artificial sequence
  Synthetic antibody
NSRESNSNRIL

SEQ ID NO: 199 - protein, 10 aa, Artificial sequence
  Synthetic antibody
GFTFSSYAMQ

SEQ ID NO: 200 - protein, 17 aa, Artificial sequence
  Synthetic antibody
VVSDDGNMKFYADSVKG

SEQ ID NO: 201 - protein, 14 aa, Artificial sequence
  Synthetic antibody
ENYFWSGSIGGLDY

SEQ ID NO: 202 - protein, 11 aa, Artificial sequence
  Synthetic antibody
RASQSISSYLN

SEQ ID NO: 203 - protein, 7 aa, Artificial sequence
  Synthetic antibody
AASSLQS

SEQ ID NO: 204 - protein, 10 aa, Artificial sequence
  Synthetic antibody
QQSYSTPGYT

SEQ ID NO: 205 - protein, 12 aa, Artificial sequence
  Synthetic antibody
GDSVSSNSAAWN

SEQ ID NO: 206 - protein, 18 aa, Artificial sequence
  Synthetic antibody
RTYYRSKWYNDYAVSVKS

SEQ ID NO: 207 - protein, 16 aa, Artificial sequence
  Synthetic antibody
EEQQLVHDYYYYGMDV

SEQ ID NO: 208 - protein, 14 aa, Artificial sequence
  Synthetic antibody
TGTSSDIGAYNYIS

SEQ ID NO: 209 - protein, 7 aa, Artificial sequence
  Synthetic antibody
EVSNRPS

SEQ ID NO: 210 - protein, 8 aa, Artificial sequence
  Synthetic antibody
SSYAGSIS

SEQ ID NO: 211 - protein, 10 aa, Artificial sequence
  Synthetic antibody
GGSFSGYYWT

SEQ ID NO: 212 - protein, 16 aa, Artificial sequence
  Synthetic antibody
EINHGGSTRYNPSLES

SEQ ID NO: 213 - protein, 16 aa, Artificial sequence
  Synthetic antibody
GYDTNWYGDGYNWFDP

SEQ ID NO: 214 - protein, 13 aa, Artificial sequence
  Synthetic antibody
SGSSSSIGSNTVH

SEQ ID NO: 215 - protein, 7 aa, Artificial sequence
  Synthetic antibody
EVSNRPS

SEQ ID NO: 216 - protein, 11 aa, Artificial sequence
  Synthetic antibody
QSYDTGLSGHV

SEQ ID NO: 217 - protein, 10 aa, Artificial sequence
  Synthetic antibody
GYTFTGYYMH

SEQ ID NO: 218 - protein, 17 aa, Artificial sequence
  Synthetic antibody
RINPNSGGTNYAQKFQG

SEQ ID NO: 219 - protein, 5 aa, Artificial sequence
  Synthetic antibody
GYFDY

SEQ ID NO: 220 - protein, 13 aa, Artificial sequence
  Synthetic antibody
SGSSSNIGSNSVN

SEQ ID NO: 221 - protein, 7 aa, Artificial sequence
  Synthetic antibody
ANDHRPS

SEQ ID NO: 222 - protein, 11 aa, Artificial sequence
  Synthetic antibody
AAWDNSLKGVV

SEQ ID NO: 223 - protein, 127 aa, Artificial sequence
  Synthetic antibody
QVLLVQSGAEMKKPGASVKVSCKTSGYTFTNSYIHWVRQAPGQGLEWMGWINPISGGTNYAQKFQGRVTMTRDTSINTAYMELSRLRSDDTAVYYCARDRGDYDYGWGTSPFYFDYWGQGTLVTVSS

SEQ ID NO: 224 - protein, 107 aa, Artificial sequence
  Synthetic antibody
DVQMTQSPSSLSASVGDRVTITCRASQSISRYLNWYQQKPGKAPKLLIYAASSLQSGVPSRFSGSGSGADFTLTISSLQPEDFATYYCQQSFSPPITFGQGTRLEIK

SEQ ID NO: 225 - protein, 127 aa, Artificial sequence
  Synthetic antibody
QVQMVQSGAEMKKPGASVKVSCKTSGYTFTNSYIHWVRQAPGQGLEWMGWINPISGGTNYAQKFQGRVTMTRDTSINTAYMELSRLRSDDTAVYYCARDRGDYDYGWGTSPFYFDYWGQGTLVSVSS

SEQ ID NO: 226 - protein, 107 aa, Artificial sequence
  Synthetic antibody
AIQMTQSPSSLSASVGDRVTITCRASQPISKYLNWYQQKPGKAPKLLIYAASSLQSGVPSSFSGSGFGTDFTLTISNLQPEDFATYYCQQSNGIPLTFGGGTKVDIK

SEQ ID NO: 227 - protein, 127 aa, Artificial sequence
  Synthetic antibody
QVQLVESGAEMKKPGASVKVSCKTSGYTFTNSYIHWVRQAPGQGLEWMGWINPISGGTNYAQKFQGRVTMTRDTSINTAYMELSRLRSDDTAVYYCARDRGDYDYGWGTSPFYFDYWGQGTLVTVSS

SEQ ID NO: 228 - protein, 107 aa, Artificial sequence
  Synthetic antibody
DIVMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVDIK

SEQ ID NO: 229 - protein, 124 aa, Artificial sequence
  Synthetic antibody
QVELHQWGAGLLKPSETLSRTCAVYGGSFSGYYWTWIRQAPGKGLEWIGEINHGGSTRYNPSLESRVSISVDTSKKQFSLELRSVTAADTAIYYCARGYDTNWYGDGYNWFDPWDRGTLVTVSS

SEQ ID NO: 230 - protein, 107 aa, Artificial sequence
  Synthetic antibody
NIQMTQSPSSLSASVGDRVTITCRASQSISRYLNWYQQKPGKAPNLLIYAASSLQSGVPSRFSGSGSGTDFTLTISSLQPEDFATYYCQQSFGTPLTFGGGTKVEIK

SEQ ID NO: 231 - protein, 121 aa, Artificial sequence
  Synthetic antibody
EVQLVESGGGVARPGGSLRLSCAASGFTFDDYGMSWVRQVPGKGLEWVAGINWNGERIGYADSVKGRFTISRDNAKNSLYLQMNSLRAEDTALYYCARPSGDYVAWYFNLWGRGTLVTVSS

SEQ ID NO: 232 - protein, 110 aa, Artificial sequence
  Synthetic antibody
QAALTQPRSVSGSPGQSVTISCTGTGSDVGPYKYVSWYQQHPGKAPKLMIYDVNERPSGVPDRFSGSKSGNTASLTISGLQAEDEADYYCGSYAGNNKWVFGGGTKLTVL

SEQ ID NO: 233 - protein, 121 aa, Artificial sequence
  Synthetic antibody
QVELQESGPGLVKPSGTLSLTCAVSGGSISSNNWWTWVRQPPGKGLEWIGEIHHSGGTNYNPSLKSRVTMSVDKSKNQFSLNLYSVTAADTAVYYCTRDRAGGTYSGFDFWGQGTLVTVSS

SEQ ID NO: 234 - protein, 110 aa, Artificial sequence
  Synthetic antibody
QAVVTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVL

SEQ ID NO: 235 - protein, 122 aa, Artificial sequence
  Synthetic antibody
EVQLVESGGGVFQPGRSLRLSCAASGFTFSDFGMHWVRQAPGKGLEWVAVTSHDGSSKFYADSVKGRFTVSRDNSKNKLYLEMNSLRVEDSAVYYCAKDSDNGYDADFFDYWGQGTLVTVSS

SEQ ID NO: 236 - protein, 107 aa, Artificial sequence
  Synthetic antibody
AIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 237 - protein, 124 aa, Artificial sequence
  Synthetic antibody
QVQLHQWGAGLLKPSETLSRTCAVYGGSFSGYYWTWIRQAPGKGLEWIGEINHGGSTRYNPSLESRVSISVDTSKKQFSLELRSVTAADTAIYYCARGYDTNWYGDGYNWFDPWDRGTLVTVSS

SEQ ID NO: 238 - protein, 108 aa, Artificial sequence
  Synthetic antibody
QSALTQDPSVSVALGQTVRITCQGDSLRGSFASWYRQKPGQAPVLVIFGINNRPSGVPDRFSGSSSGNTASLTITGAQAEDEADYYCNSRESNSNRILFGGGTKVTVL

SEQ ID NO: 239 - protein, 122 aa, Artificial sequence
  Synthetic antibody
QVELVESGGGLVQPGRSLRLSCEASGFSFDNYAMHWVRQTPGKGLEWVSGITGNSGTIGYADSVKGRFTISRDNAKNSLYLQMNRLRAEDTALYYCAKDTDYDSSGSYFDYWGQGTLVTVSS

SEQ ID NO: 240 - protein, 108 aa, Artificial sequence
  Synthetic antibody
QPVLTQPPSVAVAPGQTARITCGGKNIGRESVHWYQQKPDQAPVLVIYSDGDRPSGTSERFSGSRSGNAATLTISRVEAGDEADYFCQVWDPDTDHYVFGTGTELTVL

SEQ ID NO: 241 - protein, 123 aa, Artificial sequence
  Synthetic antibody
QVQLVESGGGVVQPGRSLRLSCVASGFTFSSYAMQWVRQAPGKGLEYVAVVSDDGNMKFYADSVKGRFTISRDNSRNTVSLQMNSLGVEDTAVYYCARENYFWSGSIGGLDYWGQGTLVSVSS

SEQ ID NO: 242 - protein, 108 aa, Artificial sequence
  Synthetic antibody
DIQMTQSPSSLSASVGDRVTITCRASQSISSYLNWYQQKPGKAPKLLIYAASSLQSGVPSRFSGSGSGTDFTLTISSLQPEDFATYYCQQSYSTPGYTFGQGTKLEIK

SEQ ID NO: 243 - protein, 119 aa, Artificial sequence
  Synthetic antibody
QVQLQQSGAEVKKPGSSVKVSCKASGGNFRSHTFSWVRQAPGQGLEWMGEIMPRFGATNYAQKFQGRVTITADESTGTTYMELSSLKSHDTAVYFCAADLGSGRKFDSWAQGTLVSVSS

SEQ ID NO: 244 - protein, 107 aa, Artificial sequence
  Synthetic antibody
NIQMTQSPSSLSASVGDRVTITCRASQGIGSDLGWYQQRPGKAPKRLIYAASSLQSGVPSRFSGSGSGTEFTLTISSLQPEDFATYYCLQHNSYPLTFGQGTKVEIK

SEQ ID NO: 245 - protein, 128 aa, Artificial sequence
  Synthetic antibody
EVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREEQQLVHDYYYYGMDVWGQGTMVTVSS

SEQ ID NO: 246 - protein, 108 aa, Artificial sequence
  Synthetic antibody
QAVVTQPASVSGSPGQSITISCTGTSSDIGAYNYISWYQQHPGKAPKLIIYEVSNRPSGISYRFSGSKSGNTASLTISGLQAEDEANYYCSSYAGSISFGGGTKVTVL

SEQ ID NO: 247 - protein, 128 aa, Artificial sequence
  Synthetic antibody
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREEQQLVHDYYYYGMDVWGQGTTVTVSS

SEQ ID NO: 248 - protein, 104 aa, Artificial sequence
  Synthetic antibody
QSALTQPPSASGSPGQSVTISCTGISSDYVSWYQQHPGKAPKLMIYEVNKRPSGVPDRFSGSKSGNTASLTVSGLQAEDEADYYCGSYAGSNTFFGGGTKLTVL

SEQ ID NO: 249 - protein, 124 aa, Artificial sequence; identical to SEQ ID NO: 237
  Synthetic antibody

SEQ ID NO: 250 - protein, 110 aa, Artificial sequence
  Synthetic antibody
QAVVTQPPSVSGTPGQRVTISCSGSSSSIGSNTVHWYQHLPGTAPKLLMYNNNERPSGVPARFSGSKSGTSASLVITGLQADDEAEYYCQSYDTGLSGHVFGSGTELTVL

SEQ ID NO: 251 - protein, 125 aa, Artificial sequence
  Synthetic antibody
QVELVESGGGLVKPGGSLRLSCAASGFTFSSYSMNWVRQAPGKGLEWVSSISSSSSYIYYADSVKGRFTISRDNAKNSLYLQMNSLRAEDTAVYYCARGNVDIVATGEVDAFDIWGQGTMVSVSS

SEQ ID NO: 252 - protein, 110 aa, Artificial sequence
  Synthetic antibody
QAVVTQPPSASGTLGQRVTISCSGSSSTIGSNYVYWYQQLPGTAPKLLIYRNNQRPSGVPDRFSGSKSGTSASLAISGLRSEDEADYYCAAWDDSLSGYVFGTGTQLTVL

SEQ ID NO: 253 - protein, 126 aa, Artificial sequence
  Synthetic antibody
QVQLVQSGAEVKKPGSSVKVSCKASGGTFSNYGISWVRQAPGQGLEWMGGIIPIFGTANYAQKFQGRVTITADKSTRTAYMELSSLRFEDTAVYYCARDRDDALTGLGLGGGFDIWGQGTMVTVSS

SEQ ID NO: 254 - protein, 110 aa, Artificial sequence
  Synthetic antibody
QAVVTQPPSASGTPGQRVTISCSGSTSNIGTNTANWYQHLPGTAPKLLIYGNDRRPSGVPDRFSGSKSVTSASLAISGLQSEDEADYYCAAWDERLNGYVFGTGTKLTVL

SEQ ID NO: 255 - protein, 127 aa, Artificial sequence
  Synthetic antibody
QVQLVQSGAEMKKPGASVKVSCKTSGYTFTNSYIHWVRQAPGQGLEWMGWINPISGGTNYAQKFQGRVTMTRDTSINTAYMELSRLRSDDTAVYYCARDRGDYDYGWGTSPFYFDYWGQGTLVTVSS

SEQ ID NO: 256 - protein, 107 aa, Artificial sequence
  Synthetic antibody
AIQLTQSPSSLSASVGDRVTITCRTSHTVNSYLNWYQQEPGKAPKLLIYAASNLQSGVPSRFSGSGSGTDFSLTIHNLQPEDFATYYCQQSYRTPLTFGQGTKVDIK

SEQ ID NO: 257 - protein, 121 aa, Artificial sequence
  Synthetic antibody
QVQLVESGGDLVQPGRSLRLSCAASGFIFDDYAMHWVRQVPGKGPEWVSGVNRDSTYVGQADSVKGRFTISRDNAKKALFLQMDSLRVEDTALYYCVRGMTRVATDAFDFWGQGTMVTVSS

SEQ ID NO: 258 - protein, 108 aa, Artificial sequence
  Synthetic antibody
AIQMTQSPSSLSASVGDRVTITCRASQSINGYLNWYQQKPGKAPKLLIYSASRLQSGVPSRFSGSGSGADYTLTISNLQVEDFATYYCQQSYSTPPVTFGQGTRLEIK

SEQ ID NO: 259 - protein, 127 aa, Artificial sequence; identical to SEQ ID NO: 255
  Synthetic antibody

SEQ ID NO: 260 - protein, 107 aa, Artificial sequence
  Synthetic antibody
DVQMTQSPSSLSASVGDRVTITCRASQSISSYLNWYQQKPGKAPKLLIYGASSLESGVPSRFSGGGSGTDFTLTISSLQPEDFASYYCQQSYSIPFTFGPGTTVDIK

SEQ ID NO: 261 - protein, 121 aa, Artificial sequence
  Synthetic antibody
QVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDMDIWFGGGLDYWGQGTLVSVSS

SEQ ID NO: 262 - protein, 107 aa, Artificial sequence
  Synthetic antibody
DVQMTQSPSSVSASVGDRVTITCRASEGIGNWLAWYQQKPGKAPKLLIYEASSLQSGVPSRFSGSGSGADFTLTISSLQPEDFGTYYCLQANSFPITFGRGTKLEIK

SEQ ID NO: 263 - protein, 118 aa, Artificial sequence
  Synthetic antibody
QVQLHQWGAGLLKPSETLSRTCAVYGGSFSGYYWTWIRQAPGKGLEWIGEINHGGSTRYNPSLESRVSISVDTSKKQFSLELRSVTAADTAIYYCARGLVGGGAFDIWGQGTMVTVSS

SEQ ID NO: 264 - protein, 108 aa, Artificial sequence
  Synthetic antibody
NFMLTQPSSVSVAPGQTARITCGGDNIGSTGVHWYQQKPGQAPVLVVYDDTDRPSGIPERFSGSKSGNTAALTISGVEAGDEADFYCQVWDSSGHSYVFGTGTKVTVL

SEQ ID NO: 265 - protein, 114 aa, Artificial sequence
  Synthetic antibody
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCARGYFDYWGQGTLVTVSS

SEQ ID NO: 266 - protein, 110 aa, Artificial sequence
  Synthetic antibody
QAVVTQPPSTSGTPGQRVTISCSGSSSNIGSNTVNWYQQLPGTAPKLLIYSNNQRPSGVPDRFSGSKSGTSASLAISGLQSEDEADYYCASWDDSLNGVVFGGGTKVTVL

SEQ ID NO: 267 - protein, 114 aa, Artificial sequence; identical to SEQ ID NO: 265
  Synthetic antibody

SEQ ID NO: 268 - protein, 110 aa, Artificial sequence
  Synthetic antibody
QSALTQPPSASGTPGQRVTISCSGSSSNIGSNSVNWYQQVPGTAPKLLIYANDHRPSGVPDRFSGSKSGTSASLAISGLQSEDEADYYCAAWDNSLKGVVFGGGTKVTVL

SEQ ID NO: 269 - protein, 124 aa, Artificial sequence; identical to SEQ ID NO: 229
  Synthetic antibody

SEQ ID NO: 270 - protein, 110 aa, Artificial sequence
  Synthetic antibody
QSVLTQPPSASGTPGQRVTISCSGRSSNIGTNPVNWYQQFPGTAPKLLVYYNNQRPSGVPDRFSGSKSGTSASLAISGLQSEDEADYYCAAWDDSLKGWVFGGGTKVTVL

SEQ ID NO: 271 - protein, 122 aa, Artificial sequence
  Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDPYSGSYPGAFDIWGQGTTVTVSS

SEQ ID NO: 272 - protein, 110 aa, Artificial sequence
  Synthetic antibody
QAVVTQPASVSGSPGQSITISCTGTSSDVGGYNYVSWYQQHPGKAPKLMIYDVSNRPSGVSNRFSGSKSGNTASLTISGLQAEDEADYYCNSYTRSSTSVFGTGTQLTVL

SEQ ID NO: 273 - protein, 126 aa, Artificial sequence
  Synthetic antibody
QVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARMADDDFWRDLPGFYMDVWGKGTTVTVSS

SEQ ID NO: 274 - protein, 110 aa, Artificial sequence
  Synthetic antibody
QAVVTQPASVSGSPGQSITISCTGTSSDVGGYNYVSWYQQHPGKAPKLMIYDVSNRPSGVSNRFSGSKSGNTASLTISGLQAEDEADYYCSSYTSSSTLVFGGGTKVTVL

SEQ ID NO: 275 - protein, 124 aa, Artificial sequence
  Synthetic antibody
QVQLQQWGAGLLKPSETLSRTCAVYGGSFSGYSWTWIRQAPGKGLEWIGEINHGGSTNYNPSLESRVSISVDTSKKQFSLELRSVTAADTAVYYCARGYDTNWYGDGYNWFDPWGQGTLVTVSS

SEQ ID NO: 276 - protein, 106 aa, Artificial sequence
  Synthetic antibody
QAVVTQPLSVSVALGQTAKIPCGGNNIGRKSVHWYQQKPGQAPVLVIYRDNNRPSGIPERFSGSNSGNTATLTISRAQAGDEADYYCQVWDSNTGVFGTGTKVTVL

SEQ ID NO: 277 - protein, 123 aa, Artificial sequence
  Synthetic antibody
EVQLVESGGGLVQPGRSLRLSCAAPRFTFDDYAIHWVRQVPGKGLEWVSGSSWNSGTIGYAPSVKGRFTISRDNAKNSLFLQMNSLRVEDTALYYCAILPGDYNRVADVFDIWGQGTMVIVSS

SEQ ID NO: 278 - protein, 111 aa, Artificial sequence
  Synthetic antibody
QAVVTQPPSVSGAPGQRVTISCTGSSSNIGAGYDVHWYQQLPGTAPKLLIYGNNNRPSGVPDRFSGSKSGTSASLAITGLQVEDEADYYCQSYDSSLSGYVFGTGTKVTVL

SEQ ID NO: 279 - protein, 123 aa, Artificial sequence
  Synthetic antibody
QVQLVESGGGLVQPGRSLRLSCAASRFTFDDHAMHWVRQAPGKGLEWVSGISWNGGIIGYADSVKGRFTISRDNAKNSLYLQMNSLRAEDTALYYCAKGMGFGESNYYAMDVWGQGTMVTVSS

SEQ ID NO: 280 - protein, 111 aa, Artificial sequence
  Synthetic antibody
QSALTQPASVSGSPGQSITISCTGTSSDVGGYNYVSWYQQHPGKAPKLIIYDVSNRPSGVSNRFSGSKSGNTASLTISGLQAEDEAYYYCSSYTSISNLVVFGGGTKVTVL

SEQ ID NO: 281 - protein, 124 aa, Artificial sequence
  Synthetic antibody
QVQLHQWGAGLLKPSETLSRTCAVYGGSFSGYSWTWIRQAPGKGLEWIGEVNHGGKTNYNPSLESRLSISVDTSKNQFSLELRSVTAADTAVYYCARGYDTNWYGDGYNWFDPWGQGTLVSVSS

SEQ ID NO: 282 - protein, 107 aa, Artificial sequence
  Synthetic antibody
DIQMTQSPSSLSASVGDRVTITCRASQKIDNFLNWYQQKPGKAPKLLIHGASNLQGGVSSRFSGSRSGTDFTLTITNLQPEDFATYYCQQSYNTPITFGQGTRLEIK

SEQ ID NO: 283 - protein, 124 aa, Artificial sequence
  Synthetic antibody
QVQLHQWGAGLLKPSETLSRTCAVYGGSFSGYSWTWIRQAPGKGLEWIGEINHGGSTKYNPSLESRVTISVDTSKKQFSLELRSVTAADTAVYYCARGYDTNWYGDGYNWFDPWGQGTLVSVSS

SEQ ID NO: 284 - protein, 107 aa, Artificial sequence
  Synthetic antibody
NFMLTQPASVSVAPGKTASITCGGDNIGTKSVHWYRQKPGQAPVLVIYYDSDRPSGIPERFSGSNSGNTATLTISRVEAGDEADYYCQVGDSSSHVVFGGGTKVTVL

SEQ ID NO: 285 - DNA, 381 bp, Artificial sequence
  Synthetic antibody
caggtgcttctggtgcagtctggggctgagatgaagaagcctggggcctcagtgaaggtctcctgcaagacttctggatacaccttcaccaactcctatatacactgggtgcgacaggcccctggacaagggcttgagtggatgggatggatcaaccctatcagtggtggcacaaactatgcacagaagtttcagggcagggtcaccatgaccagggacacgtccatcaacacagcctacatggagctgagcaggctgagatctgacgacacggccgtgtattactgtgcgagagatcggggggattatgattacggttgggggacttctcctttctactttgactactggggccagggcaccctggtcaccgtctcctca

SEQ ID NO: 286 - DNA, 321 bp, Artificial sequence
  Synthetic antibody
gacgtccagatgacccagtctccatcctccctgtctgcatctgtgggagacagagtcaccatcacttgccgggcaagtcagagcattagtaggtatttaaattggtatcaacagaaaccagggaaagcccctaagctcctgatctatgctgcatccagtttgcaaagtggggtcccatcaaggttcagtggcagtggatctggggcagatttcactctcaccatcagcagtctgcaacctgaagattttgcaacttattactgtcaacagagtttcagtcccccgatcaccttcggccaagggacacgactggagatcaaa

SEQ ID NO: 287 - DNA, 381 bp, Artificial sequence
  Synthetic antibody
caggtgcagatggtgcagtctggggctgagatgaagaagcctggggcctcagtgaaggtctcctgcaagacttctggatacaccttcaccaactcctatatacactgggtgcgacaggcccctggacaagggcttgagtggatgggatggatcaaccctatcagtggtggcacaaactatgcacagaagtttcagggcagggtcaccatgaccagggacacgtccatcaacacagcctacatggagctgagcaggctgagatctgacgacacggccgtgtattactgtgcgagagatcggggggattatgattacggttgggggacttctcctttctactttgactactggggccagggaaccctggtcagcgtctcctca

SEQ ID NO: 288 - DNA, 321 bp, Artificial sequence
  Synthetic antibody
gccatccagatgacccagtctccatcctccctgtctgcgtctgtaggggacagagtcaccatcacctgccgggcaagtcagcccattagtaagtatttaaattggtatcagcagaaaccagggaaagcccctaagctcctgatctatgctgcatccagtttgcaaagtggggtcccatcaagtttcagtggcagtggatttgggacagatttcactctcaccatcagcaatctgcaacctgaagattttgcaacttactactgtcaacagagcaacggtatcccgctcactttcggcggagggaccaaagtggatatcaaa

SEQ ID NO: 289 - DNA, 381 bp, Artificial sequence
  Synthetic antibody
caggtgcagctggtggagtctggggctgagatgaagaagcctggggcctcagtgaaggtctcctgcaagacttctggatacaccttcaccaactcctatatacactgggtgcgacaggcccctggacaagggcttgagtggatgggatggatcaaccctatcagtggtggcacaaactatgcacagaagtttcagggcagggtcaccatgaccagggacacgtccatcaacacagcctacatggagctgagcaggctgagatctgacgacacggccgtgtattactgtgcgagagatcggggggattatgattacggttgggggacttctcctttctactttgactactggggccagggaaccctggtcaccgtctcttca

SEQ ID NO: 290 - DNA, 321 bp, Artificial sequence
  Synthetic antibody
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccctgacgttcggccaagggaccaaagtggatatcaaa

SEQ ID NO: 291 - DNA, 372 bp, Artificial sequence
  Synthetic antibody
caggtggagctacatcagtggggcgcaggactgttgaagccttcggagaccctgtcccgcacctgcgctgtctatggtgggtccttcagtgggtactactggacctggatccgccaggccccggggaaggggctggaatggattggggaaatcaatcatggtggaagtaccagatataatccgtccctcgagagtcgagtcagcatatcagtcgacacgtccaagaaacagttctccctggaactgaggtctgtgaccgccgcggacacggctatttattactgtgcgagaggctatgacaccaactggtatggtgacggatacaattggttcgacccctgggaccggggaaccctggtcaccgtctcctca

SEQ ID NO: 292 - DNA, 321 bp, Artificial sequence
  Synthetic antibody
aacatccagatgacccagtctccatcctccctgtctgcatctgtaggagacagagtcaccatcacttgccgggcaagtcagagcatttccaggtatttaaattggtatcagcagaaaccagggaaagcccctaacctcctgatctatgctgcatccagtttgcaaagtggggtcccatcaaggttcagtggcagtggatctgggacagatttcactctcaccatcagcagtctgcaacctgaagattttgcaacttactactgtcaacaaagttttggtaccccgctcactttcggcggagggaccaaggtggaaatcaaa

SEQ ID NO: 293 - DNA, 363 bp, Artificial sequence
  Synthetic antibody
gaagtgcagctggtggagtcggggggaggtgtggcacggcctggggggtccctgagactctcctgtgcagcctccggattcacctttgatgattatggaatgagctgggtccgccaggttccaggaaaggggctggagtgggtcgctggtattaattggaatggtgagagaataggttatgcagactctgtgaagggccgattcaccatctccagagacaacgccaagaactccctgtatctgcagatgaacagtctgagagccgaggacacggccttatattattgtgcgaggccctccggtgactacgttgcctggtacttcaatctctggggccgcggcaccctggtcaccgtctcctca

SEQ ID NO: 294 - DNA, 330 bp, Artificial sequence
  Synthetic antibody
caggctgccctgactcagcctcgctcagtgtccgggtctcctggacagtcagtcaccatctcctgcactggaaccggcagtgatgttggtccttataagtatgtctcctggtaccaacaacacccaggcaaagcccccaaactcatgatttatgatgtcaatgagcggccctcaggggtccctgatcgcttctctggctccaagtctggcaacacggcctccctgaccatctctgggctccaggctgaggatgaagctgattattactgcggctcatatgcaggcaacaacaagtgggtgttcggcggagggaccaagctgaccgtccta

SEQ ID NO: 295 - DNA, 363 bp, Artificial sequence
  Synthetic antibody
caggtggagctgcaggagtcgggcccaggtctggtgaagccttcggggaccctgtcgctcacctgcgctgtctctggtggctccatcagcagtaataactggtggacttgggtccgccagcccccagggaaggggctggagtggattggcgaaatccatcatagtgggggcaccaactacaacccgtccctcaagagtcgagtcaccatgtcagttgacaagtctaagaaccagttctccctgaacctgtactctgtgaccgccgcggacacggccgtctattattgtacgagagaccgagcaggtgggacgtatagtggctttgacttctggggccagggaaccctggtcaccgtctcttca

SEQ ID NO: 296 - DNA, 330 bp, Artificial sequence
  Synthetic antibody
caggctgtggtgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcaccgtggtattcggcggagggaccaaggtcaccgtccta

SEQ ID NO: 297 - DNA, 366 bp, Artificial sequence
  Synthetic antibody
gaagtgcagctggtggagtctgggggaggcgtgttccagcctgggaggtccctgagactctcctgtgcagcctctggattcaccttcagtgactttggcatgcattgggtccgccaggctccaggcaaggggctggagtgggtggcagttacatcacatgatggaagtagtaagttctatgcagactccgtgaagggccgattcaccgtctccagagacaattccaagaacaagctgtatctggaaatgaacagcctgagagttgaggactcggctgtttattactgtgcgaaagattccgataatggctacgatgcagacttctttgactactggggccagggaaccttggtcaccgtctcctca

SEQ ID NO: 298 - DNA, 321 bp, Artificial sequence
  Synthetic antibody
gccatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgctcactttcggcggagggaccaagctggagatcaaa

SEQ ID NO: 299 - DNA, 372 bp, Artificial sequence
  Synthetic antibody
caggtgcagctgcatcagtggggcgcaggactgttgaagccttcggagaccctgtcccgcacctgcgctgtctatggtgggtccttcagtgggtactactggacctggatccgccaggccccggggaaggggctggaatggattggggaaatcaatcatggtggaagtaccagatataatccgtccctcgagagtcgagtcagcatatcagtcgacacgtccaagaaacagttctccctggaactgaggtctgtgaccgccgcggacacggctatttattactgtgcgagaggctatgacaccaactggtatggtgacggatacaattggttcgacccctgggaccggggaaccctggtcaccgtctcctca

SEQ ID NO: 300 - DNA, 324 bp, Artificial sequence
  Synthetic antibody
cagtctgccctgactcaggacccttctgtgtctgtggccttgggacagacagtcaggatcacatgccaaggagacagtttaagaggctcttttgcaagttggtaccggcagaagccaggacaggcccctgtccttgtcatctttgggataaataaccggccctcaggggtccccgaccgattctctggctccagttcaggaaacacagcttccttgaccatcactggggctcaggcggaagatgaggctgactactactgtaattcccgggagagcaattctaatcgtatactattcggcggagggaccaaggtcaccgtccta

SEQ ID NO: 301 - DNA, 366 bp, Artificial sequence
  Synthetic antibody
caggtggagctggtggagtctgggggaggcttggtacagcctggcaggtccctgagactctcctgtgaagcctctggattcagctttgataattatgccatgcactgggtccggcaaactccagggaagggcctggagtgggtctcaggtattactgggaatagtggtaccataggctatgcggactctgtgaagggccggttcaccatctccagagacaacgccaagaactccctgtatctgcaaatgaaccgtctgagagctgaggacacggccttgtattattgtgcaaaagataccgactatgatagtagcgggagttactttgactactggggccagggaaccctggtcaccgtctcctca

SEQ ID NO: 302 - DNA, 324 bp, Artificial sequence
  Synthetic antibody
cagcctgtgctgactcagccaccctcagtggcagtggccccggggcagacggccaggattacctgtgggggcaaaaatattggacgtgaaagtgtgcactggtaccagcagaagccagaccaggcccctgtcctggtcatctactctgatggcgaccggccctcagggacctctgagcgattctctggctccaggtctgggaacgcggccaccctgacaatcagtagggtcgaggccggggatgaggccgactatttctgtcaggtgtgggatcctgatactgatcactatgtgttcggtactgggaccgagctgaccgtccta

SEQ ID NO: 303 - DNA, 369 bp, Artificial sequence
  Synthetic antibody
caggtgcagctggtggagtctgggggaggcgtggtccagcctgggaggtccctgagactctcctgtgtagcctccggattcacgtttagtagttatgccatgcagtgggtccgccaggccccaggcaaggggctggagtacgtggcagttgtttcagatgatggaaacatgaaattctacgcagactccgtgaagggccgattcaccatctccagagacaattccaggaacacggtgtctctgcaaatgaacagcctgggagtcgaggacacggccgtatattactgtgcgagagaaaattacttttggtcggggagtataggcgggcttgactactggggccagggaaccctggtcagcgtctcctca

SEQ ID NO: 304 - DNA, 324 bp, Artificial sequence
  Synthetic antibody
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccggggtacacttttggccaggggaccaagctggagatcaaa

SEQ ID NO: 305 - DNA, 357 bp, Artificial sequence
  Synthetic antibody
caggtgcagctgcagcagtctggggctgaggtgaagaagcctgggtcctcggtgaaggtctcctgcaaggcttctggaggcaacttcaggagtcatactttcagctgggtgcgacaggcccctggacaagggcttgagtggatgggagagatcatgcctcgctttggtgcaacaaactacgcacagaagttccagggcagagtcaccattaccgcggacgaatctacaggcacgacctacatggagctgagcagccttaagtctcatgacacggccgtgtacttctgtgcggcagatctcggttcggggaggaaatttgactcctgggcccagggaaccctggtcagcgtctcctca

SEQ ID NO: 306 - DNA, 321 bp, Artificial sequence
  Synthetic antibody
aacatccagatgacccagtctccatcctccctgtctgcatctgtaggagacagagtcaccatcacttgccgggcaagtcagggcattggaagtgatttaggctggtatcagcagagaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacaacataatagttaccctctgacgttcggccaagggaccaaagtggaaatcaaa

SEQ ID NO: 307 - DNA, 384 bp, Artificial sequence
  Synthetic antibody
gaagtgcagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagaggagcagcagctggtacatgactactactactacggtatggacgtctggggccaagggacaatggtcaccgtctcttca

SEQ ID NO: 308 - DNA, 324 bp, Artificial sequence
  Synthetic antibody
caggctgtggtgactcagcctgcctccgtgtctgggtctcctggacagtcgatcaccatctcctgcactggaaccagcagtgacattggtgcttataactatatctcctggtatcaacagcacccaggcaaagcccccaaactcataatttatgaggtcagtaatcggccctcagggatttcttatcgcttctctggctccaagtctggcaacacggcctccctgaccatctctgggctccaggctgaggacgaggctaattattactgcagctcatatgcaggcagcatctcattcggcggagggaccaaggtcaccgtccta

SEQ ID NO: 309 - DNA, 384 bp, Artificial sequence
  Synthetic antibody
caggtgcagctacagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagaggagcagcagctggtacatgactactactactacggtatggacgtctggggccaagggaccacggtcaccgtctcctca

SEQ ID NO: 310 - DNA, 312 bp, Artificial sequence
  Synthetic antibody
cagtctgccctgactcagcctccctccgcgtccgggtctcctggacagtcagtcaccatctcctgcactggaatcagcagtgactatgtctcctggtaccaacagcacccaggcaaagcccccaaactcatgatttatgaggtcaataagcggccctcaggggtccctgatcgcttctctgggtccaagtctggcaacacggcctccctgaccgtctctgggctccaggctgaagatgaggctgattattactgcggctcatatgcaggcagcaacacgttcttcggcggagggaccaagctgaccgtccta

SEQ ID NO: 311 - DNA, 372 bp, Artificial sequence
  Synthetic antibody
caggtgcagctgcatcagtggggcgcaggactgttgaagccttcggagaccctgtcccgcacctgcgctgtctatggtgggtccttcagtgggtactactggacctggatccgccaggccccggggaaggggctggaatggattggggaaatcaatcatggtggaagtaccagatataatccgtccctcgagagtcgagtcagcatatcagtcgacacgtccaagaaacagttctccctggaactgaggtctgtgaccgccgcggacacggctatttattactgtgcgagaggctatgacaccaactggtatggtgacggatacaattggttcgacccctgggaccggggaaccctggtcaccgtctcttca

SEQ ID NO: 312 - DNA, 330 bp, Artificial sequence
  Synthetic antibody
caggctgtggtgacgcagccaccctcagtgtctgggacccccggacagagggtcaccatctcttgttctggaagcagttccagcatcggaagtaatactgtacactggtatcagcacctcccaggaacggcccccaaactcctcatgtataataataatgagcggccctcaggggtccctgcccggttctctggctccaagtctggcacctcagcctccctggtcatcactgggctccaggctgacgatgaggctgaatattactgccagtcctatgacaccggcctgagcggtcatgtcttcggaagtgggaccgagctgaccgtccta

SEQ ID NO: 313 - DNA, 375 bp, Artificial sequence
  Synthetic antibody
caggtggagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaggcaacgtggatatagtggctacgggcgaagttgatgcttttgatatctggggccaagggacaatggtcagcgtctcctca

SEQ ID NO: 314 - DNA, 330 bp, Artificial sequence
  Synthetic antibody
caggctgtggtgactcagccaccctcagcgtctgggaccctcgggcagagggtcaccatctcttgttctggaagcagctccaccatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttatgtcttcggaactgggacccagctgaccgtccta

SEQ ID NO: 315 - DNA, 378 bp, Artificial sequence
  Synthetic antibody
caggtgcagctggtgcagtctggggctgaggtgaagaagcctgggtcctcggtgaaggtctcctgcaaggcttctggaggcaccttcagcaattatggtatcagctgggtgcgacaggcccctggacaggggcttgagtggatgggaggaatcatccctatctttggtacagcaaactacgcacagaagttccagggcagagtcacgattaccgcggacaaatccacgagaacagcctatatggaactgagcagcctgagatttgaggacacggccgtttattactgtgcgagagatcgggacgatgctttgactggcctgggcctagggggtggttttgatatctggggccaagggacaatggtcaccgtctcctca

SEQ ID NO: 316 - DNA, 330 bp, Artificial sequence
  Synthetic antibody
caggctgtggtgactcagccaccctcagcgtctgggacccccgggcagagggtcaccatctcttgttctggaagcacctccaatatcggaactaatactgcaaactggtaccagcacctcccaggaacggcccccaaactcctcatctatggtaatgatcggcgaccctcaggggtccctgaccgattctctggctccaagtctgtcacctcagcctccctggccatcagtgggctccagtctgaggatgaggctgattattactgtgcagcatgggatgagagactgaatgggtatgtcttcggaactgggaccaagctgaccgtccta

SEQ ID NO: 317 - DNA, 381 bp, Artificial sequence
  Synthetic antibody
caggtgcagctggtgcagtctggggctgagatgaagaagcctggggcctcagtgaaggtctcctgcaagacttctggatacaccttcaccaactcctatatacactgggtgcgacaggcccctggacaagggcttgagtggatgggatggatcaaccctatcagtggtggcacaaactatgcacagaagtttcagggcagggtcaccatgaccagggacacgtccatcaacacagcctacatggagctgagcaggctgagatctgacgacacggccgtgtattactgtgcgagagatcggggggattatgattacggttgggggacttctcctttctactttgactactggggccagggaaccctggtcaccgtctcctca

SEQ ID NO: 318 - DNA, 321 bp, Artificial sequence
  Synthetic antibody
gccatccagttgacccagtctccatcctccctgtctgcatctgtaggcgacagagtcaccatcacttgccggacaagtcacaccgttaacagctatctaaattggtatcaacaagaaccagggaaagcccctaaactcctgatctatgctgcatccaatttgcaaagtggggtcccgtcaaggttcagtggcagtggatctgggacagatttctctctcaccatccacaatctgcaacctgaagattttgcaacttactactgtcaacagagttacagaacccctctcacttttggccaggggaccaaagtggatatcaaa

SEQ ID NO: 319 - DNA, 363 bp, Artificial sequence
  Synthetic antibody
caggtgcagctggtggagtctgggggggacttagtgcagcctggcaggtccctgagactctcctgtgcagcctctggattcatctttgacgattatgccatgcactgggtccggcaagttccagggaagggcccggagtgggtctcaggtgttaatcgggacagtacttatgtcggccaggcagactctgtgaagggccgattcaccatctccagagacaacgccaagaaggccctgtttctgcaaatggacagtttgagagttgaggacacggccttatattattgtgtcaggggaatgacaagggtggcaactgacgcctttgatttctggggccaggggacaatggtcaccgtctcctca

SEQ ID NO: 320 - DNA, 324 bp, Artificial sequence
  Synthetic antibody
gccatccagatgacccagtctccatcctccctgtctgcgtctgtaggagacagagtcaccatcacttgccgggcaagtcagagcattaatggctatttaaactggtatcagcagaaaccagggaaagcccctaagctcctgatctattctgcatctcgtttgcaaagtggggtcccatcaaggttcagtggcagtggatctggggcagattacactctcaccatcagcaatctgcaagttgaagattttgcaacttactactgtcaacagagctacagtacccctccggtcaccttcggccaagggacacgactggagattaaa

SEQ ID NO: 321 - DNA, 381 bp, Artificial sequence
  Synthetic antibody
caggtgcagctggtgcagtctggggctgagatgaagaagcctggggcctcagtgaaggtctcctgcaagacttctggatacaccttcaccaactcctatatacactgggtgcgacaggcccctggacaagggcttgagtggatgggatggatcaaccctatcagtggtggcacaaactatgcacagaagtttcagggcagggtcaccatgaccagggacacgtccatcaacacagcctacatggagctgagcaggctgagatctgacgacacggccgtgtattactgtgcgagagatcggggggattatgattacggttgggggacttctcctttctactttgactactggggccagggaaccctggtcaccgtctcttca

SEQ ID NO: 322 - DNA, 321 bp, Artificial sequence
  Synthetic antibody
gacgtccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttggaaagtggggtcccatcaaggttcagtggcggtggatctgggacagatttcactctcaccatcagcagtctgcaacctgaagattttgcaagttactactgtcaacagagttacagtatccctttcactttcggccctgggactacagtggatatcaaa

SEQ ID NO: 323 - DNA, 363 bp, Artificial sequence
  Synthetic antibody
caggtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatatggatatatggttcgggggcggtctagactactggggccagggaaccctggtcagcgtctcctca

SEQ ID NO: 324 - DNA, 321 bp, Artificial sequence
  Synthetic antibody
gacgtccagatgacccagtctccatcttccgtgtctgcatctgtaggagacagagtcaccatcacttgtcgggcgagtgagggtatagggaattggttagcctggtatcagcagaaaccagggaaagcccctaaactcctgatctatgaagcatccagtttgcaaagtggggtcccatcgaggttcagcggcagtggatctggggctgacttcactctcaccatcagcagcctgcagcctgaagattttggaacttactattgtctacaggctaacagtttcccgatcaccttcggccgggggaccaagctggagatcaaa

SEQ ID NO: 325 - DNA, 354 bp, Artificial sequence
  Synthetic antibody
caggtgcagctgcatcagtggggcgcaggactgttgaagccttcggagaccctgtcccgcacctgcgctgtctatggtgggtccttcagtgggtactactggacctggatccgccaggccccggggaaggggctggaatggattggggaaatcaatcatggtggaagtaccagatataatccgtccctcgagagtcgagtcagcatatcagtcgacacgtccaagaaacagttctccctggaactgaggtctgtgaccgccgcggacacggctatttattactgtgcgagaggcctcgtgggaggcggagcttttgatatctggggccaagggacaatggtcaccgtctcctca

SEQ ID NO: 326 - DNA, 324 bp, Artificial sequence
  Synthetic antibody
aattttatgctgactcagccatcctcggtgtcagtggccccaggacagacggccaggattacctgtgggggagacaacattggaagtacaggtgtgcactggtaccagcagaagccaggccaggcccctgtgttggtcgtctatgatgatactgaccggccctcagggatccctgagcgattctctggctccaaatctgggaacacggccgccctgaccattagcggggtcgaagccggggatgaggccgacttttattgtcaggtgtgggatagtagtggtcattcttatgtcttcggaactgggaccaaggtcaccgtccta

SEQ ID NO: 327 - DNA, 342 bp, Artificial sequence
  Synthetic antibody
caggtgcagctggtggagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaggatactttgactactggggccagggcaccctggtcaccgtctcttca

SEQ ID NO: 328 - DNA, 330 bp, Artificial sequence
  Synthetic antibody
caggctgtggtgactcagccaccctcaacgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattattgtgcatcatgggatgacagcctgaatggtgtggtattcggcggagggaccaaggtcaccgtccta

SEQ ID NO: 329 - DNA, 342 bp, Artificial sequence; identical to SEQ ID NO: 327
  Synthetic antibody

SEQ ID NO: 330 - DNA, 330 bp, Artificial sequence
  Synthetic antibody
cagtctgccctgactcagccaccctcagcgtctgggacccccgggcagagggtcaccatctcttgttctggaagcagctccaacatcggaagtaattctgtaaactggtaccagcaagtcccaggaacggcccccaaactcctcatctatgctaatgatcaccggccctcaggggtccctgaccgattctctggctccaagtctggcacctcagcctccctggccatcagtgggctccagtctgaggatgaggctgattattactgtgcagcatgggataacagcctgaaaggtgtggtgttcggcggagggaccaaggtcaccgtccta

SEQ ID NO: 331 - DNA, 372 bp, Artificial sequence
  Synthetic antibody
caggtggagctacatcagtggggcgcaggactgttgaagccttcggagaccctgtcccgcacctgcgctgtctatggtgggtccttcagtgggtactactggacctggatccgccaggccccggggaaggggctggaatggattggggaaatcaatcatggtggaagtaccagatataatccgtccctcgagagtcgagtcagcatatcagtcgacacgtccaagaaacagttctccctggaactgaggtctgtgaccgccgcggacacggctatttattactgtgcgagaggctatgacaccaactggtatggtgacggatacaattggttcgacccctgggaccggggaaccctggtcaccgtctcttca

SEQ ID NO: 332 - DNA, 330 bp, Artificial sequence
  Synthetic antibody
cagtctgtgctgacacagccaccctcagcgtctgggacccccgggcagagggtcaccatctcttgttctggaaggagctccaacatcggaactaatcctgtaaactggtaccagcagttcccaggaacggcccccaaactcctcgtctattataataatcagcggccctcaggggtccctgaccgattctctggctccaagtctggcacctcagcctccctggccatcagtgggctccagtctgaggatgaggctgattattattgtgcagcgtgggatgacagcctgaagggttgggtgttcggcggagggaccaaggtcaccgtccta

SEQ ID NO: 333 - DNA, 366 bp, Artificial sequence
  Synthetic antibody
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagacccctatagtgggagctaccccggggcttttgatatctggggccaagggaccacggtcaccgtctcttca

SEQ ID NO: 334 - DNA, 330 bp, Artificial sequence
  Synthetic antibody
caggctgtggtgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcaactcatatacaaggagcagcacctctgtcttcggaactgggacccagctgaccgtccta

SEQ ID NO: 335 - DNA, 378 bp, Artificial sequence
  Synthetic antibody
caggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggccgtgtattactgtgcgagaatggcagatgacgatttttggagggacctacccgggttctacatggacgtctggggcaaagggaccacggtcaccgtctcctca

SEQ ID NO: 336 - DNA, 330 bp, Artificial sequence
  Synthetic antibody
caggctgtggtgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactctggtattcggcggagggaccaaggtcaccgtccta

SEQ ID NO: 337 - DNA, 372 bp, Artificial sequence
  Synthetic antibody
caggtgcagctacagcagtggggcgcaggactgttgaagccttcggagaccctgtcccgcacctgcgctgtctatggtgggtccttcagtggttattcctggacctggatccgccaggccccggggaaggggctggaatggattggggaaatcaatcatggtggaagcaccaactacaacccgtccctcgagagtcgagtcagtatatcagtagacacgtccaagaaacagttctccctggaactgaggtctgtgaccgccgcggacacggctgtttattattgtgcgagaggatatgataccaactggtatggtgacggctacaattggttcgacccctggggccagggcaccctggtcaccgtctcttca

SEQ ID NO: 338 - DNA, 318 bp, Artificial sequence
  Synthetic antibody
caggctgtggtgactcagccactctcagtgtcagtggccctgggacagacggccaagattccctgtgggggaaacaacattggaaggaaaagtgtgcactggtaccagcagaagccaggccaggcccctgtgttggtcatttatagggataacaaccggccctctgggatccctgagcgattctctggctccaactcggggaacacggccaccctgaccatcagcagagcccaagccggggatgaggctgactattactgtcaggtgtgggacagcaacactggagtcttcggaactgggaccaaggtcaccgtccta

SEQ ID NO: 339 - DNA, 369 bp, Artificial sequence
  Synthetic antibody
gaagtgcagctggtggagtctgggggaggcttggtacagcctggcaggtccctgagactctcctgtgcagcccctcgattcacctttgatgattatgccatacactgggtccggcaggttccagggaagggcctggagtgggtctctggtagtagttggaatagtggtaccataggctatgcgccttctgtgaagggccgattcaccatttccagagacaacgccaagaactccctgtttctgcaaatgaacagtctgagagttgaggacacggccttatattactgtgcaatcctcccgggtgactacaaccgcgttgcggatgtttttgatatctggggccaggggacaatggtcatcgtctcctca

SEQ ID NO: 340 - DNA, 333 bp, Artificial sequence
  Synthetic antibody
caggctgtggtgacgcagccgccctcagtgtctggggccccagggcagagggtcaccatttcctgcactgggagcagctccaacatcggggcaggttatgatgtacactggtaccagcagcttccaggaacagcccccaaactcctcatctatggtaacaacaatcggccctcaggggtccctgaccgattctctggctccaagtctggcacctcagcctccctggccatcactgggctccaggttgaggacgaggctgattattactgccagtcctatgacagcagcctgagtggttatgtcttcggaactgggaccaaggtcaccgtccta

SEQ ID NO: 341 - DNA, 369 bp, Artificial sequence
  Synthetic antibody
caggtgcagctggtggagtctgggggaggcttggtacagcctggcaggtccctgagactctcctgtgcagcctctagattcacatttgatgatcacgccatgcactgggtccggcaagccccagggaagggcctggagtgggtctcaggtattagttggaatggtggtatcataggctatgcggactctgtgaagggccgattcaccatctccagagacaacgccaagaactccctgtatctgcaaatgaacagtctgagagctgaggacacggccttgtattactgtgcaaaagggatggggttcggggagtccaactactacgctatggacgtctggggccaagggacaatggtcaccgtctcctca

SEQ ID NO: 342 - DNA, 333 bp, Artificial sequence
  Synthetic antibody
cagtctgccctgactcagcctgcctccgtgtctgggtctcctggacagtcgatcaccatctcctgcactggaaccagcagtgacgttggtggttataattatgtctcctggtaccaacaacacccaggcaaagcccccaaactcattatttatgatgtcagtaaccggccctcaggggtttctaatcgcttctctggctccaagtctggcaacacggcctccctgaccatctctgggctccaggctgaggacgaggcttattattactgcagctcatatacaagcatcagcaatctcgtggtattcggcggagggaccaaggtcaccgtccta

SEQ ID NO: 343 - DNA, 372 bp, Artificial sequence
  Synthetic antibody
caggtgcagctacatcagtggggcgcaggactgttgaagccttcggagaccctgtcccgcacctgcgctgtctatggtgggtccttcagtggttactcctggacctggatccgccaggccccggggaaggggctggaatggattggggaagtcaatcatggtggaaaaaccaactataacccgtccctcgagagtcgactcagcatatcagttgacacgtccaagaaccagttctccctggaactgaggtctgtgaccgccgcggacacggctgtttattactgtgcgagaggctatgacaccaattggtatggtgacggatacaactggttcgacccctggggccagggaaccctggtcagcgtctcctca

SEQ ID NO: 344 - DNA, 321 bp, Artificial sequence
  Synthetic antibody
gacatccagatgacccagtctccatcttccctgtctgcatctgttggagacagagtcaccatcacttgccgggcaagtcagaaaattgacaactttttaaattggtatcaacagaagccagggaaagcccctaagctcctgatccatggtgcatccaatttgcaaggtggggtctcatccaggttcagtggcagtagatctgggacagatttcactctcaccatcaccaatctgcagcctgaagattttgcaacttactactgtcaacagagttacaataccccgatcaccttcggccaagggacacgactggagattaaa

SEQ ID NO: 345 - DNA, 372 bp, Artificial sequence
  Synthetic antibody
caggtgcagctacatcagtggggcgcaggactgttgaagccttcggagaccctgtcccgcacctgcgctgtctatggtgggtccttcagtggttactcctggacctggatccgccaggccccggggaaggggctggaatggattggcgaaatcaatcatggtggaagcaccaagtacaacccgtccctcgagagtcgagtcaccatatcagtagacacgtccaagaaacagttctccctggaattgaggtctgtgaccgccgccgacacggctgtttattactgtgcgagaggctatgacaccaactggtatggtgacggatacaactggttcgacccctggggccagggaaccctggtcagcgtctcctca

SEQ ID NO: 346 - DNA, 321 bp, Artificial sequence
  Synthetic antibody
aattttatgctgactcagccagcctcggtgtcagtggccccaggaaagacggccagcattacctgtgggggtgacaacattggaactaaaagtgtgcactggtaccgccagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgggcgatagtagtagtcatgtggtattcggcggagggaccaaggtcaccgtccta

SEQ ID NO: 347 - DNA, 40 bp, Artificial sequence
  Synthetic primer
acaggtgtccactcgctagctagagtgcagcctaccgaga

SEQ ID NO: 348 - DNA, 33 bp, Artificial sequence
  Synthetic primer
agcaacaacctggacagccgggtcggcggcaac

SEQ ID NO: 349 - DNA, 33 bp, Artificial sequence
  Synthetic primer
agcaacaacctggacagctccgtcggcggcaac

SEQ ID NO: 350 - DNA, 39 bp, Artificial sequence
  Synthetic primer
accccttgcaatggcgtggccggcttcaactgctacttc

SEQ ID NO: 351 - DNA, 39 bp, Artificial sequence
  Synthetic primer
tgcaatggcgtggaaggctccaactgctacttcccactg

SEQ ID NO: 352 - DNA, 39 bp, Artificial sequence
  Synthetic primer
tacggcttccagcctacagccggcgtgggctaccagcct

SEQ ID NO: 353 - DNA, 20 bp, Artificial sequence
  Synthetic primer
gaaatacctgctgccgactg

SEQ ID NO: 354 - DNA, 23 bp, Artificial sequence
  Synthetic primer
tgttatttctagtgatgttcttg

SEQ ID NO: 355 - DNA, 18 bp, Artificial sequence
  Synthetic primer
tggaccttgaaggaaaac

SEQ ID NO: 356 - DNA, 22 bp, Artificial sequence
  Synthetic primer
tccacttttaagtgttatggag

SEQ ID NO: 357 - DNA, 18 bp, Artificial sequence
  Synthetic primer
aggcacaggtgttcttac

SEQ ID NO: 358 - DNA, 19 bp, Artificial sequence
  Synthetic primer
gtacacaattaaaccgtgc

SEQ ID NO: 359 - DNA, 18 bp, Artificial sequence
  Synthetic primer
cacaagctttaaacacgc

SEQ ID NO: 360 - DNA, 19 bp, Artificial sequence
  Synthetic primer
tctctggcattaatgcttc

SEQ ID NO: 361 - skipped

SEQ ID NO: 362 - DNA, 41 bp, Artificial sequence
  Synthetic primer
ctaacaatcttgattctagggttggtggtaattataattac

SEQ ID NO: 363 - DNA, 31 bp, Artificial sequence
  Synthetic primer
gttgcaggttttaattgttactttcctttac

SEQ ID NO: 364 - DNA, 40 bp, Artificial sequence
  Synthetic primer
gtgaaccgcctccacctgcgcagaagttcacgcatttgtt

SEQ ID NO: 365 - DNA, 33 bp, Artificial sequence
  Synthetic primer
gttgccgccgacccggctgtccaggttgttgct

SEQ ID NO: 366 - DNA, 33 bp, Artificial sequence
  Synthetic primer
gttgccgccgacggagctgtccaggttgttgct

SEQ ID NO: 367 - DNA, 39 bp, Artificial sequence
  Synthetic primer
gaagtagcagttgaagccggccacgccattgcaaggggt

SEQ ID NO: 368 - DNA, 39 bp, Artificial sequence
  Synthetic primer
cagtgggaagtagcagttggagccttccacgccattgca

SEQ ID NO: 369 - DNA, 39 bp, Artificial sequence
  Synthetic primer
aggctggtagcccacgccggctgtaggctggaagccgta

SEQ ID NO: 370 - DNA, 22 bp, Artificial sequence
  Synthetic primer
caatcaagccagctataaaacc

SEQ ID NO: 371 - DNA, 27 bp, Artificial sequence
  Synthetic primer
caccaaccctagaatcaagattgttag

SEQ ID NO: 372 - DNA, 40 bp, Artificial sequence
  Synthetic primer
ggaaagtaacaattaaaacctgcaacaccattacaaggtg

SEQ ID NO: 373 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 374 - protein, 222 aa, Severe acute respiratory syndrome coronavirus
RVVPSRDVVRFPNITNLCPFGEVFNATKFPSVYAWERKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSYELLNAPATVCGPKLSTDLIKNQCVNF

SEQ ID NO: 375 - protein, 7 aa, Artificial sequence
  Synthetic protein
KVGGNYN

SEQ ID NO: 376 - protein, 7 aa, Artificial sequence
  Synthetic protein
AVGGNYN

SEQ ID NO: 377 - protein, 7 aa, Artificial sequence
  Synthetic protein
KAGGNYN

SEQ ID NO: 378 - protein, 7 aa, Artificial sequence
  Synthetic protein
KVAGNYN

SEQ ID NO: 379 - protein, 7 aa, Artificial sequence
  Synthetic protein
KVGANYN

SEQ ID NO: 380 - protein, 7 aa, Artificial sequence
  Synthetic protein
KVGGAYN

SEQ ID NO: 381 - protein, 7 aa, Artificial sequence
  Synthetic protein
KVGGNAN

SEQ ID NO: 382 - protein, 7 aa, Artificial sequence
  Synthetic protein
KVGGNYA

SEQ ID NO: 383 - protein, 51 aa, Artificial sequence
  Synthetic protein
NLDSKVGGNYNYLYRLFRKSNLKPFERDISTEIYQAGSTPCNGVEGFNCYF

SEQ ID NO: 384 - protein, 51 aa, Artificial sequence
  Synthetic protein
NLDSRVGGNYNYLYRLFRKSNLKPFERDISTEIYQAGSTPCNGVEGFNCYF

SEQ ID NO: 385 - protein, 51 aa, Artificial sequence
  Synthetic protein
  misc_feature (7)..(9): Xaa can be any naturally occurring amino acid
NLDSKVXXXYNYLYRLFRKSNLKPFERDISTEIYQAGSTPCNGVAGFNCYF

SEQ ID NO: 386 - protein, 51 aa, Artificial sequence
  Synthetic protein
NLDSKVGGNYNYLYRLFRKSNLKPFERDISTEIYQAGSTPCNGVKGFNCYF

SEQ ID NO: 387 - protein, 51 aa, Artificial sequence; identical to SEQ ID NO: 386
  Synthetic protein

SEQ ID NO: 388 - protein, 51 aa, Artificial sequence; identical to SEQ ID NO: 386
  Synthetic protein

SEQ ID NO: 389 - protein, 51 aa, Artificial sequence; identical to SEQ ID NO: 386
  Synthetic protein

SEQ ID NO: 390 - protein, 51 aa, Artificial sequence; identical to SEQ ID NO: 386
  Synthetic protein

SEQ ID NO: 391 - protein, 51 aa, Artificial sequence; identical to SEQ ID NO: 386
  Synthetic protein

SEQ ID NO: 392 - protein, 51 aa, Artificial sequence
  Synthetic protein
NLDSKVGGNYNYLYRLFRKSNLKPFERDISTEIYQAGSTPCNGVQGFNCYF

SEQ ID NO: 393 - protein, 51 aa, Artificial sequence; identical to SEQ ID NO: 392
  Synthetic protein

SEQ ID NO: 394 - protein, 51 aa, Artificial sequence; identical to SEQ ID NO: 392
  Synthetic protein

SEQ ID NO: 395 - protein, 51 aa, Artificial sequence; identical to SEQ ID NO: 392
  Synthetic protein

SEQ ID NO: 396 - protein, 51 aa, Artificial sequence; identical to SEQ ID NO: 392
  Synthetic protein

SEQ ID NO: 397 - protein, 51 aa, Artificial sequence
  Synthetic protein
NLDSKVGGNYNYLYRLFRKSNLKPFERDISTEIYQAGSTPCNGVDGFNCYF

SEQ ID NO: 398 - protein, 51 aa, Artificial sequence; identical to SEQ ID NO: 397
  Synthetic protein

SEQ ID NO: 399 - protein, 20 aa, Severe acute respiratory syndrome coronavirus 2
GVSPTKLNDLCFTNVYADSF

SEQ ID NO: 400 - protein, 20 aa, Severe acute respiratory syndrome coronavirus
GVSATKLNDLCFSNVYADSF

SEQ ID NO: 401 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 402 - protein, 1255 aa, SARS coronavirus
MFIFLLFLTLTSGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFLPAQDTWGTSAAAYFVGYLKPTTFMLKYDENGTITDAVDCSQNPLAELKCSVKSFEIDKGIYQTSNFRVVPSRDVVRFPNITNLCPFGEVFNATKFPSVYAWERKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSYELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKDFGGFNFSQILPDPLKSTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

(19) United States
(12) Patent Application Publication
(10) Pub. No.: US 2023/0399384 A1
(43) Pub. Date: Dec. 14, 2023

(54) ANTIBODIES TO CORONAVIRUS SPIKE PROTEIN AND METHODS OF USE THEREOF

(71) Applicant: THE BOARD OF REGENTS OF THE UNIVERSITY OF TEXAS SYSTEM, Austin, TX (US)

(72) Inventors: Zhiqiang KU, Houston, TX (US); Ningyan ZHANG, Houston, TX (US); Zhiqiang AN, Houston, TX (US); Xuping XIE, Galveston, TX (US); Pei-Yong SHI, Galveston, TX (US)

(73) Assignee: THE BOARD OF REGENTS OF THE UNIVERSITY OF TEXAS SYSTEM, Austin, TX (US)

(21) Appl. No.: 17/996,874

(22) PCT Filed: Apr. 22, 2021
(86) PCT No.: PCT/US2021/028646

§ 371 Date: Oct. 21, 2022

Related U.S. Application Data
(60) Provisional application No. 63/015,257, filed on Apr. 24, 2020.

(51) Int. Cl. C07K 16/10; A61P 31/14

(52) U.S. Cl. CPC C07K 16/1003; A61P 31/14; C07K 2317/76; C07K 2317/92; C07K 2317/34; A61K 2039/505

(57) ABSTRACT

Provided herein are antibodies binding to Coronavirus S protein and the uses of the antibodies in detecting and treating Coronavirus infection, such as COVID-19.

### PRIORITY CLAIM

This application claims benefit of priority to U.S. Provisional Application Ser. No. 63/015,257, filed Apr. 24, 2020, the entire contents of which are hereby incorporated by reference.

### BACKGROUND

### 1. Field

The present disclosure relates generally to the fields of medicine, virology, and immunology. More particular, the disclosure relates to antibodies that bind to Coronavirus S protein that can be used to detect and treat coronavirus infection.

### 2. Description of Related Art

An outbreak of COVID-19, the disease caused by infection of the coronavirus SARS-CoV-2, began in December 2019 in China has resulted in millions of infections and more than 100 thousand deaths. Like the virus that caused the SARS outbreak several years prior, SARS-CoV, the SARS-CoV-2 virus use their spike proteins to bind host cellular receptor angiotensin-converting enzyme 2 (ACE2). The interaction between the receptor binding domain (RBD) of the spike glycoprotein and the full-length human ACE2 protein. Although the sequence of the SARS-CoV-2 spike protein is a know there remains a need for antibodies that bind to the spike protein and that could be used to detect and treat infection.

### SUMMARY

In some embodiments, the present disclosure provides isolated monoclonal antibodies or an antigen-binding fragment thereof comprising cloned paired heavy and light chain CDRs from Table A or Table B. In some aspects, the antibody or fragment thereof is encoded by clone-paired heavy and light chain sequences from FIGS. 13 and 15, respectively. In some aspects, the antibody or fragment thereof is encoded by heavy and light chain variable sequences having at least 70%, 80%, 90% or 95% identity to clone-paired sequences from FIGS. 14 and 16, respectively. In some aspects, the isolated monoclonal antibody is a murine, a rodent, or a rabbit. In some aspects, the isolated monoclonal antibody is a humanized, or human antibody. In some aspects, the antigen-binding fragment is a recombinant ScFv (single chain fragment variable) antibody, Fab fragment, F(ab′)2 fragment, or Fv fragment. In some aspects, the isolated monoclonal antibody is a bispecific antibody or a chimeric antibody. In some aspects, said antibody is an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

In some aspects, the antibody binds to a coronavirus spike (S) protein. In further aspects, the antibody binds to the SARS-CoV-2 S protein. In still further aspects, the antibody binds to the RBD domain (319-541) of the SARS-CoV-2 S protein. In some aspects, the antibody binds to the SARS-CoV S protein. In some aspects, the antibody binds to the RBD domain (306-527) of the SARS-CoV S protein. In some aspects, the antibody binds to the SARS-CoV S protein and the SARS-CoV-2 S protein. In some aspects, the antibody is a virus neutralizing antibody. In further aspects, the antibody exhibits a neutralization activity (effective concentration 50; EC₅₀) of less than 20, 10 or 5 (μg/ml). In still further aspects, the antibody exhibits a neutralization activity EC₅₀ of about 0.1 to 20 (μg/ml). In some aspects, the antibody is a virus neutralizing antibody. In further aspects, the antibody is a SARS-CoV neutralizing antibody. In some aspects, the antibody is a SARS-CoV-2 neutralizing antibody. In some aspects, the antibody is a SARS-CoV and SARS-CoV-2 neutralizing antibody.

In other embodiments, the present disclosure provides isolated monoclonal antibodies or an antigen binding fragment thereof, which competes for the same epitope with the isolated monoclonal antibody or an antigen-binding fragment thereof of the present disclosure. In still other embodiments, the present disclosure provides pharmaceutical compositions comprising the isolated monoclonal antibody or an antigen-binding fragment thereof of the present disclosure, and a pharmaceutically acceptable carrier. In yet other embodiments, the present disclosure provides isolated nucleic acids that encode the isolated monoclonal antibodies of the present disclosure. In other embodiments, the present disclosure provides vectors comprising the isolated nucleic acids of the present disclosure. In still other embodiments, the present disclosure provides host cells comprising the vectors of the present disclosure. In some aspects, the host cell is a mammalian cell. In some aspects, the host cell is a CHO cell. In yet other embodiments, the present disclosure provides hybridomas encoding or producing the isolated monoclonal antibodies of the present disclosure. In other embodiments, the present disclosure provides processes of producing an antibody, comprising culturing host cells of the present disclosure under conditions suitable for expressing the antibody, and recovering the antibody. In still other embodiments, the present disclosure provides chimeric antigen receptor (CAR) proteins comprising an antigen-binding fragment of the present disclosure. In yet other embodiments, the present disclosure provides isolated nucleic acids that encodes a CAR protein of the present disclosure. In other embodiments, the present disclosure provides vectors comprising an isolated nucleic acid of the present disclosure. In still other embodiments, the present disclosure provides engineered cells comprising an isolated nucleic acid of the present disclosure. In some aspects, the cell is a T cell, NK cell, or macrophage.

In yet other embodiments, the present disclosure provides methods of treating or ameliorating a Coronavirus infection in a subject, the method comprising administering to the subject a therapeutically effective amount of the antibody or an antigen-binding fragment thereof of the present disclosure or the engineered cell of the present disclosure. In some aspects, the method reduces viral replication in the subject. In some aspects, the method reduces inflammation in the lungs of a subject. In some aspects, the subject is infected with SARS-CoV. In some aspects, the subject is infected with SARS-CoV-2. In some aspects, the subject has pneumonia. In some aspects, the subject is on a respirator or oxygen supplementation. In some aspects, the antibody or an antigen-binding fragment thereof is administered intravenously, intra-arterially, subcutaneously or via inhalation. In some aspects, the methods further comprise administering to the subject a second anti-viral therapy.

In other embodiments, the present disclosure provides methods of detecting coronavirus, coronavirus S protein and/or coronavirus-infected cells in a sample or subject comprising: (a) contacting a subject or a sample from the subject with the antibody or an antigen-binding fragment thereof of the present disclosure; and (b) detecting binding of said antibody to a cancer cell or cancer stem cell in said subject or sample. In some aspects, the sample is a body fluid or biopsy. In some aspects, the sample is blood, bone marrow, sputum, tears, saliva, mucous, serum, urine, feces or a nasal swab. In some aspects, detection comprises immunohistochemistry, flow cytometry, FACS, ELISA, RIA or Western blot. In some aspects, the methods further comprise performing steps (a) and (b) a second time and determining a change in detection levels as compared to the first time. In some aspects, said isolated monoclonal antibody or an antigen binding fragment thereof further comprises a label. In further aspects, said label is a peptide tag, an enzyme, a magnetic particle, a chromophore, a fluorescent molecule, a chemo-luminescent molecule, or a dye. In some aspects, said isolated monoclonal antibody or an antigen binding fragment thereof is conjugated to a liposome or nanoparticle.

In still other embodiments, the present disclosure provides isolated monoclonal antibodies or an antigen-binding fragment thereof wherein said antibody binds to the RGB domain (319-541) of SAR-CoV-2 and exhibits SAR-CoV-2 neutralizing activity. In some aspects, the antibody exhibits a neutralization activity (effective concentration 50; EC₅₀) of less than 20, 10 or 5 (μg/ml). In further aspects, the antibody exhibits a neutralization activity of EC₅₀ of about 0.1 to 20 (μg/ml). In some aspects, the antibody also binds to the SARS-CoV S protein. In further aspects, the antibody binds to the RBD domain (306-527) of the SARS-CoV S protein. In some aspects, the antibody exhibits neutralizing activity of SARS-CoV. In some aspects, isolated monoclonal antibodies or an antigen-binding fragment thereof comprise cloned paired heavy and light chain CDRs from Table A or Table B. In further aspects, the antibody or fragment thereof is encoded by clone-paired heavy and light chain sequences from FIGS. 13 and 15, respectively. In some aspects, antibody or fragment thereof is encoded by heavy and light chain variable sequences having at least 70%, 80%, 90% or 95% identity to clone-paired sequences from FIGS. 14 and 16, respectively. In some aspects, the isolated monoclonal antibody is a murine, a rodent, or a rabbit. In some aspects, the isolated monoclonal antibody is a humanized, or human antibody. In some aspects, the antigen-binding fragment is a recombinant ScFv (single chain fragment variable) antibody, Fab fragment, F(ab′)2 fragment, or Fv fragment. In some aspects, the isolated monoclonal antibody is a bispecific antibody or a chimeric antibody. In some aspects, said antibody is an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern. In yet other embodiments, the present disclosure provides isolated monoclonal antibodies or an antigen binding fragment thereof, which competes for the same epitope with the isolated monoclonal antibody or an antigen-binding fragment thereof of the present disclosure.

In other embodiments, the present disclosure provides pharmaceutical compositions comprising an isolated monoclonal antibody or an antigen-binding fragment thereof of the present disclosure, and a pharmaceutically acceptable carrier.

The use of the word “a” or “an” when used in conjunction with the term “comprising” in the claims and/or the specification can mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain embodiments provided by the disclosure. The disclosed embodiments can be better understood by reference to one or more of these drawings in combination with the detailed description of specific embodiments presented herein.

FIGS. 1A-1F show phage panning to select antibodies specific to the RBD of SARS-CoV-2. FIG. 1A shows schematics of the structures of the SARS-CoV-2 spike protein and the SARS-CoV-2 and SARS-CoV RBD-Fc fusion proteins (RBD numbering relative to SEQ ID NO: 401 and SEQ ID NO: 402, respectively). NTD: N-terminal domain, RBD: receptor-binding domain, SD1: subdomain 1, SD2: subdomain 2, FP: fusion peptide, HR1: heptad repeat 1, HR2: heptad repeat 2, TM: transmembrane region, IC: intracellular domain FIG. 1B shows an SDS PAGE of purified sCoV2-RBD-Fc and sCoV-RBD-Fc proteins. FIG. 1C shows a diagram of phage panning and antibody selection. FIG. 1D shows a flowchart of the antibody selection process. The numbers of phages or antibodies at each step as shown in the parentheses. FIG. 1E: Phage ELISA binding to sCoV2-RBD and sCoV-RBD by the 35 phage clones with unique scFv sequences. The dash line is 5× the OD₄₅₀ₙₘ of a control phage and as a cut off for selection of phage binders. FIG. 1F Blocking of sCoV2-RBD binding to ACE2 by the 35 unique phages. The dash line is 50% of the value of a control phage and is a cut off for selection of phage blockers.

FIGS. 2A-2H show binding and blocking characteristics of SARS-CoV-2 mAbs. FIG. 2A: ELISA titration of mAb binding to sCoV2-RBD. The binding EC₅₀s are shown in the parentheses. FIG. 2B: ELISA titration of CoV2-20 binding to sCoV2-RBD and sCoV-RBD. FIG. 2C: The affinities of the 14 mAbs to the RBD of SARS-CoV-2. The affinity value KD, association constant value Kon dissociation constant value Koff and the R² from kinetic curve fitting are shown. FIGS. 2D-2F: The kinetics and affinities of mAb CoV2-20 to the spike of (FIG. 2D) SARS-CoV-2, (FIG. 2E) SARS-CoV, and (FIG. 2F) MERS-CoV. FIG. 2G: Blocking of sCoV2-RBD binding to ACE2 by purified CoV2 mAbs in different concentrations. FIG. 2H: Blocking of sCoV2-RBD and sCoV-RBD binding to ACE2 by CoV2-20 in different concentrations.

FIGS. 2I-2J show human monoclonal antibodies (mAb) recognize RBD and spike protein of SARS-CoV-2 using ELISA method. Relative binding affinity of COV2-mAbs at a fixed antibody concentration of 4 μg/ml. Recombinantly expressed RBD (FIG. 2I) or spike (S) protein (FIG. 2J) of SARS-COV2 were coated at a high-binding 96-well plates at 2 μg/ml in PBS. Binding signals (OD₄₅₀ₙₘ) are indicated on Y-axis and X-axis indicates individual CoV2-mAbs. IgG1 serves as an isotype negative control. Triplicate assay (n=3) was conducted and error bars indicate standard deviation (SD).

FIGS. 3A-3C show epitope mapping of SARS-CoV-2 mAbs. (FIG. 3A) The structure complex of SARS-CoV2-RBD and ACE2. The RBD is highlighted in purple with the receptor binding interface highlighted in yellow, and the ACE2 is highlighted in green. The structure is analyzed and depicted using Pymol based on the PDB 2AJF. (FIG. 3B) Side view of the SARS-CoV2-RBD. Fourteen residues within the receptor binding motif that are mutated to Alanine are indicated by arrows, numbering of residues relative to SEQ ID NO: 401. Five residues that directly interact with ACE2 are underlined. (FIG. 3C) The heat map of single amino acid mutations (relative to SEQ ID NO: 401) on mAb binding to the RBD based on ELISA binding assay. Residues that decrease mAb binding are indicated by a gradient of red colors, and residues that increased mAb binding are by a gradient of blue colors.

FIG. 4 shows a table of the germline gene origins, V region identity and CDR length of SARS CoV-2 mAbs.

FIGS. 5A & 5B show kinetics and affinities of SARS-CoV2-RBD and SARS-CoV-RBD to ACE2.

FIG. 6 shows phage ELISA binding to sCoV2-RBD and sCoV-RBD by the 376 tested output phage clones.

FIGS. 7A-7H show octet competition assay to test phage or antibody blocking of sCoV2-RBD binding to human ACE2.

FIGS. 8A & 8B show expression and purification of SARS-CoV2 mAbs.

FIG. 9 shows the kinetic and affinity characterizations of SARS-CoV2.

FIG. 10A shows an alignment of the RBD regions of SARS-CoV2 and SARS-CoV (numbering from SEQ ID NOs 401 and 402, respectively).

FIG. 10B is a polyacrylamide gel showing expression and purification of sCoV2-RBD proteins with single residue mutations (numbering relative to SEQ ID NO: 401).

FIG. 11 shows antiviral curves used to calculate NC₅₀ for the five exemplary antibodies.

FIG. 12 shows Cov2-mAb neutralization potency measured by a cell-based SARS-Cov2 cell infection assay. Briefly, a reporter SARS-CoV-2 (engineered with a mNeonGreen reporter gene) was incubated with the respective CoV-2 antibodies (10 μg/ml) at 37′C for 1 hour. The mixtures of virus and antibody were used to infect Vero E6 cells. At 24 hours post infection, the cells were quantified for mNeonGreen reporter signals. Percentage of neutralization was calculated using the formula: (infection in isotype control—infection in Cov2-mAb)/Infection in isotype control×100. Data bars show the average of three replications and error bars indicate SD.

FIG. 13 shows the DNA sequences encoding the heavy chain variable regions of the provided SARS-CoV-2 antibodies.

FIG. 14 shows the amino acid sequences of the heavy chain variable regions of the provided SARS-CoV-2 antibodies.

FIG. 15 shows the DNA sequences encoding the light chain variable regions of the provided SARS-CoV-2 antibodies.

FIG. 16 shows the amino acid sequences of the light chain variable regions of the provided SARS-CoV-2 antibodies-amino acid sequences of light chain variable regions.

FIG. 17A-C show isolation of RBD-directed human mAbs with neutralizing activities against SARS-CoV-2. FIGS. 17A-B show ELISA binding of purified mAbs to the RBD proteins (FIG. 17A) and the S proteins (FIG. 17B) of SARS-CoV-2 and SARS-CoV. The dashed line is 2× the OD₄₅₀ₙₘ of a control IgG1 and as a cut-off for binders.

FIG. 17C shows neutralization of live SARS-CoV-2 by the antibodies at 10 μg/ml. The dashed line indicates a 75% neutralization. The stars indicate the 11 mAbs with neutralization above 75%. Error bars indicate SD of triplicates.

FIGS. 18A-G show identification of CoV2-06 and CoV2-14 as two neutralizing mAbs suitable for cocktail. FIG. 18A shows neutralization titration curves of the top five mAbs with 50% neutralization titer (NT₅₀) below 1 μg/ml. Each data point is the mean±SD of two replicates. FIGS. 18B-C shows kinetic binding curves of the top five mAbs to the RBD protein (FIG. 18B) and the prefusion S protein (FIG. 18C) of SARS-CoV-2. The vertical dashed lines indicate the separation of association and dissociation phases. FIG. 18D shows epitope binning of 15 mAbs by a BLI-based cross-competition assay. Antibodies grouped into different bins shown in different colors. The top five neutralizing mAbs are shown in red. “+” denotes that the 1^{st} antibody competes with the 2^{nd} antibody and “−” denotes that the 1^{st} antibody does not compete with the 2^{nd} antibody. FIG. 18E shows simultaneous binding of CoV2-06 and CoV2-14 on the sCoV2-RBD protein. FIG. 18F shows dose-dependent percent neutralization of SARS-CoV-2 by individual CoV2-06, CoV2-14 mAbs and a cocktail of the two mAbs. n=3 biologically independent cells.

FIG. 18G shows a plot of calculated log-scale CI values (y-axis) versus fractional effects (x-axis). CI value=1 indicates additive effect, <1 means synergism and >1 indicates antagonism. Error bars indicate SD of triplicates.

FIGS. 19A-F show molecular determinants on the RBD for CoV2-06 and CoV2-14 binding and the mechanism of neutralization. FIG. 19A shows a schematic diagram of the shotgun and high-throughput epitope mapping strategy. Representative Alanine scan mutations in the RBD region of SARS-CoV-2 S (corresponding to amino acids 444 to 450 of SEQ ID NO: 401) and the critical procedures for mapping are shown. (SEQ ID NOS: 375-382) FIG. 19B shows the residues corresponding to SEQ ID NO: 401 critical for CoV2-06 and CoV2-14 binding, which are shown as green and blue spheres, respectively, on a structure of RBD (PDB: 6M0J). The residues that make direct contact with ACE2 are boxed. FIG. 19C shows CoV2-06 or Cov2-14 binding to the sCoV2-RBD proteins with indicated mutations (relative to SEQ ID NO: 401). Error bars indicate SD of duplicates wells. FIG. 19D shows the critical residues for CoV2-06 and CoV2-14 at the interface of RBD-ACE2 complex (PDB: 6M0J). The arrows indicate the K353 and K31 residues in ACE2, which are two virus-binding hotspots. The dashed circles indicate the steric clash of the two mAbs and ACE2 in binding to the RBD. FIG. 19E shows dose-dependent blocking of RBD binding to ACE2 by CoV2-06 and Cov2-14. FIG. 19F shows the landscape of CoV2-06 and CoV2-14 epitopes on the trimeric S structure (PDB: 6VSB). The RBD in each monomer is outlined and colored in yellow. The CoV2-06 epitope is colored in green and the CoV2-14 epitope in blue. The dashed circle indicates a steric clash of CoV2-14 and an adjacent “open” RBD in binding to a “closed” RBD.

FIGS. 20A-F show molecular determinants on the RBD for binding by CoV2-26, CoV2-09 and VH3-53 like antibodies. FIGS. 20A-B show the residues critical for CoV2-26 (FIG. 20A) and CoV2-09 (FIG. 20B) binding, which are shown as magenta spheres on the RBD-ACE2 complex (PDB: 6M0J). The arrows indicate the K353 and K31 residues in ACE2, which are two virus-binding hotspots. The dashed circles indicate the clash of mAb and ACE2 in binding to the RBD. FIG. 20C shows dose-dependent blocking of RBD binding to ACE2 by the mAbs. FIG. 20D shows the residues critical for the VH3-53 antibody CC12.1, which are shown as blue spheres on the RBD-ACE2 complex (PDB: 6M0J). FIG. 20E shows comparison of the critical residues for the CoV2-09 and the CC12.1 antibody. FIG. 20F shows the RBD residues (relative to SEQ ID NO: 401) critical for binding of the indicated mAbs.

FIGS. 21A-D show that CoV2-06 and CoV2-14 cocktail prevents escape mutation of live SARS-CoV-2. FIG. 21A shows a schematic diagram for the procedures of evaluating SARS-CoV-2 escape mutation under individual or cocktail mAbs. Green dots represent cell clusters expressing the mNeonGreen due to viral infection. FIG. 21B shows the mutated RBD residue (relative to SEQ ID NO: 401), occurring frequency and mAb neutralization of the mutant viruses. ND, not determined; NA, not available. FIG. 21C shows ELISA binding curves of indicated mAb to wild-type (WT, SEQ ID NO: 401) or mutant sCoV2-RBD proteins with the indicated mutations relative to SEQ ID NO: 401. Data points are mean±SD of two replicates. FIG. 21D shows a summary of the key RBD residues (relative to SEQ ID NO: 401), the ability to inhibit mutant virus and the methods of identifying the critical residues for cocktail mAbs in this study and published studies.

FIGS. 22A-C show effects of single-site or double-site mutations on the RBD affinity to ACE2, the expression level and the folding stability of RBD. FIG. 22A shows the relative binding affinities of the sCoV2-RBD mutant proteins with the indicated mutations relative to SEQ ID NO: 401 for binding to ACE2. The Y-axis indicates the reversed value of K_{D} of mutants/WT. Data are mean±SD of the K_{D} values from fitting of five kinetic curves. Two-tailed Student's t-test. The distribution of data points are not available from the Octet Data Analysis software. FIG. 22B show the relative expressing levels of the sCoV2-RBD mutant proteins with the indicated mutations relative to SEQ ID NO: 401 as compared to wild-type (WT) protein. The Y-axis indicates the value of protein concentration of mutants/WT. Data are mean±SD of triplicate wells of transfection. Two-tailed Student's t-test. FIG. 22C show the size-exclusion chromatography (SEC) analysis of purified sCoV2-RBD mutant or wild-type proteins. The retention volume of proteins with indicated molecular weight are shown by arrowheads. The percentages of protein aggregates are shown.

FIGS. 23A-C show sequence analysis of SARS-CoV-2 isolates with natural mutations at the K444, E484 or F486 sites of the RBD, relative to the SARS-CoV-2 RBD sequence of SEQ ID NO: 401. FIG. 23A shows a summary of total numbers, accession ID, collection date and geographic locations for the clinical SARS-CoV-2 isolates with indicated mutations. A total of 70,943 viral genome sequences were queried from GISAID and analyzed. FIG. 23B shows an alignment of the RBD sequences of the mutant viruses with the reference Wuhan-Hu-1 strain. (SEQ ID NOS: 383-398, with numbering in the figure relative to SEQ ID NO: 401) FIG. 23C shows the frequency of the virus variants with single mutations of the K444, E484 and F486 residues of SEQ ID NO: 401, or simultaneous mutations of K444+E484 or K444+F486 residues in the total analyzed viral sequences.

FIGS. 24A-F show antibody protection of SARS-CoV-2 infection in mice. FIG. 24A shows a diagram showing the N501Y adapted mutation in the S protein RBD of the SARS-CoV-2 mouse-adapted strain (CMA-3). FIG. 24B shows ELISA binding of CoV2-06 and CoV2-14 to the WT sCoV2-RBD or the N501A mutant. Error bars indicate SD of duplicates wells. FIG. 24C shows a schematic diagram of prophylactic or therapeutic evaluations of the antibodies. FIG. 24D shows the infectious viral load in the lung of CoV-06 or CoV2-14 treated mice compared to that of isotype IgG1 treated mice. The dashed line indicates the limit of detection (LOD) of the assay. FIG. 24E shows the infectious viral load in the lung of mice with indicated treatment. The median levels of the lung viral load were shown as solid lines. N=5 mice. Ordinary one-way ANOVA with Sidak's multiple comparison test. FIG. 24F shows representative sequencing results of the RBD regions of the viruses harvested from each treatment groups. The amino acid residues critical for antibody binding (numbering relative to SEQ ID NO: 401) are indicated by inverted triangles.

FIG. 25 shows kinetic binding curves of the sCoV-2-RBD and the sCoV-RBD to ACE2.

FIGS. 26A-C show germline gene origins, V-region identities, and the length of CDRs for the variable heavy and light chains. FIG. 26A shows the germline gene classes for the variable heavy (VH) and the variable light chains (VK/L) of the SARS-CoV-2 antibodies. The numbers for each V gene used are indicated in the pie chart. FIG. 26B shows a comparison of the numbers of kappa light chain (VK) and lambda light chain (VL) used in the two groups of antibodies with NT₇₅<10 μg/ml and NT₇₅>10 μg/ml. FIG. 26C shows a comparison of the amino acid numbers of the heavy chain CDR3 (CDR-H3) and the light chain CDR3 (CDR-L3) in the two groups of SARS-CoV-2 antibodies with NT₇₅<10 μg/ml and NT₇₅>10 μg/ml. Two-tailed Student's t-test.

FIGS. 27A-E show neutralization, RBD binding and RBD/ACE2 blocking activities of additional mAbs. FIG. 27A shows neutralization titration of the six remaining mAbs of the 11 mAbs with neutralizations above 75% at 10 μg/ml. Error bars indicate SD of duplicates. FIG. 27B shows kinetic binding curves of the six mAbs to the RBD protein of SARS-CoV-2. FIG. 27C shows ELISA titration and determination of the 50% effective binding concentration (EC₅₀) of the 11 neutralizing antibodies to sCoV2-RBD. Each data point is the mean±SD of two replicates. FIG. 27D shows a summary of the binding affinities (K_{D}), association constant (Kₒₙ), dissociation constant (K_{dis}), the 50% inhibition concentration (IC₅₀) of receptor blocking, and the NT₅₀ of the 11 neutralizing antibodies. FIG. 27E shows the correlation between NT₅₀ and the affinity K_{D} and between NT₅₀ and the ELISA binding EC₅₀ for each of the 11 mAbs. Pearson correlations were performed using Graphpad prism 8.

FIGS. 28A-H show a conserved epitope determined by the SARS-CoV-2 neutralizing and SARS-CoV cross-reactive mAb CoV2-12. FIG. 28A shows ELISA titration and determination of the 50% effective binding concentration (EC₅₀) of CoV2-12 to indicated the RBD proteins SARS-CoV and SARS-CoV-2. Data points are mean±SD of two replicates. FIGS. 28B-D show kinetic binding curves of CoV2-12 to the S protein of SARS-CoV-2 (FIG. 28B), SARS-CoV (FIG. 28C) and MERS-CoV (FIG. 28D). The dashed lines indicate the separation of association and dissociation phases. FIG. 28E shows SARS-CoV-2 neutralization titration of CoV-12. Data points are mean±SD of two replicates. FIG. 28F shows the critical residues for CoV2-12 binding are shown as magenta spheres in the RBD structure and their locations relative to the epitope residues (colored in pale cyan) of the CR3022 mAb. FIG. 28G shows an alignment of the CoV2-12 binding residues on the RBD of SARS-CoV-2 (numbering relative to SEQ ID NO: 401) and SARS-CoV. (numbering relative to SEQ ID NO: 402) (SEQ ID NOS: 399-400) FIG. 28H shows a summary of the critical residues on the SARS-CoV-2 RBD (SEQ ID NO: 401) for CoV2-12 and the indicated cross-reactive mAbs.

FIGS. 29A-J show expression of sCoV2-RBD mutant proteins (mutations relative to SEQ ID NO: 401) and characterization of their affinities to ACE2. FIG. 29A shows SDS-PAGE and coomassie blue staining of the purified sCoV2-RBD proteins with indicated amino acid mutations. FIGS. 29B-J show kinetic binding curves of the sCoV2-RBD wild type or mutant proteins to human ACE2.

FIGS. 30A-C show validation of CoV2-06 neutralization against SARS-CoV-2 S pseudovirus and SARS-CoV-2 clinical isolate. FIG. 30A shows SARS-CoV-2 S pseudovirus neutralization assay with CoV2-06. The numbers of the red-fluorescent protein (RFP) foci were counted and their ratio relative to the control group without antibody were calculated and plotted as the Y-axis. Data points are mean±SD of triplicates. FIG. 30B shows percent neutralization of SARS-CoV-2 clinical isolate (USA/WA1/2020) infection as determined by visualizing the CPE of cells. The number of wells without CPE relative to the total 8 replicate wells shown on top of each bar. The dashed line indicates 50% of neutralization. FIG. 30C shows representative images of protected and infected cells. The CPE, including cell detachment and syncytium, are indicated by arrows.

### DESCRIPTION OF ILLUSTRATIVE EMBODIMENTS

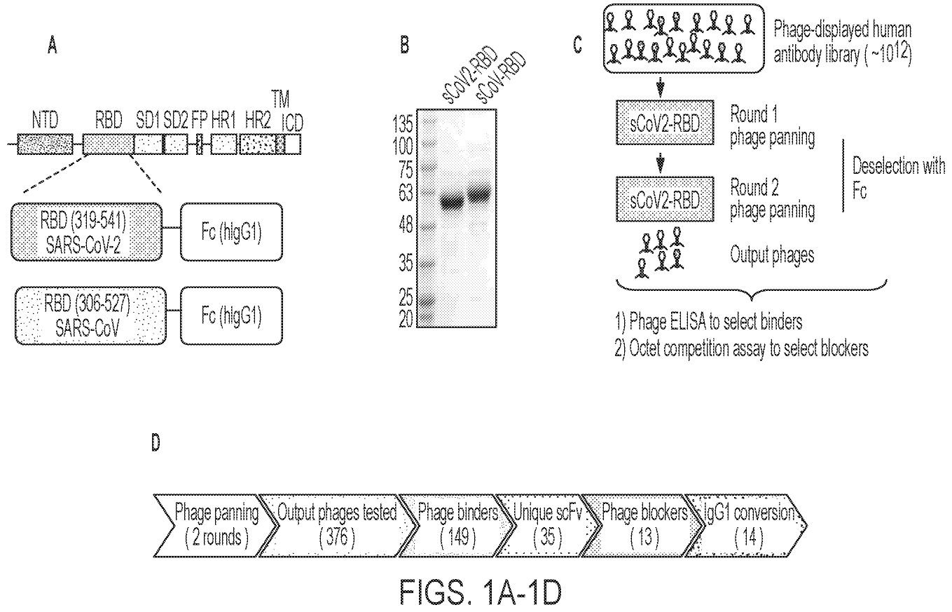

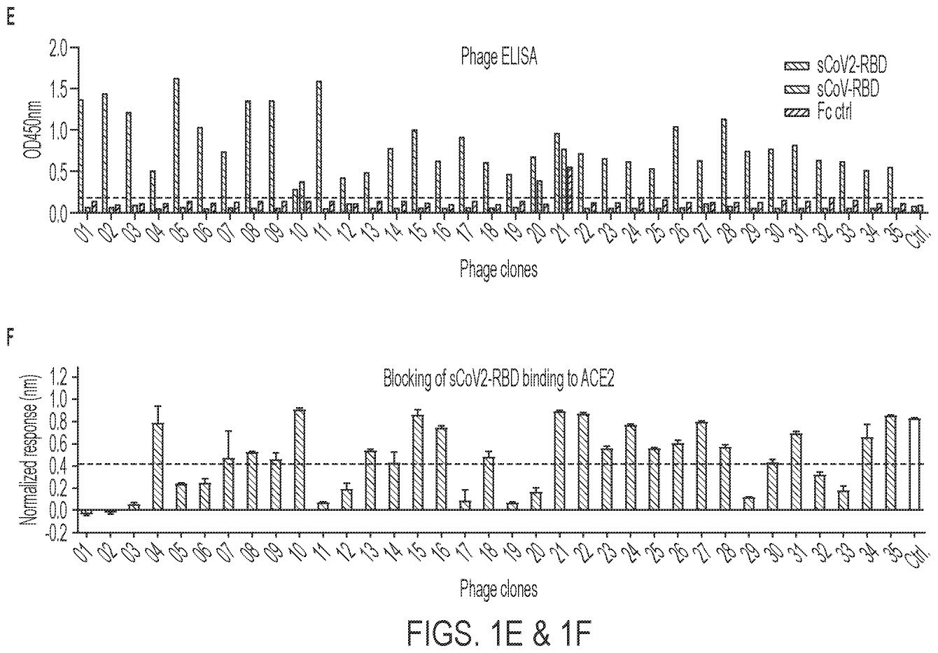

In certain aspects, there are provided coronavirus S protein-binding antibodies. Antibodies provided herein bind to the RBG of the SARS-CoV and/or SARS CoV-2 S protein and can exhibit neutralizing activity against the corona virus. For antibody identification, phage panning was used to select antibodies that inhibit the binding of the RBD of SARS-CoV-2 and which should interfere with virus ability to bind ACE2 (see, FIG. 1). The binding and blocking characteristics of the identified monoclonal antibodies was tested using ELISA titration to demonstrate the binding of the RBD of SARS-CoV-2 and EC₅₀ values were determined. The affinities of the mAbs to the RBD of SARS-CoV-2 were also determined. The affinity value KD, association constant value Kₒₙ, dissociation constant value K_{off} and the R2 from kinetic curve fitting were determined. At least one antibody bound significantly to both the RBD of SARS-CoV2 and that of SARS-CoV. The kinetics and affinities of this mAb for the spike proteins of SARS-CoV-2, SARS-CoV and MERS-CoV were characterized. The ability of sCoV2-RBD binding to ACE2 by purified sCoV2-RBD binding to ACE2 by purified CoV2 mAbs was demonstrated at different concentrations, as was the blocking of sCoV2-RBD and sCoV-RBD binding to ACE2 by the cross reactive monoclonal at different concentrations.

Antibodies identified and provided herein demonstrate significant neutralizing activity to SARS-CoV-2 and thus could be used in therapeutic application to reduce viral entry and/or replication and treat virus-associated lung damage. Likewise, the antibodies provided here can be used to detect the presence of virus and viral proteins in samples of interest.

In addition to single antibody therapies, antibody cocktail approaches have shown promise in avoiding neutralization escape by viruses in vitro (Wang et al., 2018; ter Meulen et al., 2006). An antibody cocktail for treating the Ebola virus disease has demonstrated clinic success (Levine, M., 2019). A dual-antibody cocktail (REGN10987 (imdevimab)+REGN10933 (casirivimab)) for SARS-CoV-2 has entered phase 2/3 clinical trials. This antibody cocktail is capable of preventing mutational escape as evaluated in cell culture using the VSV-SARS-CoV-2 S recombinant virus (Baum et al., 2020). Another antibody cocktail COV2-2130+COV2-2196, which exhibited neutralization synergy and animal protection (Zost et al., 2020b), has entered phase 1 clinical trial (NCT04507256). Other antibody cocktails, including BD-368-2+BD-629 (Du et al., 2020) and B38+H4 (Wu et al., 2020) have also been evaluated for neutralization activities. However, the molecular determinants optimal for cocktail mAbs and the mechanism of preventing viral escape remain poorly understood. These key challenges impede the development of mAb cocktails for SARS-CoV-2. Only certain mAb combinations can effectively prevent viral escape (Baum et al., 2020). This result suggests the importance of individual mAbs in a combination targeting different vulnerable sites. Therefore, identifying effective mAb cocktails, defining the molecular determinants on the RBD, and elucidating the mechanism of preventing viral escape are critically important to accelerate the development of effective cocktail mAb therapies for COVID-19.

Herein, the inventors show a cocktail of two mAbs (CoV2-06+CoV2-14) that target the RBD and cooperate with each other to prevent escape mutations. The two mAbs bind to non-overlapping epitopes of the RBD and independently block RBD and ACE2 interaction. The cocktail prevents SARS-CoV-2 escape mutations through a mechanism of imposing stronger mutational constraints on the RBD than individual mAbs. Individual mAbs and the cocktail confer protections against SARS-CoV-2 infection in mice. Overall, this comprehensive study provides important molecular insights for the development of antibody cocktail therapies for COVID-19.

### I. Definitions

It is to be understood that both the foregoing general description and the following detailed description are exemplary and explanatory only and are not restrictive of the claimed embodiments. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that can vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2nd ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989). Antibody variable domains can also be analyzed, e.g., using the IMGT information system (imgt.cines.fr (visited Jan. 24, 2021)) (IMGT®/V-Quest) to identify variable region segments, including CDRs. See, e.g., Brochet et al., Nucl. Acids Res. 36: W503-508, 2008. IMGT uses a different numbering system than Kabat. See, e.g., Lefranc, M.-P. et al., Dev. Comp. Immunol. 27:55-77 (2003). Correspondences are listed, for example, at imgt.org/IMGTScientificChart/Numbering/CDR1-IMGTgaps.html (visited Jan. 24, 2021). Unless otherwise specified, the CDRs as set forth herein were identified using IMGT®/V-Quest.

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196: 901-17 (1986); Chothia et al., Nature, 342: 877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79: 315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itself. Antigens are usually proteins and polysaccharides, less frequently also lipids. As used herein, antigens also include immunogens and haptens.

An “antigen binding protein” (“ABP”) as used herein means any protein that binds a specified target antigen. In the instant application, the specified target antigen is the Coronavirus S protein or fragment thereof. “Antigen binding protein” includes but is not limited to antibodies and antigen-binding fragment thereof. Peptibodies are another example of antigen binding proteins.

The term “antigen-binding fragment” as used herein refers to a portion of a protein which is capable of binding specifically to an antigen. In certain embodiment, the antigen-binding fragment is derived from an antibody comprising one or more CDRs, or any other antibody fragment that binds to an antigen but does not comprise an intact native antibody structure. In certain embodiments, the antigen-binding fragment is not derived from an antibody but rather is derived from a receptor. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a single domain antibody (sdAb), a camelid antibody or a nanobody, a domain antibody, and a bivalent domain antibody. In certain embodiments, an antigen-binding fragment is capable of binding to the same antigen to which the parent antibody binds. In certain embodiments, an antigen-binding fragment can comprise one or more CDRs from a particular human antibody grafted to a framework region from one or more different human antibodies. In certain embodiments, the antigen-binding fragment is derived from a receptor and contains one or more mutations. In certain embodiments, the antigen-binding fragment does not bind to the natural ligand of the receptor from which the antigen-binding fragment is derived.

A “Fab fragment” comprises one light chain and the CH1 and variable regions of one heavy chain. The heavy chain of a Fab molecule cannot form a disulfide bond with another heavy chain molecule.

A “Fab′ fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule.

A “F(ab′) 2 fragment” contains two light chains and two heavy chains containing a portion of the constant region between the CH1 and CH2 domains, such that an interchain disulfide bond is formed between the two heavy chains. A F(ab′) 2 fragment thus is composed of two Fab′ fragments that are held together by a disulfide bond between the two heavy chains.

An “Fc” region comprises two heavy chain fragments comprising the CH1 and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody can target the same or different antigens.

A “bivalent antigen binding protein” or “bivalent antibody” comprises two antigen binding sites. In some instances, the two binding sites have the same antigen specificities. Bivalent antigen binding proteins and bivalent antibodies can be bispecific, see, infra. A bivalent antibody other than a “multispecific” or “multifunctional” antibody, in certain embodiments, typically is understood to have each of its binding sites identical.

A “multispecific antigen binding protein” or “multispecific antibody” is one that targets more than one antigen or epitope.

A “bispecific,” “dual-specific” or “bifunctional” antigen binding protein or antibody is a hybrid antigen binding protein or antibody, respectively, having two different antigen binding sites. Bispecific antigen binding proteins and antibodies are a species of multispecific antigen binding protein antibody and can be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553. The two binding sites of a bispecific antigen binding protein or antibody will bind to two different epitopes, which can reside on the same or different protein targets.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative and exemplary embodiments for measuring binding affinity are described in the following.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the Coronavirus S protein specific antibodies of the present disclosure are specific to SARS-CoV-2 S protein, but can cross-react with certain other Coronavirus S proteins, e.g., SARS-CoV. In some embodiments, the antibody that binds to Coronavirus S protein has a dissociation constant (Kd) of ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

The term “compete” when used in the context of antigen binding proteins (e.g., antibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen (e.g., Coronavirus S protein or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acid in length. Two antibodies can bind the same epitope within an antigen if they exhibit competitive binding for the antigen.

The term “chimeric antigen receptor” or “CAR” as used herein refers to an artificially constructed hybrid protein or polypeptide containing an antigen binding domain of an antibody (e.g., a single chain variable fragment (scFv)) linked to a domain or signaling, e.g., T-cell signaling or T-cell activation domains, that activates an immune cell, e.g., a T cell or a NK cell (see, e.g., Kershaw et al., supra, Eshhar et al., Proc. Natl. Acad. Sci. USA, 90(2): 720-724 (1993), and Sadelain et al., Curr. Opin. Immunol. 21(2): 215-223 (2009)). CARs are capable of redirecting the immune cell specificity and reactivity toward a selected target in a non-MHC-restricted manner, taking advantage of the antigen-binding properties of monoclonal antibodies. The non-MHC-restricted antigen recognition confers immune cells expressing CARs on the ability to recognize an antigen independent of antigen processing, thus bypassing a major mechanism of tumor escape. In addition, when expressed in T-cells, CARs advantageously do not dimerize with endogenous T-cell receptor (TCR) alpha and beta chains.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05% or below 0.01%. In certain embodiments the disclosure provides a composition in which no amount of the specified component can be detected with standard analytical methods.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) can be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Examples of parameters that can be employed in determining percent identity for polypeptides or nucleotide sequences using the GAP program can be found in Needleman et al., 1970, J. Mol. Biol. 48:443-453.

Certain alignment schemes for aligning two amino acid sequences can result in matching of only a short region of the two sequences, and this small aligned region can have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally-occurring amino acid and polymers. The terms “polypeptide” and “protein” specifically encompass Coronavirus S protein binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a CORONAVIRUS S PROTEIN-binding antibody, useful fragments include but are not limited to a CDR region, a variable domain of a heavy and/or light chain, a portion of an antibody chain or just its variable region including two CDRs, and the like.

The pharmaceutically acceptable carriers useful in this provided in this disclosure are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat viral infection.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector can include nucleic acid sequences that permit it to replicate in the host cell, such as an origin of replication. A vector can also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. Coronavirus S Protein

The spike protein of SARS-CoV-2 plays an essential role in virus entry into host cells and thus a primary target by neutralizing antibodies 14, 33. The spike protein comprises an N-terminal S1 subunit and a C-terminal S2 subunit, which are responsible for receptor binding and membrane fusion, respectively 14. The S1 subunit is further divided into the N-terminal domain (NTD), the receptor-binding domain (RBD), the subdomain 1 (SD1) and subdomain 2 (SD2), and the S2 subunit is further divided into the fusion peptide (FP), the heptad repeat 1 (HR1) and heptad repeat 2 (HR2) 34. During coronavirus infection, the spike binds to a cellular receptor through its RBD, which triggers a conformational change of the spike 35. The activated spike is cleaved by a protease (eg. TMPRSS2 for SARS-CoV and SARS-CoV-2) at S1/S2 site to release the S1 subunit and expose the FP on S2 subunit 33. The HR1 and HR2 refold to the post-fusion conformation to drive membrane fusion 35. Due to the functionality and a higher immunogenicity of the S1, most neutralizing antibodies characterized for coronavirus to date target the S1 subunit, particularly the S1-RBD 35, 36. Two neutralizing mAbs (G2 and 7D10) were reported to target the S1-NTD region of MERS-CoV, and the two antibodies also blocked RBD interaction with the host receptor DPP4 37, 38. In comparison to the S1, which often elicit species-specific antibodies, the S2 has higher conservation and bears epitopes that could potentially be targeted by broad neutralizing antibodies 14, 37. An antibody with broad neutralizing activity against different coronaviruses, or at least SARS-related coronaviruses is of great value for confronting the next waves of coronavirus-related disease. However, broadly neutralizing antibodies against human coronaviruses were rare. A major challenge is that the S2 conformation is highly dynamic during membrane fusion, making it difficult to prepare the antigen for antibody discovery 37. Antigen stabilizing strategies used in HIV and RSV can be explored in the design of stable coronavirus S2 proteins.

A reference SARS-CoV-2 S protein (UniProtKB—P0DTC2 (SPIKE_SARS2)) is presented herein as SEQ ID NO: 401. Myriad variant SARS-CoV-2 S proteins have been sequenced and are available in the literature but share the common structure of SEQ ID NO: 401. The SARS-CoV-2 S protein RBD corresponds to amino acids 319 to 541 of SEQ ID NO: 401, underlined below (Yan, R. et al., Science 367:1444-1448 (2020)). As persons of ordinary skill in the art will recognize, the RBD of various SARS-CoV-2 S proteins present in the environment have mutated so an RBD that “corresponds” to amino acids 319 to 541 of SEQ ID NO: 401 may not be identical to amino acids 319 to 541 of SEQ ID NO: 401. The TMPRSS2 or furin cleavage site between the S1 and S2 subunits is between amino acids 685 and 686, and is indicated by a vertical line (Hoffmann, M. et al., Cell 181:271-280 (2020)).

```
SEQ ID NO: 401: SARS-COV-2 Spike Protein, UniProt: PODTC2
                10 20 30 40 50
MFVFLVLLPL VSSQCVNLTT RTQLPPAYTN SFTRGVYYPD KVFRSSVLHS
                60 70 80 90 100
TQDLFLPFFS NVTWFHAIHV SGTNGTKRFD NPVLPENDGV YFASTEKSNI
              110 120 130 140 150
IRGWIFGTTL DSKTQSLLIV NNATNVVIKV CEFQFCNDPF LGVYYHKNNK
              160 170 180 190 200
SWMESEFRVY SSANNCTFEY VSQPFLMDLE GKQGNFKNLR EFVFKNIDGY
              210 220 230 240 250
FKIYSKHTPI NLVRDLPQGF SALEPLVDLP IGINITRFQT LLALHRSYLT
              260 270 280 290 300
PGDSSSGWTA GAAAYYVGYL QPRTFLLKYN ENGTITDAVD CALDPLSETK
              310 320 330 340 350
CTLKSFTVEK GIYQTSNFRV QPTESIVRFP NITNLCPFGE VFNATRFASV
              360 370 380 390 400
YAWNRKRISN CVADYSVLYN SASFSTFKCY GVSPTKLNDL CFTNVYADSF
              410 420 430 440 450
VIRGDEVRQI APGQTGKIAD YNYKLPDDFT GCVIAWNSNN LDSKVGGNYN
              460 470 480 490 500
YLYRLFRKSN LKPFERDIST EIYQAGSTPC NGVEGENCYF PLQSYGFQPT
              510 520 530 540 550
NGVGYQPYRV VVLSFELLHA PATVCGPKKS TNLVKNKCVN FNENGLTGTG
              560 570 580 590 600
VLTESNKKFL PFQQFGRDIA DITDAVRDPQ TLEILDITPC SFGGVSVITP
              610 620 630 640 650
GTNTSNQVAV LYQDVNCTEV PVAIHADQLT PTWRVYSTGS NVFQTRAGCL
              660 670 680 690 700
IGAEHVNNSY ECDIPIGAGI CASYQTQTNS PRRARSVASQ SIIAYTMSLG
              710 720 730 740 750
AENSVAYSNN SIAIPTNFTI SVTTEILPVS MTKTSVDCTM YICGDSTECS
              760 770 780 790 800
NLLLQYGSFC TOLNRALTGI AVEQDKNTQE VFAQVKQIYK TPPIKDFGGF
              810 820 830 840 850
NFSQILPDPS KPSKRSFIED LLENKVTLAD AGFIKQYGDC LGDIAARDLI
              860 870 880 890 900
CAQKENGLTV LPPLLTDEMI AQYTSALLAG TITSGWTFGA GAALQIPFAM
              910 920 930 940 950
QMAYRFNGIG VTQNVLYENQ KLIANQFNSA IGKIQDSLSS TASALGKLQD
              960 970 980 990 1000
VVNQNAQALN TLVKQLSSNF GAISSVLNDI LSRLDKVEAE VQIDRLITGR
            1010 1020 1030 1040 1050
LOSLQTYVTQ QLIRAAEIRA SANLAATKMS ECVLGQSKRV DFCGKGYHLM
            1060 1070 1080 1090 1100
SFPQSAPHGV VFLHVTYVPA QEKNFTTAPA ICHDGKAHFP REGVFVSNGT
            1110 1120 1130 1140 1150
HWFVTQRNFY EPQIITTDNT FVSGNCDVVI GIVNNTVYDP LQPELDSFKE
            1160 1170 1180 1190 1200
ELDKYFKNHT SPDVDLGDIS GINASVVNIQ KEIDRLNEVA KNLNESLIDL
            1210 1220 1230 1240 1250
QELGKYEQYI KWPWYIWLGF IAGLIAIVMV TIMLCCMTSC CSCLKGCCSC
            1260 1270
GSCCKFDEDD SEPVLKGVKL HYT
```

A reference SARS-CoV S protein (GenBank: AAP51227.1) is presented herein as SEQ ID NO: 402. Myriad variant SARS-CoV S proteins have been sequenced and are available in the literature but share the common structure of SEQ ID NO: 402. The amino acids of the SARS-CoV S protein that correspond to the SARS-CoV-2 RBD correspond to amino acids 306 to 527 of SEQ ID NO: 402, underlined below. As persons of ordinary skill in the art will recognize, the RBD of various SARS-CoV S protein variants have been characterized so an RBD that “corresponds” to amino acids 306 to 527 of SEQ ID NO: 402 may not be identical to amino acids 306 to 527 of SEQ ID NO: 402.

SEQ ID NO: 402:
SARS-COV Spike Protein, GenBank: AAP51227.1

| | |
|---|---|
| 1 | MFIFLLFLTL TSGSDLDRCT TEDDVQAPNY |
| | TQHTSSMRGV YYPDEIFRSD TLYLTQDLFL |
| 61 | PFYSNVTGFH TINHTEDNPV IPFKDGIYFA |
| | ATEKSNVVRG WVEGSTMNNK SQSVIIINNS |
| 121 | TNVVIRACNF ELCDNPFFAV SKPMGTQTHT |
| | MIFDNAFNCT FEYISDAFSL DVSEKSGNEK |
| 181 | HLREFVFKNK DGFLYVYKGY QPIDVVRDLP |
| | SGENTLKPIF KLPLGINITN FRAILTAFLP |
| 241 | AQDTWGTSAA AYFVGYLKPT TEMLKYDENG |
| | TITDAVDCSQ NPLAELKCSV KSFEIDKGIY |
| 301 | QTSNFRVVPS RDVVRFPNIT NLCPFGEVEN |
| | ATKFPSVYAW ERKRISNCVA DYSVLYNSTE |
| 361 | FSTFKCYGVS ATKLNDLCFS NVYADSFVVK |
| | GDDVRQIAPG QTGVIADYNY KLPDDEMGCV |
| 421 | LAWNTRNIDA TSTGNYNYKY RYLRHGKLRP |
| | FERDISNVPF SPDGKPCTPP ALNCYWPLND |
| 481 | YGFYTTTGIG YQPYRVVVLS YELLNAPATV |
| | CGPKLSTDLI KNQCVNFNFN GLTGTGVLTP |
| 541 | SSKRFQPFQQ FGRDVSDFTD SVRDPKTSEI |
| | LDISPCSFGG VSVITPGINA SSEVAVLYQD |
| 601 | VNCTDVSTAI HADQLIPAWR IYSTGNNVFQ |
| | TQAGCLIGAE HVDTSYECDI PIGAGICASY |
| 661 | HTVSLLRSTS QKSIVAYTMS LGADSSIAYS |
| | NNTIAIPINF SISITTEVMP VSMAKTSVDC |
| 721 | NMYICGDSTE CANLLLQYGS FCTQLNRALS |
| | GIAAEQDRNT REVFAQVKQM YKTPTLKDEG |
| 781 | GFNFSQILPD PLKSTKRSFI EDLLENKVTL |
| | ADAGFMKQYG ECLGDINARD LICAQKENGL |
| 841 | TVLPPLLIDD MIAAYTAALV SGTATAGWTF |
| | GAGAALQIPF AMQMAYRENG IGVTQNVLYE |
| 901 | NQKQIANQFN KAISQIQESL TTISTALGKL |
| | QDVVNQNAQA LNTLVKQLSS NFGAISSVLN |
| 961 | DILSRLDKVE AEVQIDRLIT GRLQSLQTYV |
| | TQQLIRAAEI RASANLAATK MSECVLGQSK |
| 1021 | RVDFCGKGYH LMSFPQAAPH GVVFLHVTYV |
| | PSQERNETTA PAICHEGKAY FPREGVEVEN |
| 1081 | GTSWFITQRN FFSPQIITTD NTFVSGNCDV |
| | VIGIINNTVY DPLQPELDSF KEELDKYFKN |
| 1141 | HTSPDVDLGD ISGINASVVN IQKEIDRLNE |
| | VAKNLNESLI DLQELGKYEQ YIKWPWYVWL |
| 1201 | GFIAGLIAIV MVTILLCCMT SCCSCLKGAC |
| | SCGSCCKEDE DDSEPVLKGV KLHYT |

### III. Monoclonal Antibodies and Production Thereof

The monoclonal antibodies described herein can be prepared using standard methods, followed by screening, characterization and functional assessment. Variable regions can be sequenced and then subcloned into a human expression vector to produce the chimeric antibody genes, which are then expressed and purified. These chimeric antibodies can be tested for antigen binding, signaling blocking, and in xenograft experiments. Table A and Table B below provide the sequences of some of certain Coronavirus S protein-binding antibodies of the embodiments. In some cases, an antibody comprises a heavy chain comprising the three CDRs of a VH chain of Table A and a light chain comprising the three CDRs of the matching VL chain of Table A or an antibody comprises a heavy chain comprising the three CDRs of a VH chain of Table B and a light chain comprising the three CDRs of the matching VL chain of Table B. In some further aspects, an antibody comprises a heavy chain comprising the three CDRs of a VH chain of Table A and a light chain comprising the three CDRs of a different VL chain of Table A.

TABLE A
SARS-COV-2 antibodies-amino acid sequences of IGMT CDRs

| | SEQ | | SEQ | | SEQ | | | SEQ | | SEQ | | SEQ | |
|---|---|---|---|---|---|---|---|---|---|---|---|---|---|
| Heavy | ID | | ID | | ID | | Light | ID | | ID | | ID | |
| chain | NO | CDR1 | NO | CDR2 | NO | CDR3 | chain | NO | CDR1 | NO | CDR2 | NO | CDR3 |
| CoV2- | 1 | GYTFTNSY | 2 | INPISGGT | 3 | ARDRGDYDYGWGTSP | CoV2- | 4 | QSISRY | 5 | AAS | 6 | QQSFSPPIT |
| 01-HC | | | | | | FYFDY | 01-LC | | | | | | |
| CoV2- | 7 | GYTFTNSY | 8 | INPISGGT | 9 | ARDRGDYDYGWGTSP | CoV2- | 10 | QPISKY | 11 | AAS | 12 | QQSNGIPLT |
| 02-HC | | | | | | FYFDY | 02-LC | | | | | | |
| CoV2- | 13 | GYTFTNSY | 14 | INPISGGT | 15 | ARDRGDYDYGWGTSP | CoV2- | 16 | QSISSY | 17 | AAS | 18 | QQSYSTPLT |
| 03-HC | | | | | | FYFDY | 03-LC | | | | | | |
| CoV2- | 19 | GGSFSGYY | 20 | INHGGST | 21 | ARGYDTNWYGDGYNW | CoV2- | 22 | QSISRY | 23 | AAS | 24 | QQSFGTPLT |
| 04-HC | | | | | | FDP | 04-LC | | | | | | |
| CoV2- | 25 | GFTEDDYG | 26 | INWNGERI | 27 | ARP SGDYVAWYENL | CoV2- | 28 | GSDVGPYKY | 29 | DVN | 30 | GSYAGNNKWV |
| 05-HC | | | | | | | 05-LC | | | | | | |
| CoV2- | 31 | GGSISSNNW | 32 | IHHSGGT | 33 | TRDRAGGTYSGFDE | CoV2- | 34 | SSDVGGYNY | 35 | DVS | 36 | SSYTSSSTVV |
| 06-HC | | | | | | | 06-LC | | | | | | |
| CoV2- | 37 | GFTFSDFG | 38 | TSHDGSSK | 39 | AKDSDNGYDADFFDY | CoV2- | 40 | QSISSY | 41 | AAS | 42 | QQSYSTPLT |
| 07-HC | | | | | | | 07-LC | | | | | | |
| CoV2- | 43 | GGSFSGYY | 44 | INHGGST | 45 | ARGYDTNWYGDGYNW | CoV2- | 46 | SLRGSF | 47 | GIN | 48 | NSRESNSNRIL |
| 09-HC | | | | | | EDP | 09-LC | | | | | | |
| CoV2- | 49 | GFSFDNYA | 50 | ITGNSGTI | 51 | AKDTDYDSSGSYFDY | CoV2- | 52 | NIGRES | 53 | SDG | 54 | QVWDPDTDHYV |
| 11-HC | | | | | | | 11-LC | | | | | | |
| CoV2- | 55 | GFTFSSYA | 56 | VSDDGNMK | 57 | ARENYFWSGSIGGLDY | CoV2- | 58 | QSISSY | 59 | AAS | 60 | QQSYSTPGYT |
| 12-HC | | | | | | | 12-LC | | | | | | |
| CoV2- | 61 | GGNFRSHT | 62 | IMPREGAT | 63 | AADLGSGRKEDS | CoV2- | 64 | QGIGSD | 65 | AAS | 66 | LQHNSYPLT |
| 13-HC | | | | | | | 13-LC | | | | | | |
| CoV2- | 67 | GDSVSSNSAA | 68 | TYYRSKWYN | 69 | AREEQQLVHDYYYYG | CoV2- | 70 | SSDIGAYNY | 71 | EVS | 72 | SSYAGSIS |
| 14-HC | | | | | | MDV | 14-LC | | | | | | |
| CoV2- | 73 | GDSVSSNSAA | 74 | TYYRSKWYN | 75 | AREEQQLVHDYYYYG | CoV2- | 76 | SCTGISSDY | 77 | EVN | 78 | GSYAGSNTE |
| 15-HC | | | | | | MDV | 15-LC | | | | | | |
| CoV2- | 79 | GGSFSGYY | 80 | INHGGST | 81 | ARGYDTNWYGDGYNW | CoV2- | 82 | SSSIGSNT | 83 | NNN | 84 | QSYDTGLSGHV |
| 16-HC | | | | | | EDP | 16-LC | | | | | | |
| CoV2- | 85 | GFTFSSYS | 86 | ISSSSSYI | 87 | ARGNVDIVATGEVDA | CoV2- | 88 | SSTIGSNY | 89 | RNN | 90 | AAWDDSLSGYV |
| 17-HC | | | | | | FDI | 17-LC | | | | | | |
| CoV2- | 91 | GGTFSNYG | 92 | IIPIFGTA | 93 | ARDRDDALTGLGLGGG | CoV2- | 94 | TSNIGTNT | 95 | GND | 96 | AAWDERLNGYV |
| 18-HC | | | | | | FDI | 18-LC | | | | | | |
| CoV2- | 97 | GYTFTNSY | 98 | INPISGGT | 99 | ARDRGDYDYGWGTS | CoV2- | 100 | HTVNSY | 101 | AAS | 102 | QQSYRTPLT |
| 19-HC | | | | | | PFYFDY | 19-LC | | | | | | |
| CoV2- | 103 | GFIFDDYA | 104 | VNRDSTYV | 105 | VRGMTRVATDAFDE | CoV2- | 106 | QSINGY | 107 | SAS | 108 | QQSYSTPPVT |
| 20-HC | | | | | | | 20-LC | | | | | | |
| CoV2- | 109 | GYTFTNSY | 110 | INPISGGT | 111 | ARDRGDYDYGWGTSP | CoV2- | 112 | QSISSY | 113 | GAS | 114 | QQSYSIPFT |
| 22-HC | | | | | | FYFDY | 22-LC | | | | | | |
| CoV2- | 115 | GFTEDDYA | 116 | ISWNSGSI | 117 | AKDMDIWEGGGLDY | CoV2- | 118 | EGIGNW | 119 | EAS | 120 | LQANSFPIT |
| 23-HC | | | | | | | 23-LC | | | | | | |
| CoV2- | 121 | GGSFSGYY | 122 | INHGGST | 123 | ARGLVGGGAFDI | CoV2- | 124 | NIGSTG | 125 | DDT | 126 | QVWDSSGHSYV |
| 24-HC | | | | | | | 24-LC | | | | | | |
| CoV2- | 127 | GYTFTGYY | 128 | INPNSGGT | 129 | ARGYFDY | CoV2- | 130 | SSNIGSNT | 131 | SNN | 132 | ASWDDSLNGVV |
| 25-HC | | | | | | | 25-LC | | | | | | |
| CoV2- | 133 | GYTFTGYY | 134 | INPNSGGT | 135 | ARGYFDY | CoV2- | 136 | SSNIGSNS | 137 | AND | 138 | AAWDNSLKGVV |
| 26-HC | | | | | | | 26-LC | | | | | | |
| CoV2- | 139 | GGSFSGYY | 140 | INHGGST | 141 | ARGYDTNWYGDGYNW | CoV2- | 142 | SSNIGTNP | 143 | YNN | 144 | AAWDDSLKGWV |
| 27-HC | | | | | | FDP | 27-LC | | | | | | |
| CoV2- | 145 | GGTFSSYA | 146 | IIPIFGTA | 147 | ARDPYSGSYPGAFDI | CoV2- | 148 | SSDVGGYNY | 149 | DVS | 150 | NSYTRSSTSV |
| 28-HC | | | | | | | 28-LC | | | | | | |
| CoV2- | 151 | GFTFSSYW | 152 | IKQDGSEK | 153 | ARMADDDFWRDLPGFY | CoV2- | 154 | SSDVGGYNY | 155 | DVS | 156 | SSYTSSSTLV |
| 29-HC | | | | | | MDV | 29-LC | | | | | | |
| CoV2- | 157 | GGSFSGYS | 158 | INHGGST | 159 | ARGYDTNWYGDGYNW | CoV2- | 160 | NIGRKS | 161 | RDN | 162 | QVWDSNTGV |
| 31-HC | | | | | | FDP | 31-LC | | | | | | |
| CoV2- | 163 | RFTEDDYA | 164 | SSWNSGTI | 165 | AILPGDYNRVADVEDI | CoV2- | 166 | SSNIGAGYD | 167 | GNN | 168 | QSYDSSLSGYV |
| 32-HC | | | | | | | 32-LC | | | | | | |
| CoV2- | 169 | RFTEDDHA | 170 | ISWNGGII | 171 | AKGMGFGESNYYAMDV | CoV2- | 172 | SSDVGGYNY | 173 | DVS | 174 | SSYTSISNLVV |
| 33-HC | | | | | | | 33-LC | | | | | | |
| CoV2- | 175 | GGSFSGYS | 176 | VNHGGKT | 177 | ARGYDTNWYGDGYNWF | CoV2- | 178 | QKIDNE | 179 | GAS | 180 | QQSYNTPIT |
| 34-HC | | | | | | DP | 34-LC | | | | | | |
| CoV2- | 181 | GGSFSGYS | 182 | INHGGST | 183 | ARGYDTNWYGDGYNWF | CoV2- | 184 | NIGTKS | 185 | YDS | 186 | QVGDSSSHVV |
| 35-HC | | | | | | DP | 35-LC | | | | | | |

TABLE B
SARS-COV-2 antibodies-amino acid sequences of Kabat CDRs

| | SEQ | | SEQ | | SEQ | | SEQ | | SEQ | | SEQ | |
|---|---|---|---|---|---|---|---|---|---|---|---|---|
| | ID | | ID | CDR2 | ID | | ID | | ID | | ID | |
| Antibody | NO | CDR1 | NO | | NO | CDR3 | NO | CDR1 | NO | CDR2 | NO | CDR3 |
| CoV2- | 187 | GGSI | 188 | EIHH | 189 | DRAG | 190 | TGTS | 191 | DVSN | 192 | SSYT |
| 06 | | SSNN | | SGGT | | GTYS | | SDVG | | RPS | | SSST |
| | | WWT | | NYNP | | GEDE | | GYNY | | | | VV |
| | | | | SLKS | | | | VS | | | | |
| CoV2- | 193 | GGSF | 194 | EINH | 195 | GYDT | 196 | QGDS | 197 | GINN | 198 | NSRE |
| 09 | | SGYY | | GGST | | NWYG | | LRGS | | RPS | | SNSN |
| | | WT | | RYNP | | DGYN | | FAS | | | | RIL |
| | | | | SLES | | WEDP | | | | | | |
| CoV2- | 199 | GFTF | 200 | VVSD | 201 | ENYF | 202 | RASQ | 203 | AASS | 204 | QQSY |
| 12 | | SSYA | | DGNM | | WSGS | | SISS | | LQS | | STPG |
| | | MQ | | KFYA | | IGGL | | YLN | | | | YT |
| | | | | DSVK | | DY | | | | | | |
| | | | | G | | | | | | | | |
| CoV2- | 205 | GDSV | 206 | RTYY | 207 | EEQQ | 208 | TGTS | 209 | EVSN | 210 | SSYA |
| 14 | | SSNS | | RSKW | | LVHD | | SDIG | | RPS | | GSIS |
| | | AAWN | | YNDY | | YYYY | | AYNY | | | | |
| | | | | AVSV | | GMDV | | IS | | | | |
| | | | | KS | | | | | | | | |
| CoV2- | 211 | GGSF | 212 | EINH | 213 | GYDT | 214 | SGSS | 215 | EVSN | 216 | QSYD |
| 16 | | SGYY | | GGST | | NWYG | | SSIG | | RPS | | TGLS |
| | | WT | | RYNP | | DGYN | | SNTV | | | | GHV |
| | | | | SLES | | WEDP | | H | | | | |
| CoV2- | 217 | GYTF | 218 | RINP | 219 | GYFD | 220 | SGSS | 221 | ANDH | 222 | AAWD |
| 26 | | TGYY | | NSGG | | Y | | SNIG | | RPS | | NSLK |
| | | MH | | TNYA | | | | SNSV | | | | GVV |
| | | | | QKFQ | | | | N | | | | |
| | | | | G | | | | | | | | |

A. General Methods

It will be understood that monoclonal antibodies binding to Coronavirus S protein will have several applications. These include the production of diagnostic kits for use in detecting and diagnosing virus presence and viral infection. In these contexts, one can link such antibodies to diagnostic or therapeutic agents, use them as capture agents or competitors in competitive assays, or use them individually without additional agents being attached thereto. The antibodies can be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host. As is well known in the art, a given composition for immunization can vary in its immunogenicity. It is often necessary therefore to boost the host immune system, as can be achieved by coupling a peptide or polypeptide immunogen to a carrier. Exemplary carriers are keyhole limpet hemocyanin (KLH) and bovine serum albumin (BSA). Other albumins such as ovalbumin, mouse serum albumin or rabbit serum albumin can also be used as carriers. A polypeptide can be conjugated to a carrier protein through use of a variety of reagents, including, e.g., glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies can be monitored by sampling blood of the immunized animal at various points following immunization. A second, booster injection, also can be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells can be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures can be non-antibody-producing, have high fusion efficiency, and enzyme deficiencies that render then incapable of growing in certain selective media which support the growth of only the desired fused cells (hybridomas). Any one of a number of myeloma cells can be used, as are known to those of skill in the art (Goding, pp. 65-66, 1986; Campbell, pp. 75-83, 1984).

Methods for generating hybrids of antibody-producing spleen or lymph node cells and myeloma cells usually comprise mixing somatic cells with myeloma cells in a 2:1 proportion, though the proportion can vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary agents are aminopterin, methotrexate, and azaserine Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

In certain embodiments the selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines can be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by any of the methods disclosed herein can be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach can be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

1. Antibodies to Coronavirus S Protein

Antibodies or antigen-binding fragments thereof according to the present disclosure can be defined, in the first instance, by their binding specificity, which in this case is for Coronavirus S protein. Those of skill in the art, by assessing the binding specificity/affinity of a given antibody using techniques well known to those of skill in the art, can determine whether such antibodies fall within the scope of the instant claims.

In one aspect, there are provided antibodies and antigen-binding fragments that specifically bind to Coronavirus S protein. In some embodiments, when bound to Coronavirus S protein, such antibodies modulate the activation of Coronavirus S protein.

In some embodiments, the antibodies or antigen-binding fragments provided herein having clone-paired CDR's from the heavy chains and light chains illustrated in the tables below. Such antibodies can be produced by the clones discussed below in the Examples section using methods described herein. In certain embodiments, each CDR is defined in accordance with Kabat definition, the Chothia definition, the combination of Kabat definition and Chothia definition, the AbM definition, or the contact definition of CDR. In certain embodiments, the antibody or antigen-binding fragment is characterized by clone-paired heavy and light chain sequences from the tables below.

In certain embodiments, the antibodies can be defined by their variable sequence, which include additional “framework” regions. The antibody is characterized by clone-paired heavy chain and light chain amino acid sequences from the tables below. Furthermore, the antibodies sequences can vary from these sequences, particularly in regions outside the CDRs. For example, the amino acids can vary from those set out above by a given percentage, e.g., 80%, 85%, 90%, 91%, 92%, 93%, 94%, 95%, 96%, 97%, 98% or 99% homology, or the amino acids can vary from those set out above by permitting conservative substitutions (discussed below). Each of the foregoing apply to the amino acid sequences of the tables below. In another embodiment, the antibody derivatives of the present disclosure comprise VL and VH domains having up to 0, 1, 2, 3, 4, 5, 6, 7, 8, 9, 10 or more conservative or non-conservative amino acid substitutions, while still exhibiting the desired binding and functional properties.

While the antibodies of the present disclosure were generated as IgG's, it can be useful to modify the constant regions to alter their function. The constant regions of the antibodies typically mediate the binding of the antibody to host tissues or factors, including various cells of the immune system (e.g., effector cells) and the first component (Clq) of the classical complement system. Thus, the term “antibody” includes intact immunoglobulins of types IgA, IgG, IgE, IgD, IgM (as well as subtypes thereof), wherein the light chains of the immunoglobulin can be of types kappa or lambda. Within light and heavy chains, the variable and constant regions are joined by a 35 “J” region of about 12 or more amino acids, with the heavy chain also including a “D” region of about 10 more amino acids. See generally, Fundamental Immunology Ch. 7 (Paul, W., ed., 2^{nd} ed. Raven Press, N.Y. (1989).

The present disclosure further comprises nucleic acids which hybridize to nucleic acids encoding the antibodies disclosed herein. In general, the nucleic acids hybridize under moderate or high stringency conditions to nucleic acids that encode antibodies disclosed herein and also encode antibodies that maintain the ability to specifically bind to an Coronavirus S protein. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook et al., MOLECULAR CLONING: A LABORATORY MANUAL, 3 rd ed., Cold Spring Harbor Press, Cold Spring Harbor, N.Y. 2001). The conditions of temperature and ionic strength determine the “stringency” of the hybridization. Typical moderate stringency hybridization conditions are 40% formamide, with 5× or 6×SSC and 0.1% SDS at 42° C. High stringency hybridization conditions are 50% formamide, 5× or 6×SSC (0.15M NaCl and 0.015M Na-citrate) at 42° C. or, optionally, at a higher temperature (e.g., 57° C., 59° C., 60° C., 62° C., 63° C., 65° C. or 68° C.). Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids can hybridize. For hybrids of greater than 100 nucleotides in length, equations for calculating the melting temperature have been derived (see Sambrook et al., supra). For hybridization with shorter nucleic acids, e.g., oligonucleotides, the position of mismatches becomes more important, and the length of the oligonucleotide determines its specificity (see Sambrook et al., supra).

2. Exemplary Epitopes and Competing Antigen Binding Proteins

In another aspect, the present disclosure provides epitopes to which anti-Coronavirus S protein antibodies bind.

In some embodiments, epitopes that are bound by the antibodies described herein are useful. In certain embodiments, an epitope provided herein can be utilized to isolate antibodies or antigen binding proteins that bind to Coronavirus S protein. In certain embodiments, an epitope provided herein can be utilized to generate antibodies or antigen binding proteins which bind to Coronavirus S protein. In certain embodiments, an epitope or a sequence comprising an epitope provided herein can be utilized as an immunogen to generate antibodies or antigen binding proteins that bind to Coronavirus S protein. In certain embodiments, an epitope described herein or a sequence comprising an epitope described herein can be utilized to interfere with biological activity of Coronavirus S protein.

In some embodiments, antibodies or antigen-binding fragments thereof that bind to any of the epitopes are particularly useful. In some embodiments, an epitope provided herein, when bound by an antibody, modulates the biological activity of Coronavirus S protein.

In some embodiments, the domain(s)/region(s) containing residues that are in contact with or are buried by an antibody can be identified by mutating specific residues in Coronavirus S protein and determining whether the antibody can bind the mutated Coronavirus S protein protein. By making a number of individual mutations, residues that play a direct role in binding or that are in sufficiently close proximity to the antibody such that a mutation can affect binding between the antibody and antigen can be identified. From knowledge of these amino acids, the domain(s) or region(s) of the antigen that contain residues in contact with the antigen binding protein or covered by the antibody can be elucidated. Such a domain can include the binding epitope of an antigen binding protein.

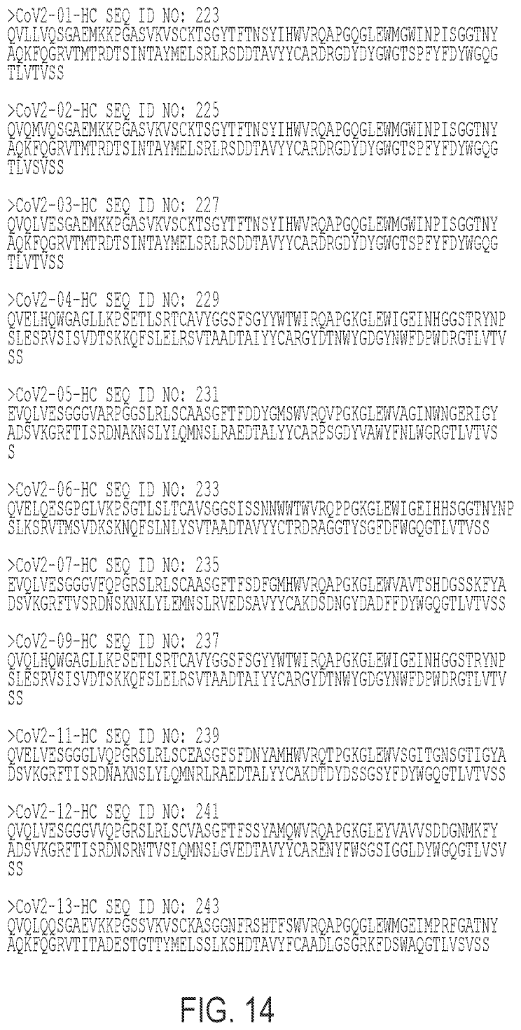

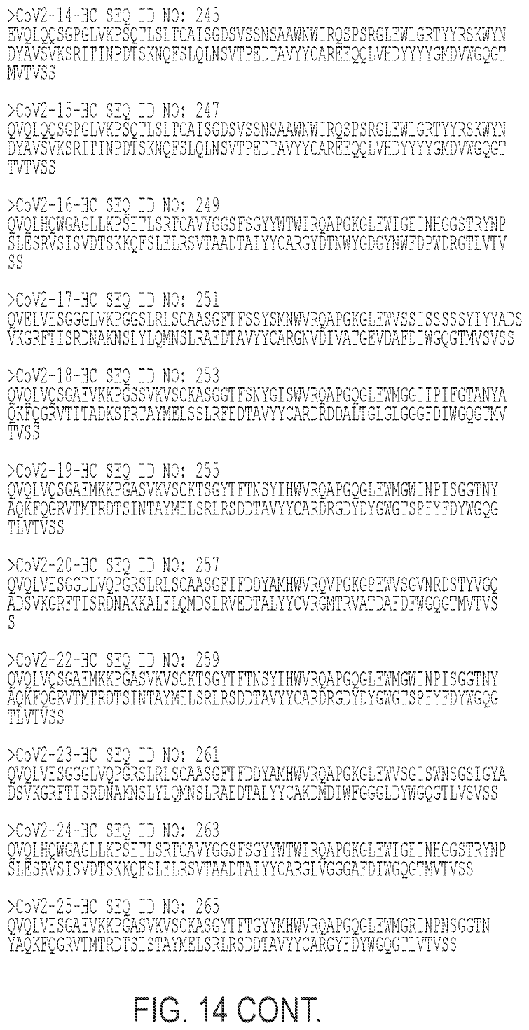

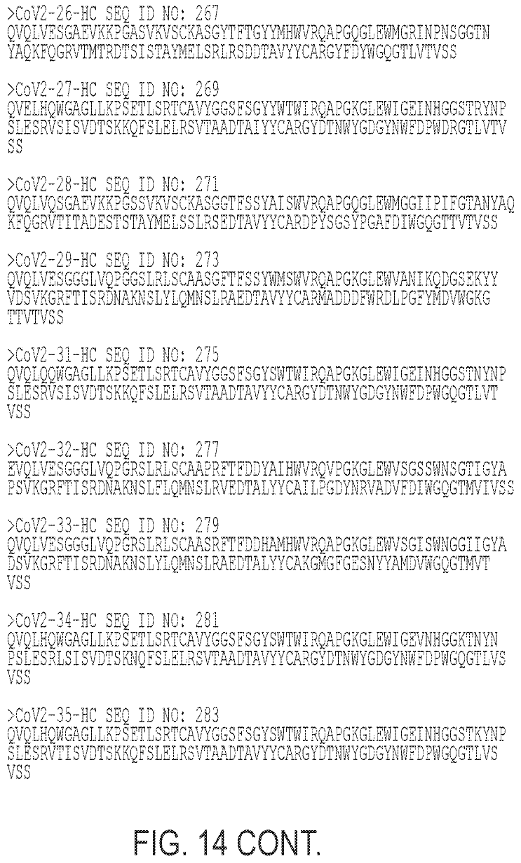

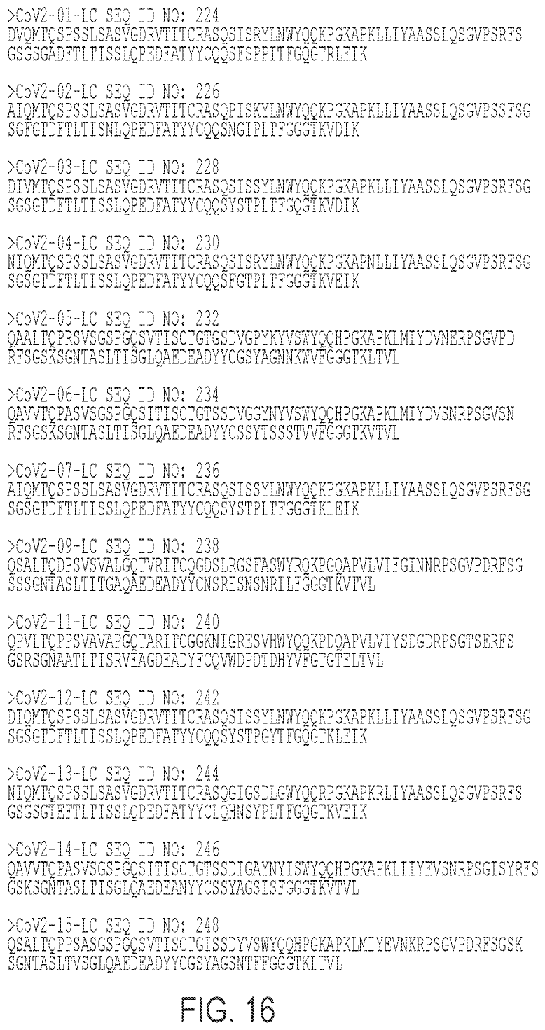

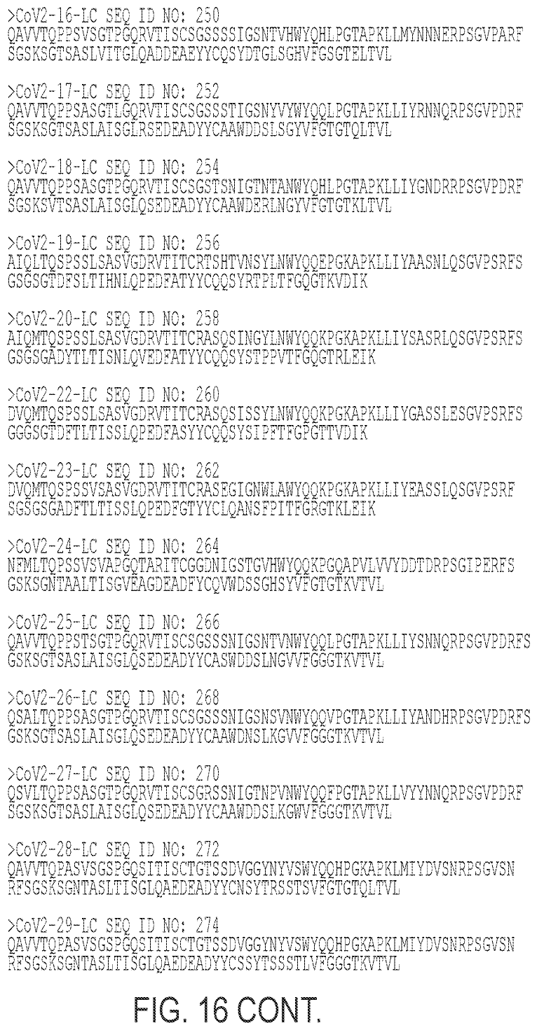

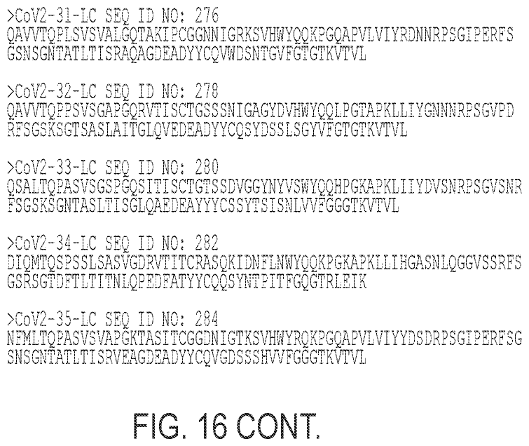

In another aspect, the present disclosure provides antigen-binding proteins that compete with one of the exemplified antibodies or antigen-binding fragment binding to the epitope described herein for specific binding to Coronavirus S protein. Such antigen binding proteins can also bind to the same epitope as one of the herein exemplified antibodies or the antigen-binding fragment, or an overlapping epitope. Antigen-binding proteins that compete with or bind to the same epitope as the exemplified antibodies are expected to show similar functional properties. The exemplified antibodies include those described above, including those with the heavy and light chain variable regions and CDRs included in FIGS. 14 and 16, Table A, and Table B.

C. Engineering of Antibody Sequences

In various embodiments, one can choose to engineer sequences of the identified antibodies for a variety of reasons, such as improved expression, improved cross-reactivity or diminished off-target binding. The following is a general discussion of relevant techniques for antibody engineering.

Hybridomas can be cultured, then cells lysed, and total RNA extracted. Random hexamers can be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization can be performed using antibodies collected from hybridoma supernatants and purified by FPLC, using Protein G columns. Recombinant full-length IgG antibodies can be generated by subcloning heavy and light chain Fv DNAs from the cloning vector into an IgG plasmid vector, transfected into 293 Freestyle cells or CHO cells, and antibodies collected a purified from the 293 or CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant methods well known to those of ordinary skill in the art. Such antibody derivatives are monovalent. In one embodiment, such fragments can be combined with one another, or with other antibody fragments or receptor ligands to form “chimeric” binding molecules. Significantly, such chimeric molecules can contain substituents capable of binding to different epitopes of the same molecule.

1. Antigen Binding Modifications

In related embodiments, the antibody is a derivative of the disclosed antibodies, e.g., an antibody comprising the CDR sequences identical to those in the disclosed antibodies (e.g., a chimeric, or CDR-grafted antibody). Alternatively, modifications can be made, such as introducing conservative changes into an antibody molecule. In making such changes, the hydropathic index of amino acids can be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids can be those resulting in hydrophilicity values within ±2 within ±1, or within ±0.5.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Modified antibodies can be made by any technique known to those of skill in the art, including expression through standard molecular biological techniques, or the chemical synthesis of polypeptides. Methods for recombinant expression are addressed elsewhere in this document.

2. Fc Region Modifications

The antibodies disclosed herein can also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antibodies disclosed herein can be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat. The antibodies disclosed herein also include antibodies with modified (or blocked) Fc regions to provide altered effector functions. See, e.g., U.S. Pat. No. 5,624,821; WO2003/086310; WO2005/120571; WO2006/0057702. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. This mutation has been reported to abolish the heterogeneity of inter-heavy chain disulfide bridges in the hinge region.

In one embodiment, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody. In another embodiment, the antibody is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022. In yet other embodiments, the Fc region is altered by replacing at least one amino acid residue with a different amino acid residue to alter the effector function(s) of the antibodies. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. No. 5,624,821 and

In another example, one or more amino acid residues within amino acid positions 231 and 239 are altered to thereby alter the ability of the antibody to fix complement. This approach is described further in PCT Publication WO 94/29351. In yet another example, the Fc region is modified to increase or decrease the ability of the antibodies to mediate antibody dependent cellular cytotoxicity (ADCC) and/or to increase or decrease the affinity of the antibodies for an Fcγ receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072. Moreover, the binding sites on human IgG1 for FcγR1, FcγRII, FcγRIII and FcRn have been mapped and variants with improved binding have been described. Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In one embodiment, the Fc region is modified to decrease the ability of the antibodies to mediate effector function and/or to increase anti-inflammatory properties by modifying residues 243 and 264. In one embodiment, the Fc region of the antibody is modified by changing the residues at positions 243 and 264 to alanine. In one embodiment, the Fc region is modified to decrease the ability of the antibody to mediate effector function and/or to increase anti-inflammatory properties by modifying residues 243, 264, 267 and 328. In still another embodiment, the antibody comprises a particular glycosylation pattern. For example, an aglycosylated antibody can be made (i.e., the antibody lacks glycosylation). The glycosylation pattern of an antibody can be altered to, for example, increase the affinity or avidity of the antibody for an antigen. Such modifications can be accomplished by, for example, altering one or more of the glycosylation sites within the antibody sequence. For example, one or more amino acid substitutions can be made that result removal of one or more of the variable region framework glycosylation sites to thereby eliminate glycosylation at that site. Such aglycosylation can increase the affinity or avidity of the antibody for antigen. See, e.g., U.S. Pat. Nos. 5,714,350 and 6,350,861.

An antibody can also be made in which the glycosylation pattern includes hypofucosylated or afucosylated glycans, such as a hypofucosylated antibodies or afucosylated antibodies have reduced amounts of fucosyl residues on the glycan. The antibodies can also include glycans having an increased amount of bisecting GlcNac structures. Such altered glycosylation patterns have been demonstrated to increase the ADCC ability of antibodies. Such modifications can be accomplished by, for example, expressing the antibodies in a host cell in which the glycosylation pathway was been genetically engineered to produce glycoproteins with particular glycosylation patterns. These cells have been described in the art and can be used as host cells in which to express recombinant antibodies of the disclosure to thereby produce an antibody with altered glycosylation. For example, the cell lines Ms704, Ms705, and Ms709 lack the fucosyltransferase gene, FUT8 (α(1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704. As another example, EP 1 176 195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1 176 195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell. Antibodies with a modified glycosylation profile can also be produced in chicken eggs, as described in PCT Publication WO 06/089231. Alternatively, antibodies with a modified glycosylation profile can be produced in plant cells, such as Lemna (U.S. Pat. No. 7,632,983). Methods for production of antibodies in a plant system are disclosed in the U.S. Pat. Nos. 6,998,267 and 7,388,081. PCT Publication WO 99/54342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies.

Alternatively, the fucose residues of the antibodies can be cleaved off using a fucosidase enzyme; e.g., the fucosidase α-L-fucosidase removes fucosyl residues from antibodies. Antibodies disclosed herein further include those produced in lower eukaryote host cells, in particular fungal host cells such as yeast and filamentous fungi have been genetically engineered to produce glycoproteins that have mammalian- or human-like glycosylation patterns. A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures.

In addition, since fungi such as yeast or filamentous fungi lack the ability to produce fucosylated glycoproteins, antibodies produced in such cells will lack fucose unless the cells are further modified to include the enzymatic pathway for producing fucosylated glycoproteins (See for example, PCT Publication WO2008112092). In particular embodiments, the antibodies disclosed herein further include those produced in lower eukaryotic host cells and which comprise fucosylated and nonfucosylated hybrid and complex N-glycans, including bisected and multiantennary species, including but not limited to N-glycans such as GlcNAc(1-4)Man3GlcNAc2; Gal(1-4)GlcNAc(1-4)Man3GlcNAc2; NANA(1-4)Gal(1-4)GlcNAc (1-4)Man3 GlcNAc2. In particular embodiments, the antibody compositions provided herein can comprise antibodies having at least one hybrid N-glycan selected from the group consisting of GlcNAcMan5GlcNAc2; GalGlcNAcMan5GlcNAc2; and NANAGalGlcNAcMan5GlcNAc2. In particular aspects, the hybrid N-glycan is the predominant N-glycan species in the composition. In further aspects, the hybrid N-glycan is a particular N-glycan species that comprises about 30%, 40%, 50%, 60%, 70%, 80%, 90%, 95%, 97%, 98%, 99%, or 100% of the hybrid N-glycans in the composition.

In particular embodiments, the antibody compositions provided herein comprise antibodies having at least one complex N-glycan selected from the group consisting of GlcNAcMan3GlcNAc2; GalGlcNAcMan3GlcNAc2; NANAGalGlcNAcMan3GlcNAc2; GlcNAc2Man3GlcNAc2; GalGlcNAc2Man3GlcNAc2; Gal2GlcNAc2Man3GlcNAc2; NANAGal2GlcNAc2Man3GlcNAc2; and NANA2Gal2GlcNAc2Man3GlcNAc2. In particular aspects, the complex N-glycan is the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In particular embodiments, the N-glycan is fusosylated. In general, the fucose is in an α1,3-linkage with the GlcNAc at the reducing end of the N-glycan, an α1,6-linkage with the GlcNAc at the reducing end of the N-glycan, an α1,2-linkage with the Gal at the non-reducing end of the N-glycan, an α1,3-linkage with the GlcNac at the non-reducing end of the N-glycan, or an α1,4-linkage with a GlcNAc at the non-reducing end of the N-glycan.

Therefore, in particular aspects of the above the glycoprotein compositions, the glycoform is in an α1,3-linkage or a1,6-linkage fucose to produce a glycoform selected from the group consisting of Man5GlcNAc2(Fuc), GlcNAcMan5GlcNAc2(Fuc), Man3GlcNAc2(Fuc), GlcNAcMan3GlcNAc2(Fuc), GlcNAc2Man3GlcNAc2(Fuc), GalGlcNAc2Man3GlcNAc2(Fuc), Gal2GlcNAc2Man3GlcNAc2(Fuc), NANAGal2GlcNAc2Man3GlcNAc2(Fuc), and NANA2Gal2GlcNAc2Man3GlcNAc2(Fuc); in an α1,3-linkage or a1,4-linkage fucose to produce a glycoform selected from the group consisting of GlcNAc(Fuc)Man5GlcNAc2, GlcNAc(Fuc)Man3GlcNAc2, GlcNAc2(Fuc1-2)Man3GlcNAc2, GalGlcNAc2(Fuc1-2)Man3GlcNAc2, Gal2GlcNAc2(Fuc1-2)Man3GlcNAc2, NANAGal2GlcNAc2(Fuc1-2)Man3GlcNAc2, and NANA2Gal2GlcNAc2(Fuc1-2)Man3GlcNAc2; or in an α1,2-linkage fucose to produce a glycoform selected from the group consisting of Gal(Fuc)GlcNAc2Man3GlcNAc2, Gal2(Fuc1-2)GlcNAc2Man3GlcNAc2, NANAGal2(Fuc1-2)GlcNAc2Man3GlcNAc2, and NANA2Gal2(Fuc1-2)GlcNAc2Man3 GlcNAc2.

In further aspects, the antibodies comprise high mannose N-glycans, including but not limited to, Man8GlcNAc2, Man7GlcNAc2, Man6GlcNAc2, Man5GlcNAc2, Man4GlcNAc2, or N-glycans that consist of the Man3GlcNAc2 N-glycan structure. In further aspects of the above, the complex N-glycans further include fucosylated and non-fucosylated bisected and multiantennary species. As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein.

D. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

The recombinant antibodies of the present disclosure can also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains can be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stablizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker can react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

In certain embodiments a cross-linker having reasonable stability in blood can be employed. Numerous types of disulfide-bond containing linkers are known that can be successfully employed to conjugate targeting and therapeutic/preventative agents. Linkers that contain a disulfide bond that is sterically hindered can give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest can be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about amino acids in length, contains at least one occurrence of a charged amino acid (e.g., arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Purification

In certain embodiments, the antibodies of the present disclosure can be purified. The term “purified,” as used herein, is intended to refer to a composition, isolatable from other components, wherein the protein is purified to any degree relative to its naturally-obtainable state. A purified protein therefore also refers to a protein, free from the environment in which it naturally occurs. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest can be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, the polypeptide can be expressed in a prokaryotic or eukaryotic expression system followed in some instances by extraction the protein using denaturing conditions. The polypeptide can be purified from other cellular components using an affinity column, which binds to a tagged portion of the polypeptide. As is generally known in the art, it is believed that the order of conducting the various purification steps can be changed, or that certain steps can be omitted, and still result in a suitable method for the preparation of a substantially purified protein or peptide.

Commonly, complete antibodies are fractionated utilizing agents (i.e., protein A) that bind the Fc portion of the antibody. Alternatively, antigens can be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products can vary.

### V. Treatment Methods

A. Formulation and Administration

The present disclosure provides pharmaceutical compositions comprising anti-Coronavirus S protein antibodies and antigens for generating the same. Such compositions comprise a prophylactically or therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, which can be in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Antibodies of the present disclosure, as described herein, can be formulated for parenteral administration, e.g., formulated for injection via the intradermal, intravenous, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

A. Antiviral-Cell Therapies

In another aspect, the present disclosure provides immune cells which express a chimeric antigen receptor (CAR). In some embodiment, The CAR comprises an antigen-binding fragment provided herein. In an embodiment, the CAR protein includes from the N-terminus to the C-terminus: a leader peptide, an anti-Coronavirus S protein heavy chain variable domain, a linker domain, an anti-Coronavirus S protein light chain variable domain, a human IgG1-CH2-CH3 domain, a spacer region, a CD28 transmembrane domain, a 4-1BB intracellular co-stimulatory signaling and a CD3 intracellular T cell signaling domain.

Also provided are methods for immunotherapy comprising administering an effective amount of the immune cells of the present disclosure. In one embodiments, a medical disease or disorder is treated by transfer of an immune cell population that elicits an immune response. In certain embodiments of the present disclosure, infection is treated by transfer of an immune cell population that elicits an immune response. Provided herein are methods for treating or delaying progression of viral disease in an individual comprising administering to the individual an effective amount an antigen-specific cell therapy.

The immune cells can be T cells (e.g., regulatory T cells, CD4+ T cells, CD8+ T cells, or gamma-delta T cells), NK cells, invariant NK cells, NKT cells, or macrophages. Also provided herein are methods of producing and engineering the immune cells as well as methods of using and administering the cells for adoptive cell therapy, in which case the cells can be autologous or allogeneic. Thus, the immune cells can be used as immunotherapy, such as to target virus-infected cells.

The immune cells can be isolated from subjects, particularly human subjects. The immune cells can be obtained from healthy human subjects, healthy volunteers, or healthy donors. The immune cells can be obtained from a subject of interest, such as a subject suspected of having a particular disease or condition, a subject suspected of having a predisposition to a particular disease or condition, or a subject who is undergoing therapy for a particular disease or condition Immune cells can be collected from any location in which they reside in the subject including, but not limited to, blood, cord blood, spleen, thymus, lymph nodes, and bone marrow. The isolated immune cells can be used directly, or they can be stored for a period of time, such as by freezing.

The immune cells can be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified can be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood can be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, e.g., a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor can be allogeneic, provided the cells obtained are subject-compatible in that they can be introduced into the subject. Allogeneic donor cells can, in some embodiments, be human-leukocyte-antigen (HLA)-compatible. To be rendered subject-compatible, allogeneic cells can be treated to reduce immunogenicity.

The immune cells can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a virus antigen. In particular embodiments, NK cells are engineered to express a TCR. The NK cells can be further engineered to express a CAR. Multiple CARs and/or TCRs, such as to different antigens, can be added to a single cell type, such as T cells or NK cells.

Suitable methods of modification are known in the art. See, for instance, Sambrook et al., supra; and Ausubel et al., CURRENT PROTOCOLS IN MOLECULAR BIOLOGY, Greene Publishing Associates and John Wiley & Sons, N Y, 1994. For example, the cells can be transduced to express a T cell receptor (TCR) having antigenic specificity for a viral antigen using transduction techniques described in Heemskerk et al. (2008) and Johnson et al. (2009).

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

C. Combination Therapies

In some embodiments, combination treatments are provided using antibodies of the present disclosure in conjunction with additional anti-viral therapies. These therapies would be provided in a combined amount effective to achieve a reduction in one or more disease parameter. This process can involve contacting the cells/subjects with the both agents/therapies at the same time, e.g., using a single composition or pharmacological formulation that includes both agents, or by contacting the cell/subject with two distinct compositions or formulations, at the same time, wherein one composition includes the antibody and the other includes the other agent.

Alternatively, the antibody can precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, the time period for treatment can be extended significantly; however, where several 10 days (2, 3, 4, 5, 6 or 7) to several weeks (1, 2, 3, 4, 5, 6, 7 or 8) lapse between the respective administrations.

It also is conceivable that more than one administration of either the anti-CH3L1 antibody or the other therapy will be desired. Various combinations can be employed, where the antibody is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. To kill cells, inhibit cell growth, inhibit metastasis, inhibit angiogenesis or otherwise reverse or reduce the malignant phenotype of tumor cells, using the methods and compositions of the present disclosure, one can contact a target cell or site with an antibody and at least one other therapy. These therapies would be provided in a combined amount effective to kill or inhibit proliferation of virus. This process can involve contacting the cells/site/subject with the agents/therapies at the same time.

Particular agents contemplated for combination therapy with antibodies of the present disclosure include chemotherapy and hematopoietic stem cell transplantation. Chemotherapy can include cytarabine (ara-C) and an anthracycline (most often daunorubicin), high-dose cytarabine alone, all-trans-retinoic acid (ATRA) in addition to induction chemotherapy, usually an anthracycline, histamine dihydrochloride (Ceplene) and interleukin 2 (Proleukin) after the completion of consolidation therapy, gemtuzumab ozogamicin (Mylotarg) for patients aged more than 60 years with relapsed AML who are not candidates for high-dose chemotherapy, clofarabine, as well as targeted therapies, such as kinase inhibitors, farnesyl transferase inhibitors, decitabine, and inhibitors of MDR1 (multidrug-resistance protein), or arsenic trioxide or relapsed acute promyelocytic leukemia (APL).

In certain embodiments, the agents for combination therapy are one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICA®, an acalabrutinib, CALQUENCE®, a zanubrutinib, a PD-1 antibody, a PD-L1 antibody, a CTLA-4 antibody, a LAGS antibody, an ICOS antibody, a TIGIT antibody, a TIM3 antibody, a CD40 antibody, a 4-1BB antibody, a CD47 antibody, a SIRPloc antibody or fusions protein, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

C. Hepatotoxicity Combination Therapies

In certain embodiments, combination treatments are provided using antibodies of the present disclosure in conjunction with additional hepatotoxicity therapies. These therapies would be provided in a combined amount effective to achieve a reduction in one or more disease parameter. This process can involve contacting the cells/subjects with both agents/therapies at the same time, e.g., using a single composition or pharmacological formulation that includes both agents, or by contacting the cell/subject with two distinct compositions or formulations, at the same time, wherein one composition includes the antibody and the other includes the other agent.

Alternatively, the antibody can precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, the time period for treatment can be extended significantly; however, where several 10 days (2, 3, 4, 5, 6 or 7) to several weeks (1, 2, 3, 4, 5, 6, 7 or 8) lapse between the respective administrations.

It also is conceivable that more than one administration of either the anti-CH3L1 antibody or the other therapy will be desired. Various combinations can be employed, where the antibody is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. This process can involve contacting the cells/site/subject with the agents/therapies at the same time or at different times. The other therapy can be supportive care, including pain medication and fluids, and in some instances an anti-toxin.

### VI. Antibody Conjugates

Antibodies of the present disclosure can be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety can be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which can be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

Antibody conjugates can also be used as as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III). In certain embodiments the paramagnetic ion is gadolinium. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I can be used in certain embodiments, and technicium^{99m} and/or indium¹¹¹ can also be effective due to their low energy and suitability for long range detection. Radioactively labeled monoclonal antibodies of the present disclosure can be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure can be labeled with technetium^{99m} by ligand exchange process, for example, by reducing pertechnate with stannous solution, chelating the reduced technetium onto a Sephadex column and applying the antibody to this column. Alternatively, direct labeling techniques can be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. In certain embodiments the secondary binding ligands can be biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this can, in some situations, be disadvantageous since it results in loss of antigen binding by the antibody conjugate.

Molecules containing azido groups can also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and can be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies can also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### VII. Immunodetection Methods

In still further embodiments, the present disclosure concerns immunodetection methods for binding, purifying, removing, quantifying and otherwise generally detecting Coronavirus S protein-related cancers. While such methods can be applied in a traditional sense, another use will be in quality control and monitoring of vaccine stocks, where antibodies according to the present disclosure can be used to assess the amount or integrity (i.e., long term stability) of antigens. Alternatively, the methods can be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of Coronavirus S protein also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing Coronavirus S protein, and contacting the sample with a first antibody in accordance with the present disclosure under conditions effective to allow the formation of immunocomplexes.

These methods include methods for detecting or purifying Coronavirus S protein or Coronavirus S protein from a sample. The antibody can be linked to a solid support, such as in the form of a column matrix, and the sample suspected of containing the Coronavirus S protein-related cancer cells will be applied to the immobilized antibody. The unwanted components will be washed from the column, leaving the Coronavirus S protein-expressing cells immunocomplexed to the immobilized antibody, which is then collected by removing the organism or antigen from the column.

The immunobinding methods also include methods for detecting and quantifying the amount of Coronavirus S protein or related components in a sample and the detection and quantification of any immune complexes formed during the binding process. Here, one would obtain a sample suspected of containing Coronavirus S protein and contact the sample with an antibody that binds Coronavirus S protein or components thereof, followed by detecting and quantifying the amount of immune complexes formed under the specific conditions. In terms of antigen detection, the biological sample analyzed can be any sample that is suspected of containing Coronavirus S protein, such as a tissue section or specimen, a homogenized tissue extract, a biological fluid (e.g., a nasal swab), including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to Coronavirus S protein. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and can be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, in certain embodiments a secondary binding ligand such as a second antibody and/or a biotin/avidin ligand binding arrangement can be used, as is known in the art.

The antibody employed in the detection can itself be linked to a detectable label, wherein one would then simply detect this label, thereby allowing the amount of the primary immune complexes in the composition to be determined. Alternatively, the first antibody that becomes bound within the primary immune complexes can be detected by a second binding ligand that has binding affinity for the antibody. In these cases, the second binding ligand can be linked to a detectable label. The second binding ligand is itself often an antibody, termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system can provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

1. ELISAs

Immunoassays, in their most simple and direct sense, are binding assays. Certain immunoassays are the various types of enzyme linked immunosorbent assays (ELISAs) and radioimmunoassays (RIA) known in the art Immunohistochemical detection using tissue sections is also particularly useful. However, it will be readily appreciated that detection is not limited to such techniques, and western blotting, dot blotting, FACS analyses, and the like can also be used.

In one exemplary ELISA, the antibodies of the disclosure are immobilized onto a selected surface exhibiting protein affinity, such as a well in a polystyrene microtiter plate. Then, a test composition suspected of containing the Coronavirus S protein is added to the wells. After binding and washing to remove non-specifically bound immune complexes, the bound antigen is detected. Detection can be achieved by the addition of another anti-Coronavirus S protein antibody that is linked to a detectable label. This type of ELISA is a simple “sandwich ELISA.” Detection can also be achieved by the addition of a second anti-Coronavirus S protein antibody, followed by the addition of a third antibody that has binding affinity for the second antibody, with the third antibody being linked to a detectable label.

In another exemplary ELISA, the samples suspected of containing the Coronavirus S protein (e.g., potentially infected cells) are immobilized onto the well surface and then contacted with the anti-Coronavirus S protein antibodies of the disclosure. After binding and washing to remove non-specifically bound immune complexes, the bound anti-Coronavirus S protein antibodies are detected. Where the initial anti-Coronavirus S protein antibodies are linked to a detectable label, the immune complexes can be detected directly. Again, the immune complexes can be detected using a second antibody that has binding affinity for the first anti-Coronavirus S protein antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection methods rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions can include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures on the order of, e.g., 25° C. to 27° C., or can be overnight at about 4° C. or so.

Following all incubation steps in an ELISA, the contacted surface is washed so as to remove non-complexed material. In certain embodiments the washing procedure can include washing with a solution such as PBS/Tween, or borate buffer. Following the formation of specific immune complexes between the test sample and the originally bound material, and subsequent washing, the occurrence of even minute amounts of immune complexes can be determined.

In order to detect binding, the second or third antibody can have an associated label to allow detection. In certain embodiments this can be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples can be taken from whole tissue or from cell culture. In most cases, solid tissues are first broken down mechanically using a blender (for larger sample volumes), using a homogenizer (smaller volumes), or by sonication. Cells can also be broken open by one of the above mechanical methods. Assorted detergents, salts, and buffers can be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins can be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

3. Immunohistochemistry

The antibodies of the present disclosure can also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections can be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples can be used for serial section cuttings.

Permanent sections can be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples can be substituted.

4. Immunodetection Kits

In still further embodiments, the present disclosure concerns immunodetection kits for use with the immunodetection methods described above. As the antibodies can be used to detect Coronavirus S protein, the antibodies can be included in the kit. The immunodetection kits will thus comprise, in a suitable container, a first antibody that binds to an Coronavirus S protein, and optionally an immunodetection reagent.

In certain embodiments, the antibody can be pre-bound to a solid support, such as a column matrix and/or well of a microtitre plate. The immunodetection reagents of the kit can take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels can be employed in connection with the present disclosure.

The kits can further comprise a suitably aliquoted composition of Coronavirus S protein, whether labeled or unlabeled, to be used to prepare a standard curve for a detection assay. The kits can contain antibody-label conjugates either in fully conjugated form, in the form of intermediates, or as separate moieties to be conjugated by the user of the kit. The components of the kits can be packaged either in aqueous media or in lyophilized form.

The container of the kits will generally include at least one vial, test tube, flask, bottle, syringe or other container, into which the antibody can be placed, or suitably aliquoted. The kits of the present disclosure will also typically include container for the antibody, antigen, and any other reagent containers in close confinement for commercial sale. Such containers can include injection or blow-molded plastic containers into which the desired vials are retained.

5. Flow Cytometry and FACS

The antibodies of the present disclosure can also be used in flow cytometry or FACS. Flow cytometry is a laser- or impedance-based technology employed in many detection assays, including cell counting, cell sorting, biomarker detection and protein engineering. The technology suspends cells in a stream of fluid and passing them through an electronic detection apparatus, which allows simultaneous multiparametric analysis of the physical and chemical characteristics of up to thousands of particles per second. Flow cytometry is routinely used in the diagnosis disorders, especially blood cancers, but has many other applications in basic research, clinical practice and clinical trials.

Fluorescence-activated cell sorting (FACS) is a specialized type of cytometry. It provides a method for sorting a heterogenous mixture of biological cells into two or more containers, one cell at a time, based on the specific light scattering and fluorescent characteristics of each cell. In general, the technology involves a cell suspension entrained in the center of a narrow, rapidly flowing stream of liquid. The flow is arranged so that there is a large separation between cells relative to their diameter. A vibrating mechanism causes the stream of cells to break into individual droplets. Just before the stream breaks into droplets, the flow passes through a fluorescence measuring station where the fluorescence of each cell is measured. An electrical charging ring is placed just at the point where the stream breaks into droplets. A charge is placed on the ring based immediately prior to fluorescence intensity being measured, and the opposite charge is trapped on the droplet as it breaks form the stream. The charged droplets then fall through an electrostatic deflection system that diverts droplets into containers based upon their charge.

In certain embodiments, to be used in flow cytometry or FACS, the antibodies of the present disclosure are labeled with fluorophores and then allowed to bind to the cells of interest, which are analyzed in a flow cytometer or sorted by a FACS machine.

### VIII. Exemplary Embodiments

The present disclosure includes the following exemplary embodiments:

1. An isolate monoclonal antibody or an antigen-binding fragment thereof comprising a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 31, SEQ ID NO: 32, SEQ ID NO: 33, SEQ ID NO: 34, SEQ ID NO: 35, and SEQ ID NO: 36; SEQ ID NO: 43, SEQ ID NO: 44, SEQ ID NO: 45, SEQ ID NO: 46, SEQ ID NO: 47, and SEQ ID NO: 48; SEQ ID NO: 55, SEQ ID NO: 56, SEQ ID NO: 57, SEQ ID NO: 58, SEQ ID NO: 59, and SEQ ID NO: 60; SEQ ID NO: 67, SEQ ID NO: 68, SEQ ID NO: 69, SEQ ID NO: 70, SEQ ID NO: 71, and SEQ ID NO: 72; SEQ ID NO: 79, SEQ ID NO: 80, SEQ ID NO: 81, SEQ ID NO: 82, SEQ ID NO: 83, and SEQ ID NO: 84; SEQ ID NO: 133, SEQ ID NO: 134, SEQ ID NO: 135, SEQ ID NO: 136, SEQ ID NO: 137, and SEQ ID NO: 138; SEQ ID NO: 187, SEQ ID NO: 188, SEQ ID NO: 189, SEQ ID NO: 190, SEQ ID NO: 191, and SEQ ID NO: 192; SEQ ID NO: 193, SEQ ID NO: 194, SEQ ID NO: 195, SEQ ID NO: 196, SEQ ID NO: 197, and SEQ ID NO: 198; SEQ ID NO: 199, SEQ ID NO: 200, SEQ ID NO: 201, SEQ ID NO: 202, SEQ ID NO: 203, and SEQ ID NO: 204; SEQ ID NO: 205, SEQ ID NO: 206, SEQ ID NO: 207, SEQ ID NO: 208, SEQ ID NO: 209, and SEQ ID NO: 210; SEQ ID NO: 211, SEQ ID NO: 212, SEQ ID NO: 213, SEQ ID NO: 214, SEQ ID NO: 215, and SEQ ID NO: 216; or SEQ ID NO: 217, SEQ ID NO: 218, SEQ ID NO: 219, SEQ ID NO: 220, SEQ ID NO: 221, and SEQ ID NO: 222.

2. The isolated monoclonal antibody or an antigen-binding fragment thereof of embodiment 1, wherein the HCDR1, HCDR2, HCDR3, LCDR1, LCDR2, and LCDR3, comprise, respectively, the amino acid sequences SEQ ID NO: 67, SEQ ID NO: 68, SEQ ID NO: 69, SEQ ID NO: 70, SEQ ID NO: 71, and SEQ ID NO: 72.

3. The isolated monoclonal antibody or an antigen-binding fragment thereof of embodiment 1, wherein the HCDR1, HCDR2, HCDR3, LCDR1, LCDR2, and LCDR3, comprise, respectively, the amino acid sequences SEQ ID NO: 205, SEQ ID NO: 206, SEQ ID NO: 207, SEQ ID NO: 208, SEQ ID NO: 209, and SEQ ID NO: 210.

4. The isolated monoclonal antibody or an antigen-binding fragment thereof of embodiment 1, wherein the VH and VL comprise amino acid sequences having at least 80%, 90%, or 95% identity to the amino acid sequences SEQ ID NO: 233 and SEQ ID NO: 234, SEQ ID NO: 237 and SEQ ID NO: 238, SEQ ID NO: 241 and SEQ ID NO: 242, SEQ ID NO: 245 and SEQ ID NO: 246, SEQ ID NO: 249 and SEQ ID NO: 250, or SEQ ID NO: 267 and SEQ ID NO: 268, respectively.

5. The isolated monoclonal antibody or an antigen-binding fragment thereof of embodiment 1, wherein the VH and VL comprise amino acid sequences having at least 80%, 90%, or 95% identity to the amino acid sequences SEQ ID NO: 245 and SEQ ID NO: 246, respectively.

6. The isolated monoclonal antibody or an antigen-binding fragment thereof of embodiment 1, wherein the VH and VL comprise the amino acid sequences SEQ ID NO: 233 and SEQ ID NO: 234, SEQ ID NO: 237 and SEQ ID NO: 238, SEQ ID NO: 241 and SEQ ID NO: 242, SEQ ID NO: 245 and SEQ ID NO: 246, SEQ ID NO: 249 and SEQ ID NO: 250, or SEQ ID NO: 267 and SEQ ID NO: 268, respectively.

7. The isolated monoclonal antibody or an antigen-binding fragment thereof of embodiment 1, wherein the VH and VL comprise the amino acid sequences SEQ ID NO: 245 and SEQ ID NO: 246, respectively.

8. An isolated monoclonal antibody or an antigen-binding fragment thereof comprising cloned paired heavy and light chain CDRs from Table A or Table B.

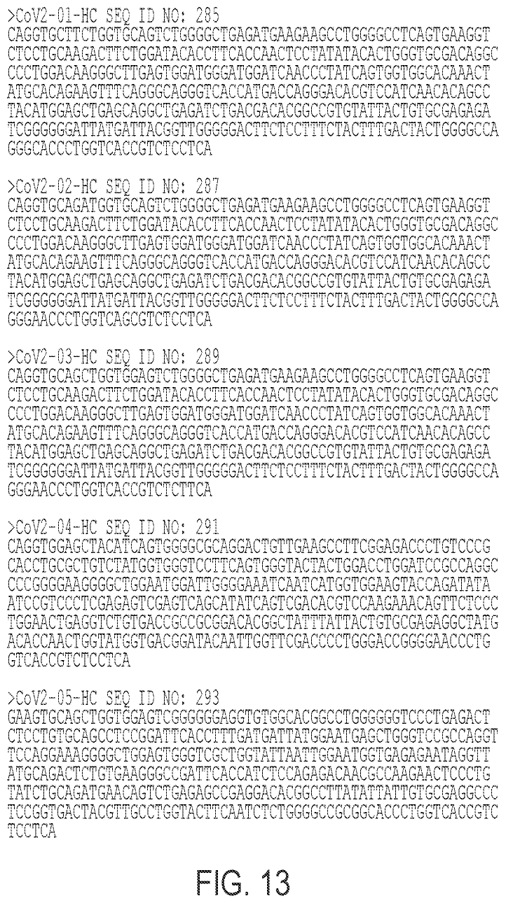

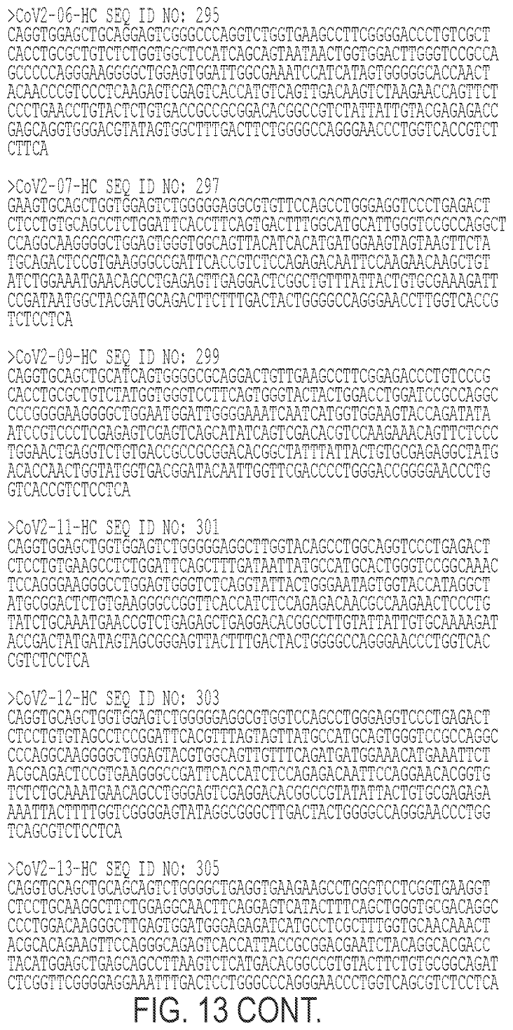

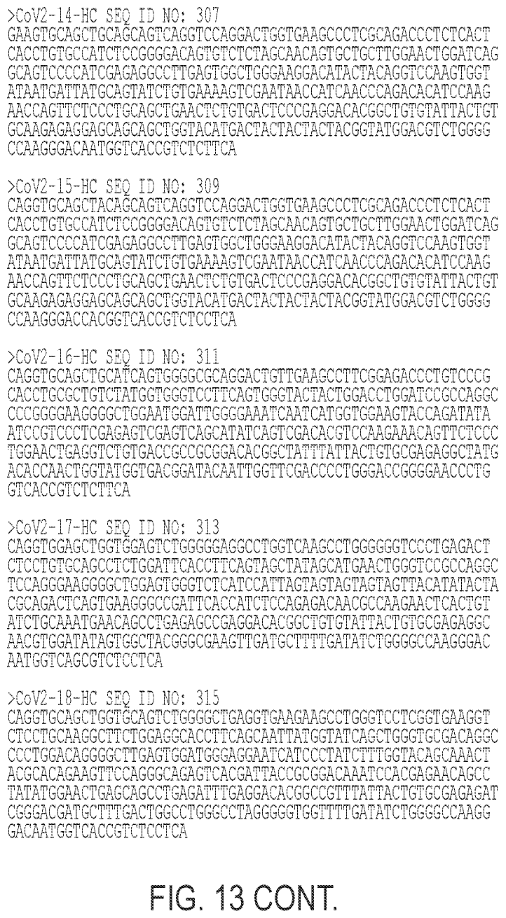

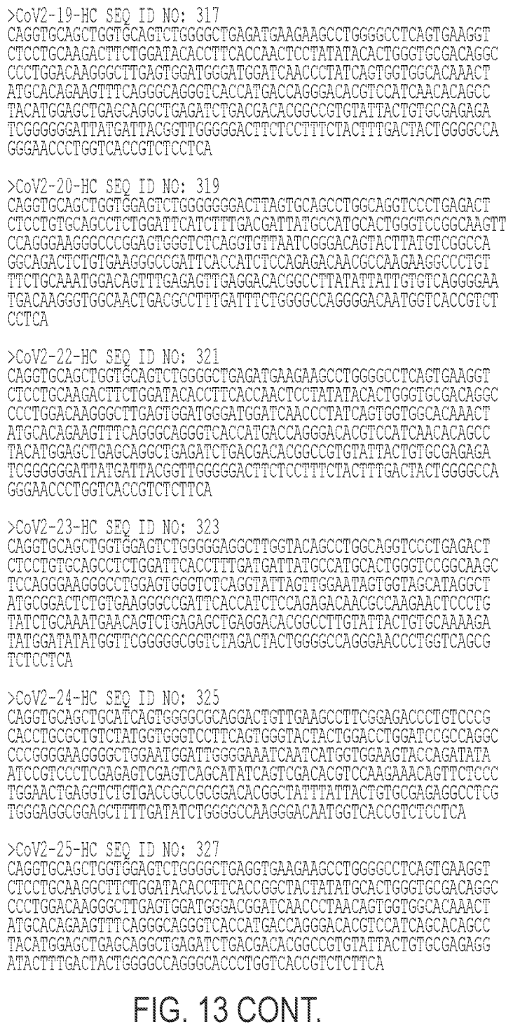

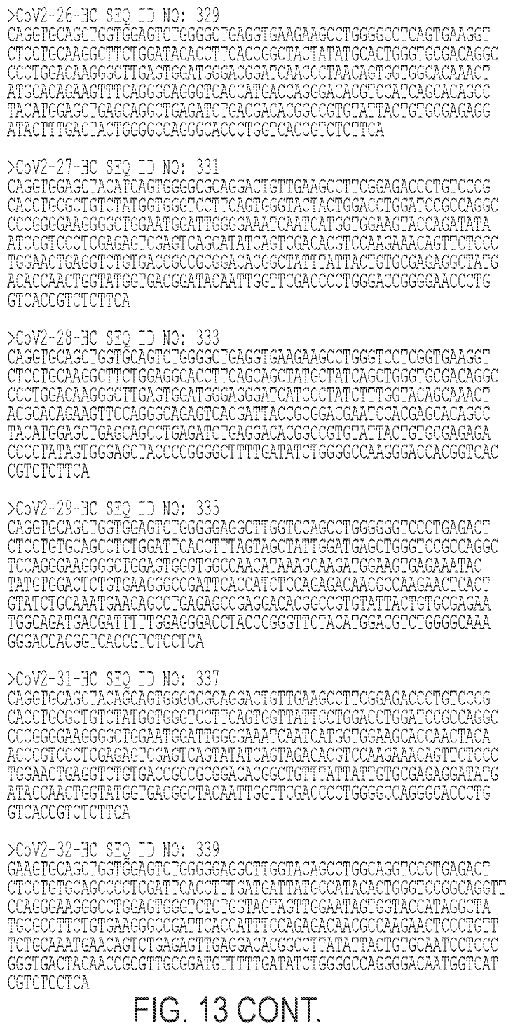

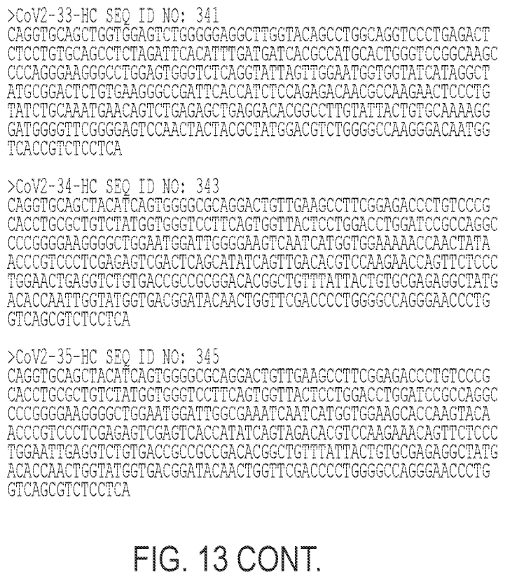

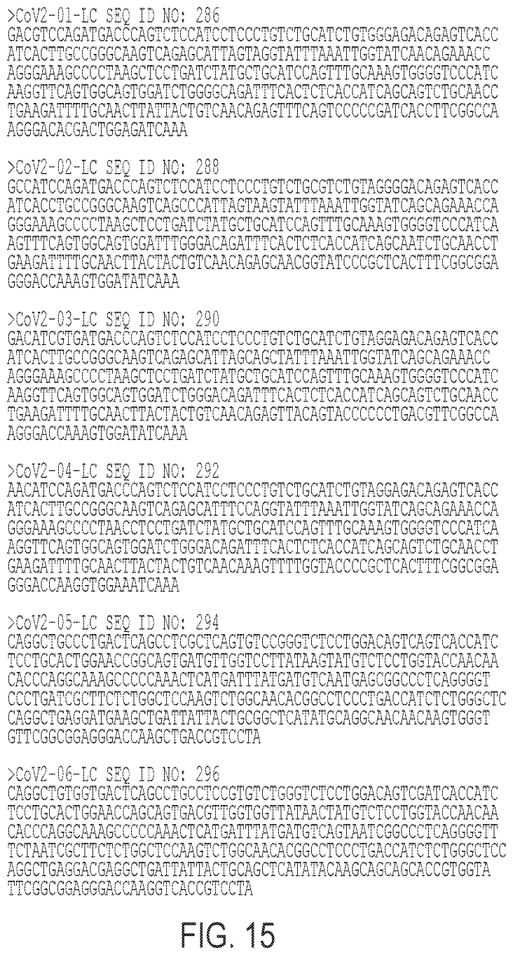

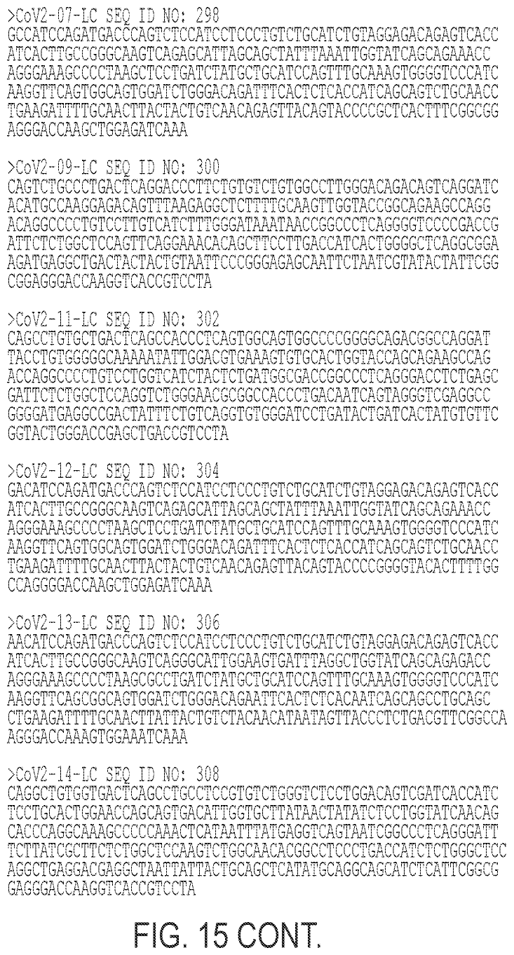

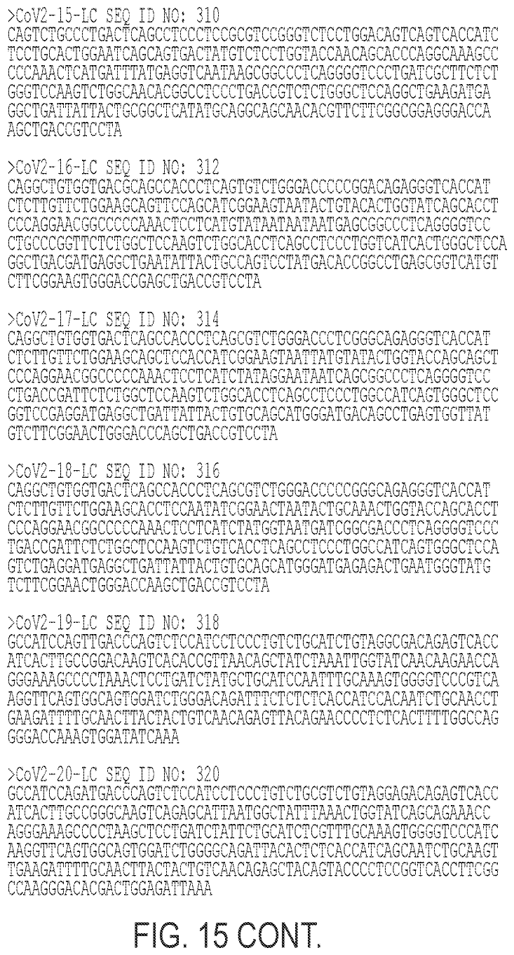

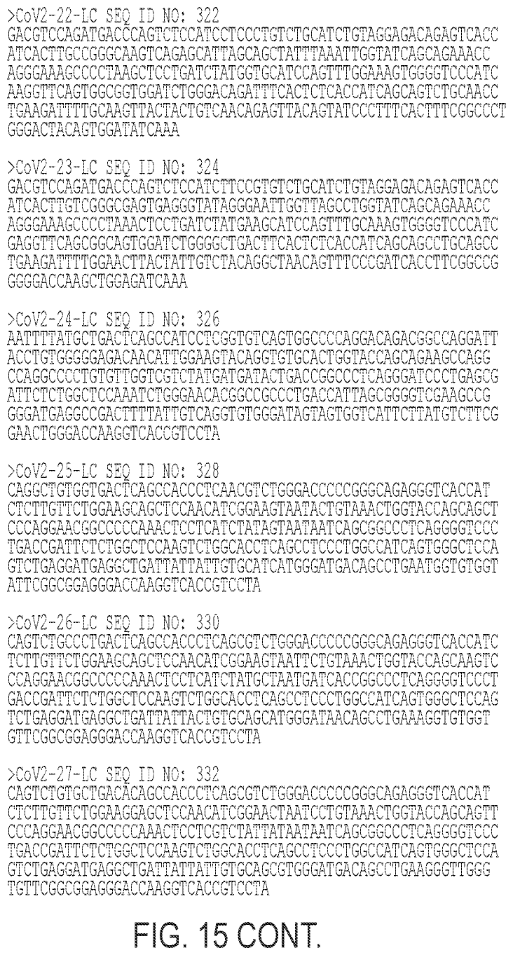

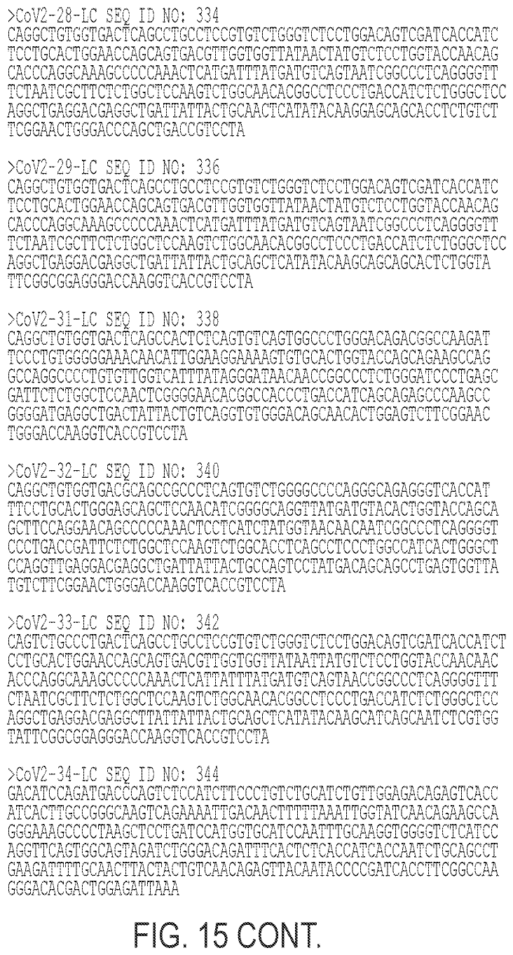

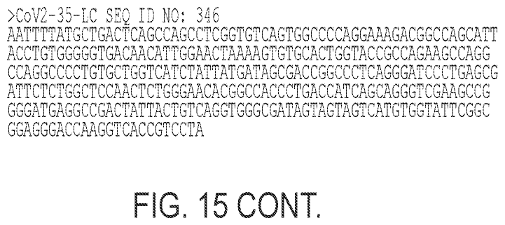

9. The isolated monoclonal antibody or an antigen-binding fragment thereof of embodiment 8, wherein antibody or fragment thereof is encoded by clone-paired heavy and light chain sequences from FIGS. 13 and 15, respectively.

10. The isolated monoclonal antibody or an antigen-binding fragment thereof of embodiment 8, wherein antibody or fragment thereof is encoded by heavy and light chain variable sequences having at least 70%, 80%, 90% or 95% identity to clone-paired sequences from FIGS. 13 and 15, respectively.

11. The isolated monoclonal antibody or an antigen-binding fragment thereof of embodiment 8, wherein antibody or fragment thereof comprises clone-paired heavy and light chain variable region sequences from FIGS. 14 and 16, respectively.

12. The isolated monoclonal antibody or an antigen-binding fragment thereof of embodiment 8, wherein antibody or fragment thereof comprises heavy and light chain variable sequences having at least 70%, 80%, 90% or 95% identity to clone-paired sequences from FIGS. 14 and 16, respectively.

13. An isolated monoclonal antibody or an antigen-binding fragment thereof wherein said antibody binds to the RGB domain (319-541) of SAR-CoV-2 and exhibits SAR-CoV-2 neutralizing activity.

14. The isolated monoclonal antibody or antigen binding fragment thereof of embodiment 13, wherein the antibody exhibits a neutralization activity (effective concentration 50; EC50) of less than 20, 10 or 5 (μg/ml).

15. The isolated monoclonal antibody or antigen binding fragment thereof of embodiment 14, wherein the antibody exhibits a neutralization activity of EC50 of about 0.1 to 20 (μg/ml).

16. The isolated monoclonal antibody or antigen binding fragment thereof of any one of embodiments 13-15, wherein the antibody also binds to the SARS-CoV S protein.

17. The isolated monoclonal antibody or antigen binding fragment thereof of embodiment 16, wherein the antibody binds to the RBD domain (306-527) of the SARS-CoV S protein.

18. The isolated monoclonal antibody or antigen binding fragment thereof of embodiment 16 or 17, wherein the antibody exhibits neutralizing activity of SARS-CoV.

19. The isolated monoclonal antibody or an antigen binding fragment thereof of any one of embodiments 1-18, wherein the isolated monoclonal antibody is a murine, a rodent, or a rabbit antibody.

20. The isolated monoclonal antibody or an antigen binding fragment thereof of any one of embodiments 1-18, wherein the isolated monoclonal antibody is a humanized, or human antibody.

21. The isolated monoclonal antibody or an antigen-binding fragment thereof of any one of embodiments 1-18, wherein the antigen-binding fragment is a recombinant ScFv (single chain fragment variable) antibody, Fab fragment, F(ab′)2 fragment, or Fv fragment.

22. The isolated monoclonal antibody or an antigen binding fragment thereof of any one of embodiments 1-18, wherein the isolated monoclonal antibody is a bispecific antibody or a chimeric antibody.

23. The isolated monoclonal antibody or antigen binding fragment thereof of any one of embodiments 1-18, wherein said antibody is an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

24. The isolated monoclonal antibody or antigen binding fragment thereof of any one of embodiments 1-23, wherein the antibody binds to a coronavirus spike (S) protein.

25. The isolated monoclonal antibody or antigen binding fragment thereof of embodiment 24, wherein the antibody binds to the SARS-CoV-2 S protein.

26. The isolated monoclonal antibody or antigen binding fragment thereof of embodiment 25, wherein the antibody binds to the RBD domain (319-541) of the SARS-CoV-2 S protein.

27. The isolated monoclonal antibody or antigen binding fragment thereof of any one of embodiments 24-26, wherein the antibody binds to the SARS-CoV S protein.

28. The isolated monoclonal antibody or antigen binding fragment thereof of embodiment 27, wherein the antibody binds to the RBD domain (306-527) of the SARS-CoV S protein.

29. The isolated monoclonal antibody or antigen binding fragment thereof of embodiment 24, wherein the antibody binds to the SARS-CoV S protein and the SARS-CoV-2 S protein.

30. The isolated monoclonal antibody or antigen binding fragment thereof of any one of embodiments 1-24, wherein the antibody is a virus neutralizing antibody.

31. The isolated monoclonal antibody or antigen binding fragment thereof of embodiment 30, wherein the antibody exhibits a neutralization activity (effective concentration 50; EC50) of less than 20, 10 or 5 (μg/ml).

32. The isolated monoclonal antibody or antigen binding fragment thereof of embodiment 31, wherein the antibody exhibits a neutralization activity EC50 of about to 20 (μg/ml).

33. The isolated monoclonal antibody or antigen binding fragment thereof of any one of embodiments 30-32, wherein the antibody is a SARS-CoV neutralizing antibody.

34. The isolated monoclonal antibody or antigen binding fragment thereof of any one of embodiment 34, wherein the antibody is a SARS-CoV-2 neutralizing antibody.

35. The isolated monoclonal antibody or antigen binding fragment thereof of any one of embodiments 30-32, wherein the antibody is a SARS-CoV and SARS-CoV-2 neutralizing antibody.

36. An isolated monoclonal antibody or an antigen binding fragment thereof, which competes for the same epitope with the isolated monoclonal antibody or an antigen-binding fragment thereof according to any of embodiments 1-12.

37. A pharmaceutical composition comprising the isolated monoclonal antibody or an antigen-binding fragment thereof according to any of embodiments 1-36, and a pharmaceutically acceptable carrier.

38. An isolated nucleic acid that encodes the isolated monoclonal antibody according to any of embodiments 1-36.

39. A vector comprising the isolated nucleic acid of embodiment 38.

40. A host cell comprising the vector of embodiment 39.

41. The host cell of embodiment 40, wherein the host cell is a mammalian cell.

42. The host cell of embodiment 40, wherein the host cell is a CHO cell.

43. A hybridoma encoding or producing the isolated monoclonal antibody according to any of embodiments 1-36.

44. A process of producing an antibody, comprising culturing the host cell of any one of embodiments 40-42 under conditions suitable for expressing the antibody, and recovering the antibody.

45. A chimeric antigen receptor (CAR) protein comprising an antigen-binding fragment according to any of embodiments 1-36.

46. An isolated nucleic acid that encodes a CAR protein of embodiment 45.

47. A vector comprising the isolated nucleic acid of embodiment 46.

48. An engineered cell comprising the isolated nucleic acid of embodiment 47.

49. The engineered cell of embodiment 48, wherein the cell is a T cell, NK cell, or macrophage.

50. A method of treating or ameliorating a Coronavirus infection in a subject, the method comprising administering to the subject a therapeutically effective amount of the antibody or an antigen-binding fragment thereof according to any of embodiments 1-36 or the engineered cell of embodiments 48 or 49.

51. The method of embodiment 50, wherein the method reduces viral replication in the subject.

52. The method of embodiment 50 or 51, wherein the method reduces inflammation in the lungs of a subject.

53. The method of any one of embodiments 50-52, wherein the subject is infected with SARS-CoV.

54. The method of any one of embodiments 50-52, wherein the subject is infected with SARS-CoV-2.

55. The method of any one of embodiments 50-54, wherein the subject has pneumonia.

56. The method of any one of embodiments 50-55, wherein the subject is on a respirator or oxygen supplementation.

57. The method of any one of embodiments 50-56, wherein the antibody or an antigen-binding fragment thereof is administered intravenously, intra-arterially, subcutaneously or via inhalation.

58. The method of any one of embodiments 50-57, further comprising administering to the subject a second anti-viral therapy.

59. A method of detecting coronavirus, coronavirus S protein and/or coronavirus-infected cells in a sample or subject comprising: (a) contacting a subject or a sample from the subject with the antibody or an antigen-binding fragment thereof according to any of embodiments 1-36; and (b) detecting binding of said antibody to a cancer cell or cancer stem cell in said subject or sample.

60. The method of embodiment 59, wherein the sample is a body fluid or biopsy.

61. The method of embodiment 59, wherein the sample is blood, bone marrow, sputum, tears, saliva, mucous, serum, urine, feces or a nasal swab.

62. The method of any one of embodiments 59-61, wherein detection comprises immunohistochemistry, flow cytometry, FACS, ELISA, RIA or Western blot.

63. The method of any one of embodiments 59-62, further comprising performing steps (a) and (b) a second time and determining a change in detection levels as compared to the first time.

64. The method of any one of embodiments 59-63, wherein said isolated monoclonal antibody or an antigen binding fragment thereof further comprises a label.

65. The method of embodiment 64, wherein said label is a peptide tag, an enzyme, a magnetic particle, a chromophore, a fluorescent molecule, a chemo-luminescent molecule, or a dye.

66. The method according to any of embodiments 50-65, wherein said isolated monoclonal antibody or an antigen binding fragment thereof is conjugated to a liposome or nanoparticle.

### IX. Examples

The following examples are included to demonstrate certain embodiments of the disclosure. It should be appreciated by those of skill in the art that the techniques disclosed in the examples which follow represent techniques discovered by the inventor to function well in the practice of the embodiments of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Development of SARS-CoV-2 S-Binding Antibodies

Studies detailed herein utilized phage panning to select antibodies that inhibit the binding of the RBD of SARS-CoV-2 and which would be expected to interfere with virus ability to bind ACE2 and have neutralizing activity (FIGS. 1A-1F).

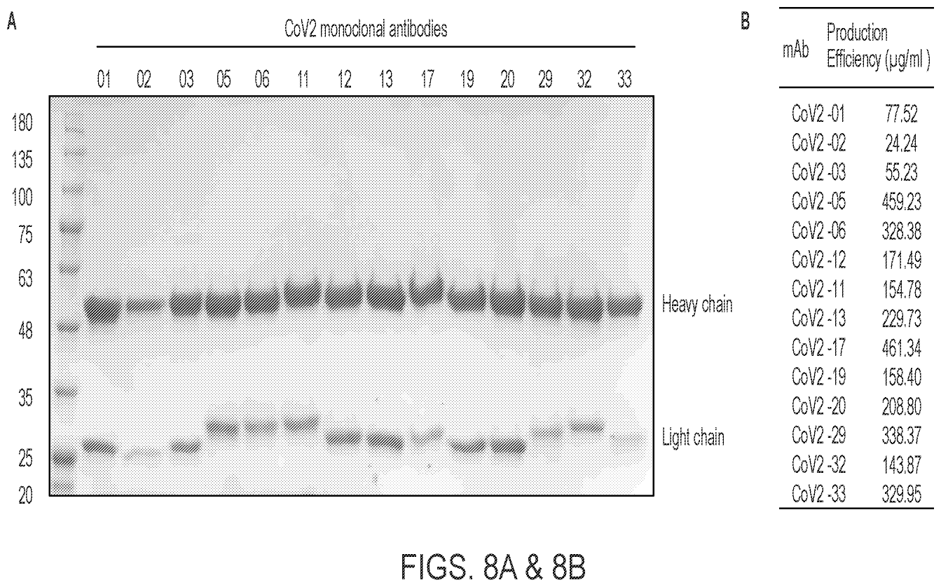

The purity and production efficiency of various antibodies are shown in FIGS. 8A-8B.

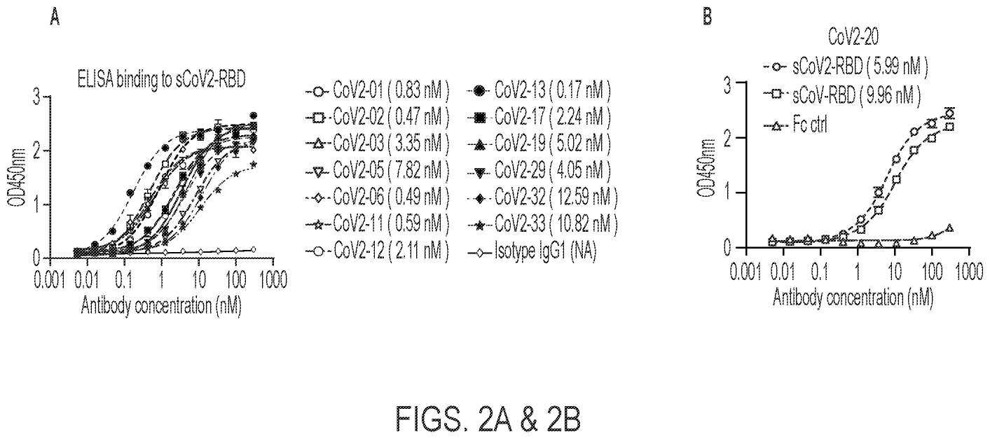

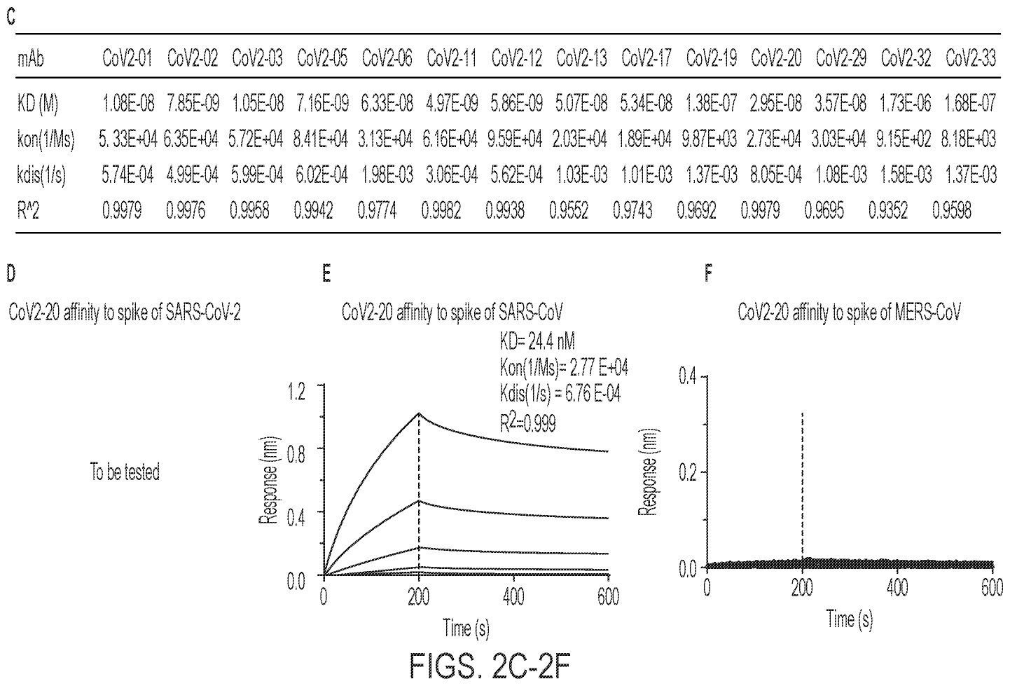

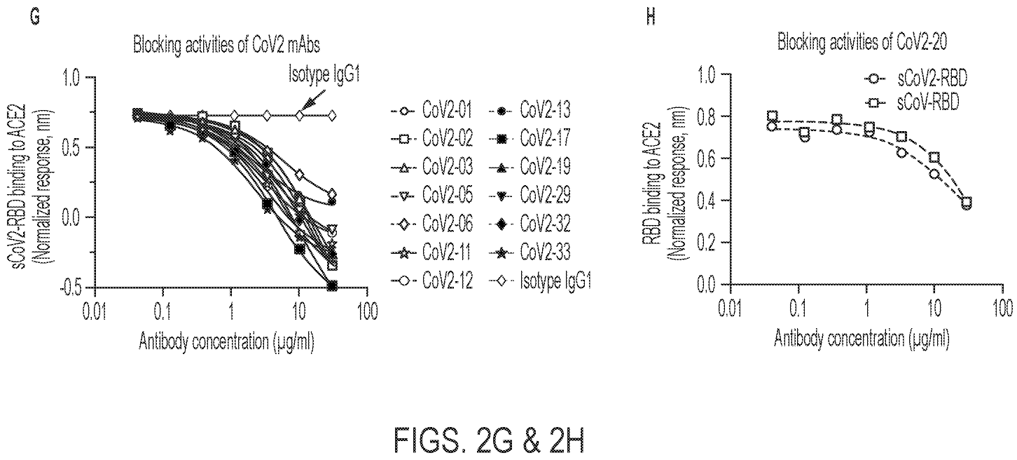

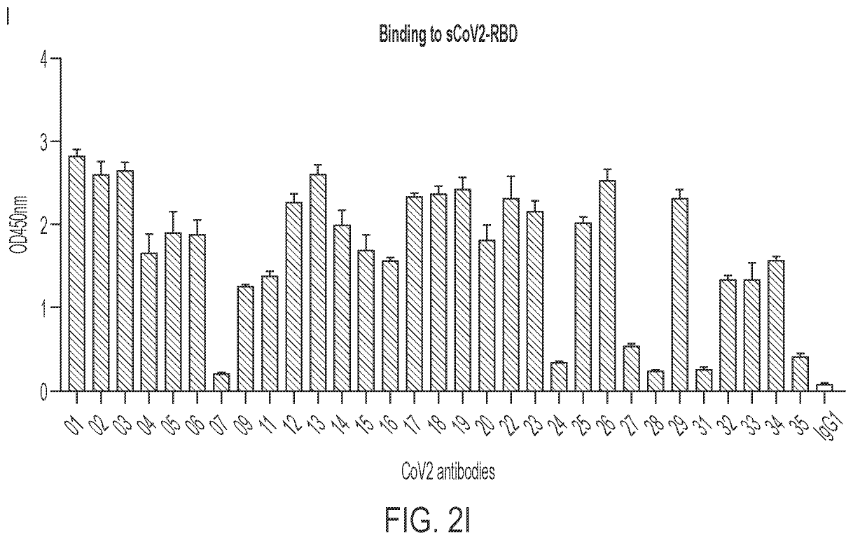

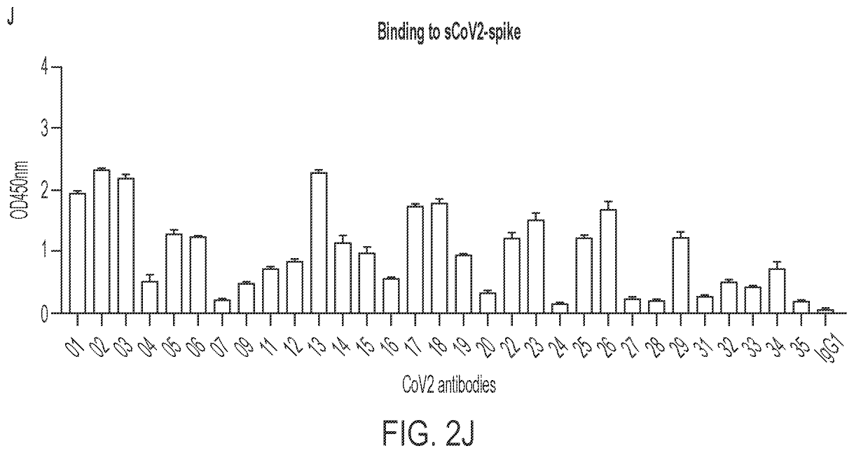

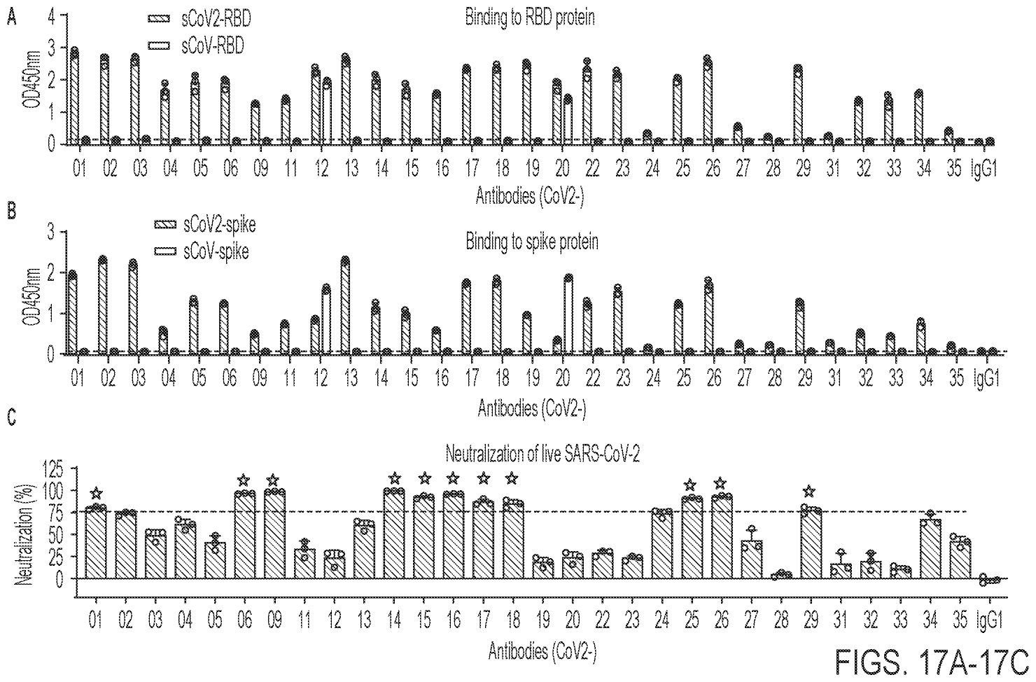

The binding and blocking characteristics of the identified monoclonal antibodies were characterized using ELISA titration to demonstrate the binding of the RBD of SARS-CoV-2 and the EC₅₀ values were determined (see, FIGS. 2A-2J, FIGS. 17A-B, and Table 1).

TABLE 1
EC_{50S} of CoV2-mAbs derived from antibody concentration titration ELISA

| CoV2 | | | | | | | | | | | | | | | |
|---|---|---|---|---|---|---|---|---|---|---|---|---|---|---|---|
| mAb | 01 | 02 | 03 | 05 | 06 | 11 | 12 | 13 | 17 | 19 | 20 | 29 | 32 | 33 | 34 |
| EC50 | 0.83 | 0.47 | 3.35 | 7.82 | 0.49 | 0.60 | 2.11 | 0.17 | 2.24 | 5.02 | 22.0 | 4.05 | 12.59 | 10.82 | 6.67 |
| (nM) | | | | | | | | | | | | | | | |
| CoV2 | | | | | | | | | | | | | | | |
| mAb | 04 | 09 | 14 | 15 | 16 | 18 | 22 | 23 | 24 | 25 | 26 | 27 | 28 | 31 | 35 |
| EC50 | 5.87 | 3.35 | 0.58 | 4.35 | 7.77 | 2.41 | 3.41 | 1.04 | 29.72 | 0.84 | 0.18 | 16.73 | 130.00 | 58.11 | 39.95 |
| (nM) | | | | | | | | | | | | | | | |

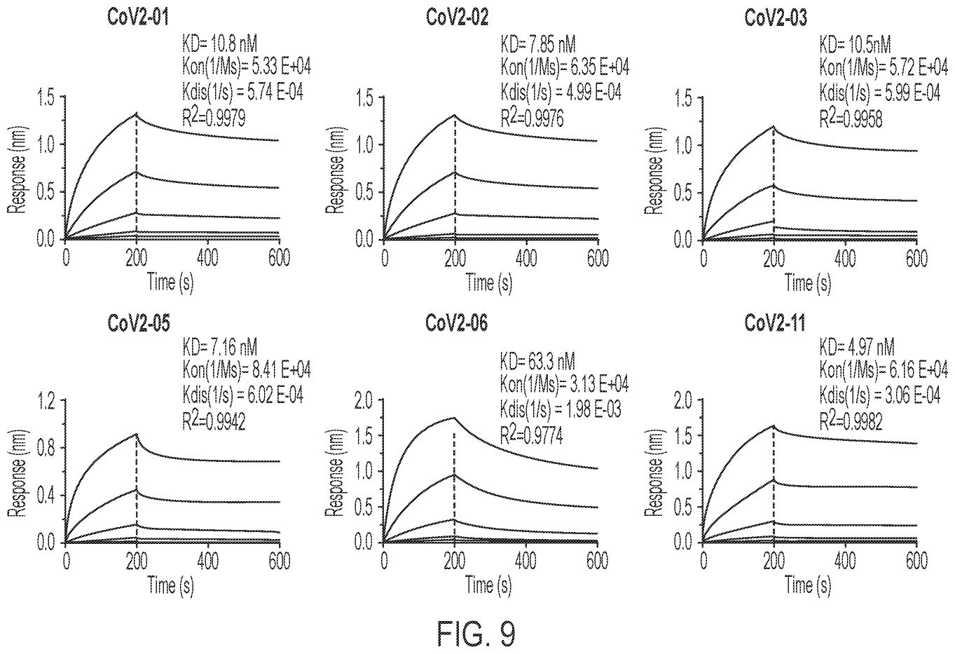

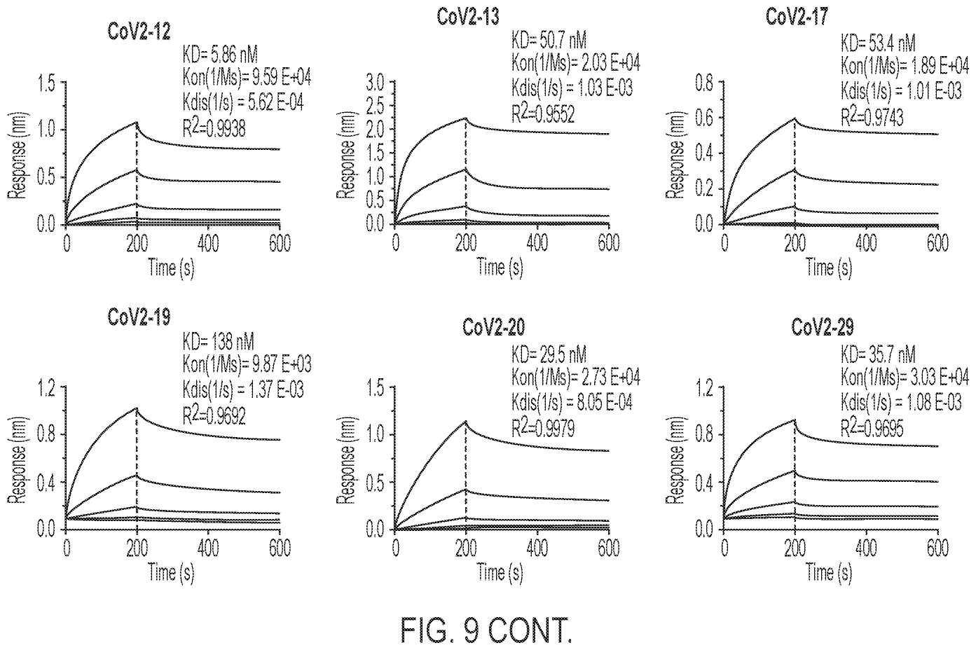

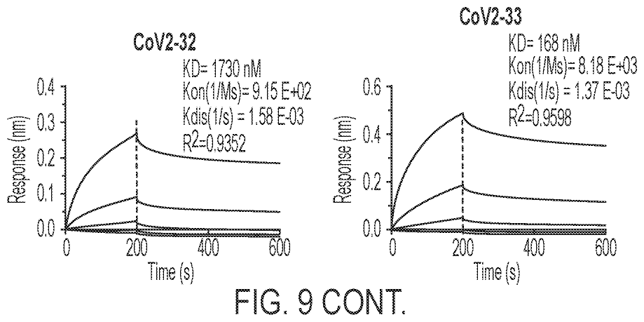

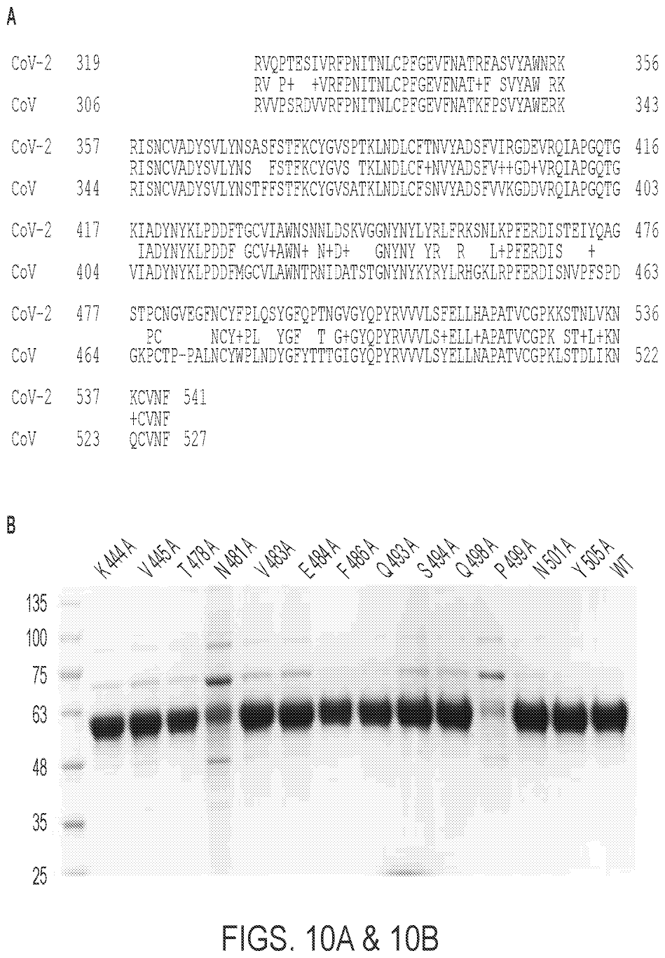

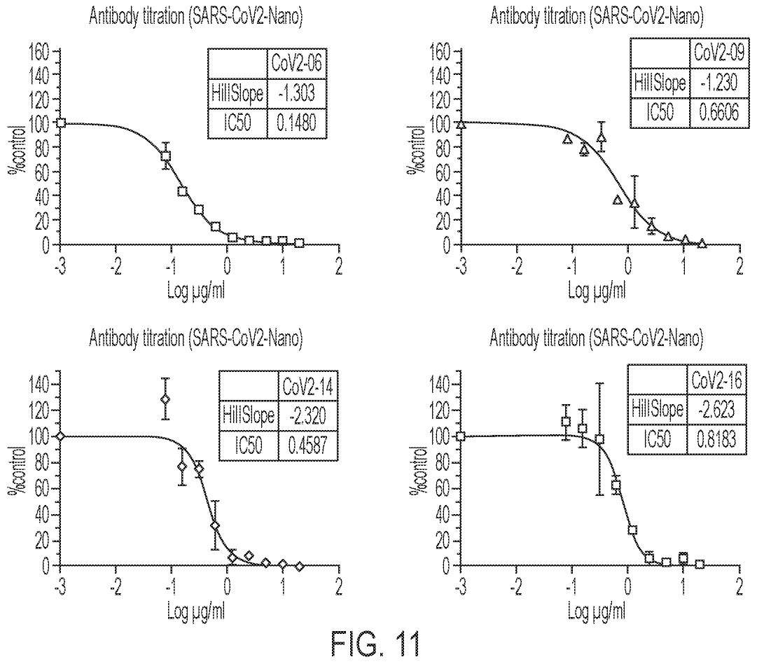

The affinities of the mAbs to the RBD of SARS-CoV2 was also measured. The affinity value KD, association constant value Kon, dissociation constant value Koff and the R2 from kinetic curve fitting were determined. At least one antibody bound significantly to both the RBD of SARS-CoV-2 and that of SARS-CoV. The kinetics and affinities of this mAb for the spike proteins of SARS-CoV-2, SARS-CoV and MERS-CoV were characterized. A sequence comparison of SARS-CoV-2 RBD (amino acids 319 to 541 of SEQ ID NO: 401) and SARS-CoV RBD (amino acids 306 to 527 of SEQ ID NO: 402) is shown in FIG. 10A. The ability of sCoV2-RBD binding to ACE2 by purified sCoV2-RBD binding to ACE2 by purified CoV2 mAbs was demonstrated at different concentrations, as was the blocking of sCoV2-RBD and sCoV-RBD binding to ACE2 by the cross reactive monoclonal at different concentrations (see FIGS. 2A-2J, FIG. 9, FIGS. 17A-B, and Table 2).

TABLE 2
Kinetic binding constants for CoV2-mAbs derived from
sensorgraphs generated using Octet instrument

| mAb | KD (M) | kon(1/Ms) | kdis(1/s) | R\hat{ }2 |
|---|---|---|---|---|
| CoV2-01 | 1.08E−08 | 5.33E+04 | 5.74E−04 | 0.9979 |
| CoV2-02 | 7.85E−09 | 6.35E+04 | 4.99E−04 | 0.9976 |
| CoV2-03 | 1.05E−08 | 5.72E+04 | 5.99E−04 | 0.9958 |
| CoV2-05 | 7.16E−09 | 8.41E+04 | 6.02E−04 | 0.9942 |
| CoV2-06 | 6.33E−08 | 3.13E+04 | 1.98E−03 | 0.9774 |
| CoV2-11 | 4.97E−09 | 6.16E+04 | 3.06E−04 | 0.9982 |
| CoV2-12 | 5.86E−09 | 9.59E+04 | 5.62E−04 | 0.9938 |
| CoV2-13 | 5.07E−08 | 2.03E+04 | 1.03E−03 | 0.9552 |
| CoV2-17 | 5.34E−08 | 1.89E+04 | 1.01E−03 | 0.9743 |
| CoV2-19 | 1.38E−07 | 9.87E+03 | 1.37E−03 | 0.9692 |
| CoV2-20 | 2.95E−08 | 2.73E+04 | 8.05E−04 | 0.9979 |
| CoV2-29 | 3.57E−08 | 3.03E+04 | 1.08E−03 | 0.9695 |
| CoV2-32 | 1.73E−06 | 9.15E+02 | 1.58E−03 | 0.9352 |
| CoV2-33 | 1.68E−07 | 8.18E+03 | 1.37E−03 | 0.9598 |

Studies in FIG. 6 illustrate phage ELISA binding to sCoV-2-RBD and sCoV-RBD by the 376 tested output phage clones

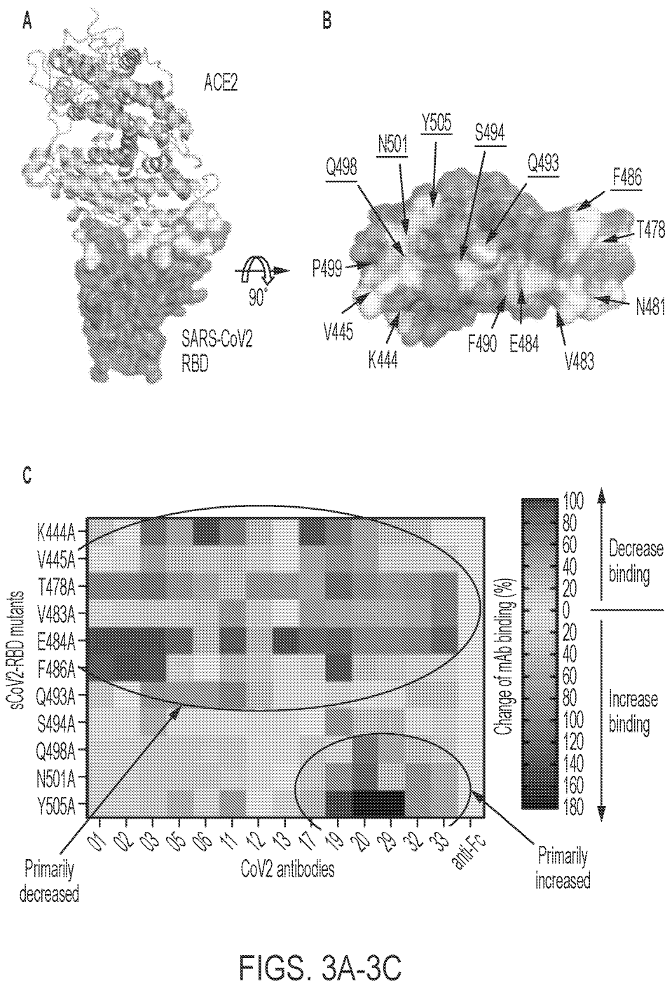

The studies in FIG. 3A illustrate the structure complex of SARS-CoV-2 RBD and ACE2. The RBD is highlighted in purple with the receptor-binding interface highlighted in yellow, and the ACE2 is highlighted in green. The structure was analyzed and depicted using Pymol based on the PDB 2AJF. FIG. 3B is a side view of the SARS-CoV-2 RBD. Fourteen residues within the receptor-binding motif (RBM) that are mutated to Alanine are indicated by arrows. Five residues that directly interact with ACE2 are underlined.

FIG. 3C shows epitope mapping of SARS-CoV-2 mAbs. A heat map of single amino acid mutations on mAb binding to the RBD based on ELISA binding assay. Residues that decrease mAb binding are indicated by a gradient of red colors, and residues that increased mAb binding are by a gradient of blue colors.

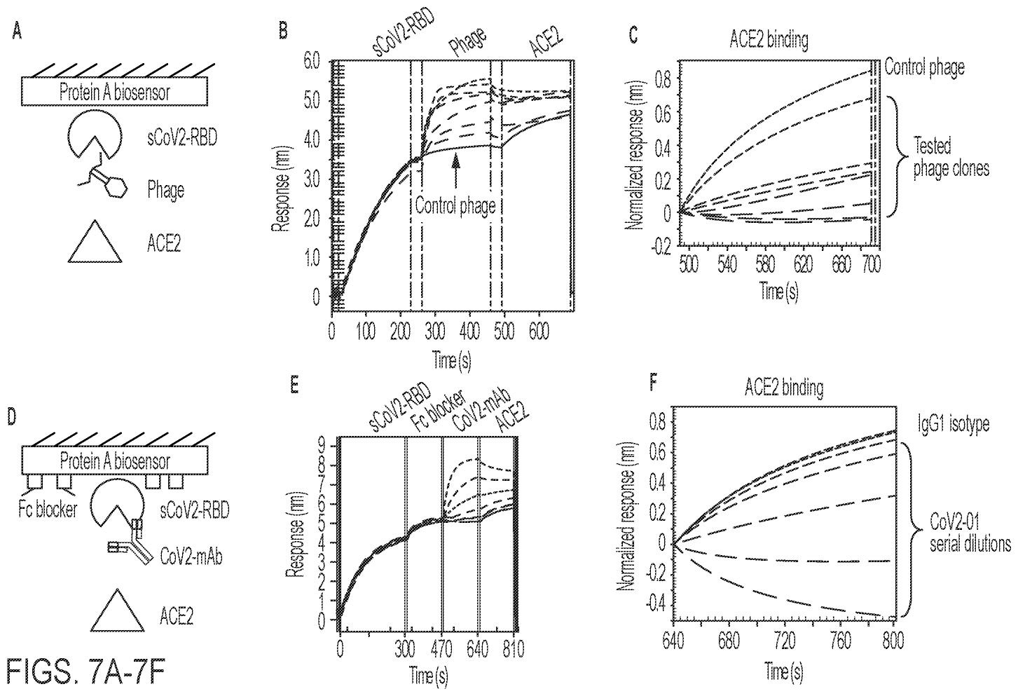

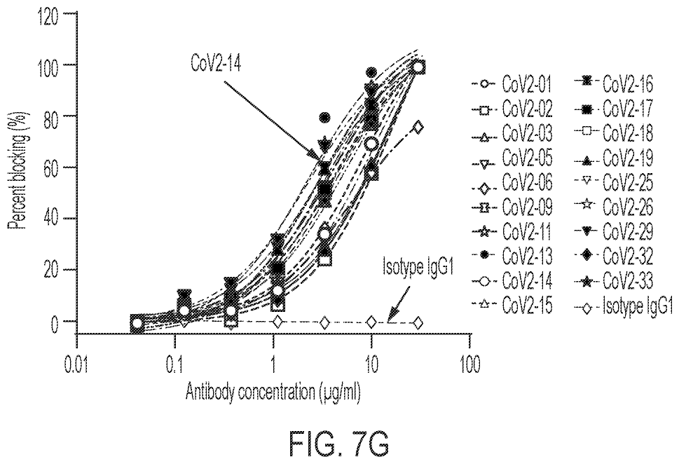

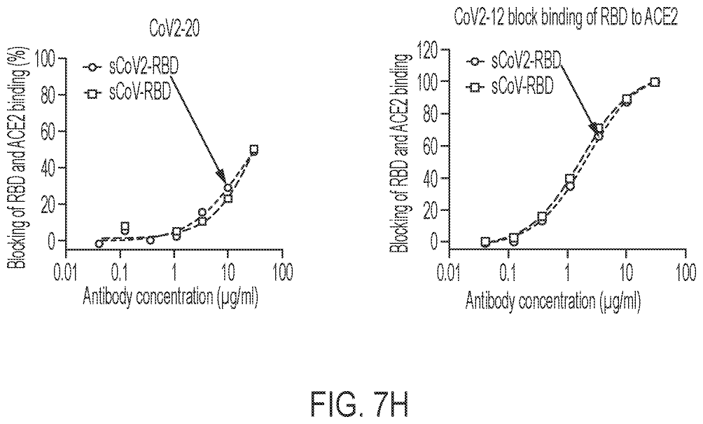

FIGS. 7A-7H and Table 3 illustrate the results when an Octet competition assay was used to identify phage or antibodies that block the binding of sCoV2-RBD to human ACE2

TABLE 3
IC₅₀ of Cov2-mAbs in blocking RBD/ACE2 interaction

| | mAb (CoV2-) | IC50 (μg/ml) |
|---|---|---|
| Blocking of sCoV2-RBD | 01 | 9.17 |
| | 02 | 17.7 |
| | 03 | 14.8 |
| | 05 | 4.18 |
| | 06 | 6.02 |
| | 09 | 3.54 |
| | 11 | 2.97 |
| | 13 | 2.29 |
| | 14 | 3.14 |
| | 15 | 7.68 |
| | 16 | 6.9 |
| | 17 | 4.28 |
| | 18 | 6.03 |
| | 19 | 14.5 |
| | 25 | 3.82 |
| | 26 | 3.25 |
| | 29 | 2.25 |
| | 32 | 10.4 |
| | 33 | 5.47 |
| | 12 | 2.13 |
| | 20 | 14.7 |
| Blocking of sCoV-RBD | 12 | 1.71 |
| | 20 | 44.6 |

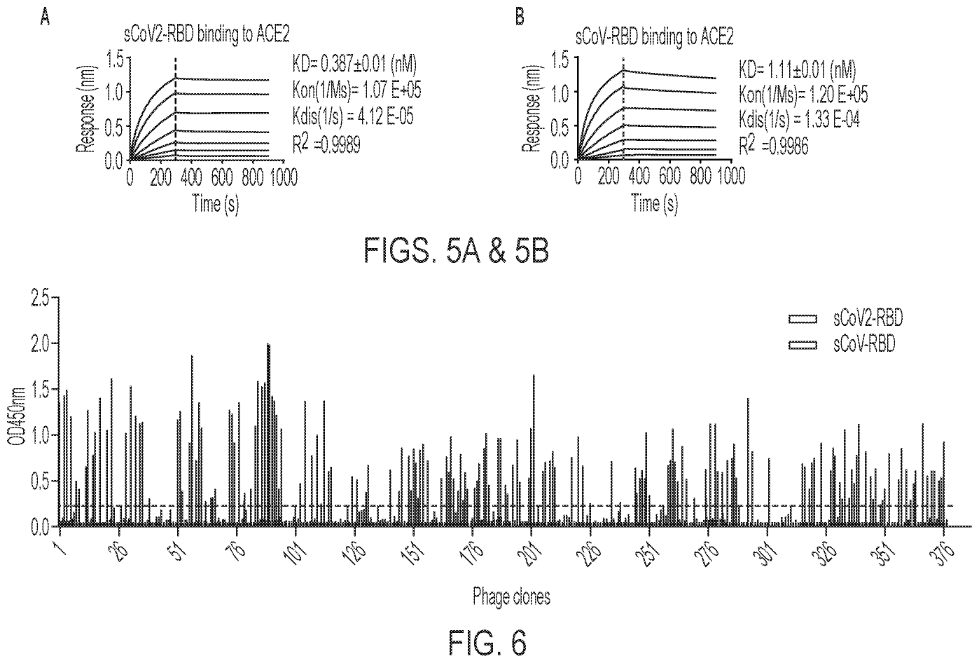

FIGS. 5A-5B show antibody neutralization of SARS-CoV-2 and SARS-CoV. They have identified neutralization titers of the mAbs against SARS-CoV-2 of the mAbs and one mAb that had a significant neutralization titer against SARS-CoV-2 and SARS-CoV. They have also identified that at least one combination of these monoclonal antibodies (mAb) that have synergistic effects and each is from a different epitope bin.

Table 4 illustrates the method and results of the Epitope binning of SARS-CoV-2 mAbs. They also have representative cell images of CoV-2 mAb and isotype mAb using (GFP/CPE). They are investigating anti-viral effects of mAb in combination with small molecules.

TABLE 4
Epitope grouping of Cov2-mAbs

| | Epitope | Key amino acids (numbering relative to SEQ |
|---|---|---|
| mAb (CoV2-) | bins | ID NO: 401) |
| 01 | bin2B/C | T478, E484, F486 |
| 02 | bin2B/C | T478, E484, F486 |
| 03 | bin2B/C | K444, V445, T478, E484, F486, Q493 |
| 05 | bin2A/B | T478, E484, Q493 |
| 06 | bin2A/B | K444, Q493 |
| 09 | bin2B/C | E484 |
| 11 | bin2A/B | K444, V483, E484, Q493, |
| 12 | bin1 | T478 |
| 13 | bin2A/B | T478, E484 |
| 14 | bin2C | E484, F486 |
| 15 | bin2C | E484, F486 |
| 16 | bin2B/C | E484 |
| 17 | bin2A | K444, V483, E484 |
| 18 | bin2B/C | E484 |
| 19 | bin2B/C | K444, V445, T478, E484, F486, S494 |
| 20 | bin1 | K444, V445, T478, E484 |
| 25 | bin2C | V483, F486 |
| 26 | bin2A/B | F486 |
| 29 | bin3 | T478, E484 |
| 32 | bin3 | T478, V483, E484 |
| 33 | bin3 | T478, V483, E484 |

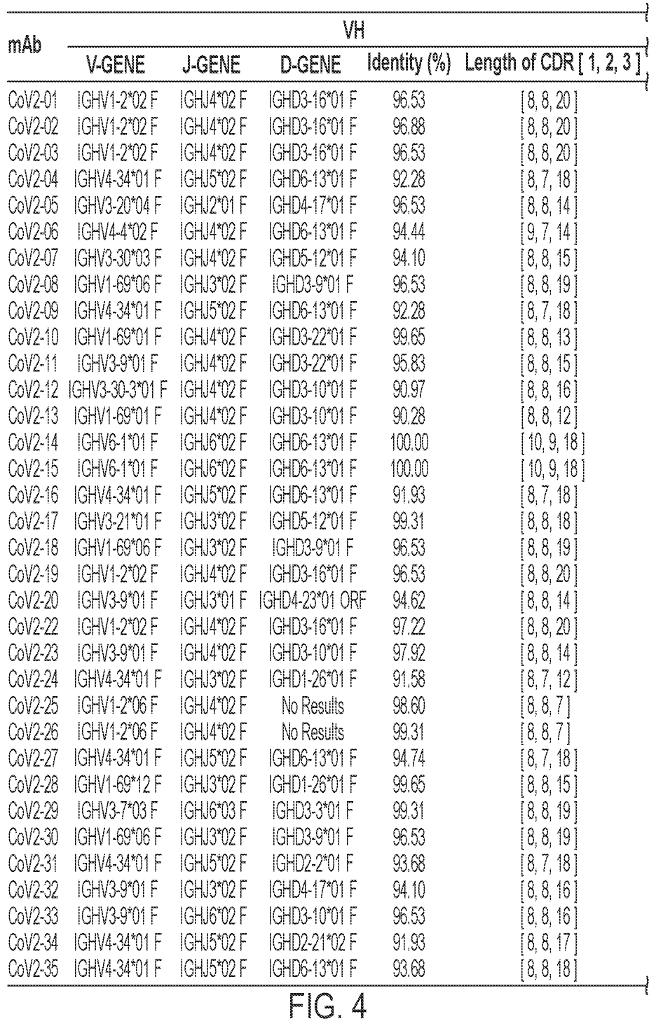

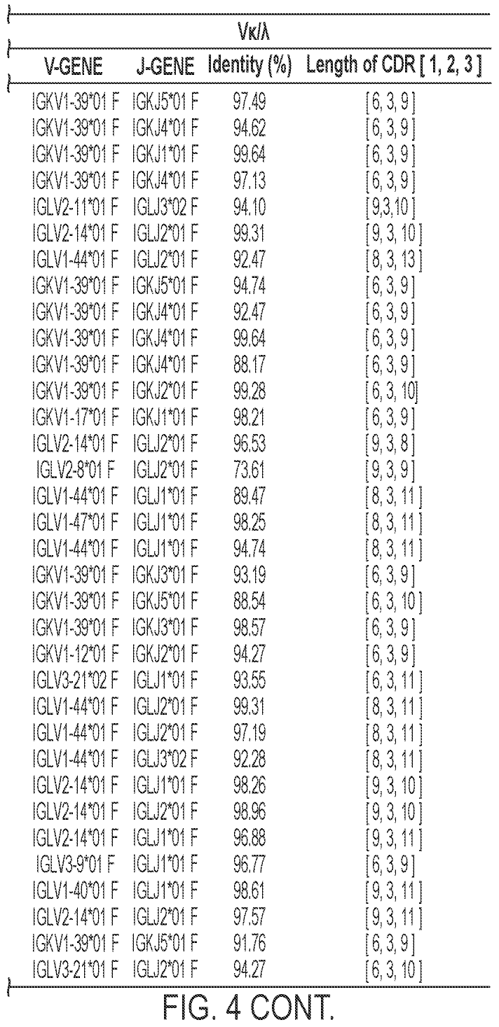

In FIG. 4, the group has also identified the germline gene origins, V-region identity and CDR length of SARS-CoV-2 mAbs they have characterized.

FIG. 10B illustrates the expression and purity of sCoV2-RBD proteins with single residue mutations.

### Example 2—Neutralization Assays with Antibodies

A neutralization assay was developed (see, e.g., Xie et al., 2020, incorporated herein by reference) and employed to assess the neutralizing activity of antibodies of the embodiments. Table 5 below summarizes the neutralizing antibody titers using a reporter SARS-CoV-2. Briefly, a reporter SARS-CoV-2 (engineered with a mNeonGreen reporter gene) was incubated with serially diluted antibodies at 37° C. for 1 hour. The mixtures of virus and antibody were used to infect Vero E6 cells. At 24 hours post infection, the cells were quantified for mNeonGreen reporter signals. The efficacy of antibody to block reporter virus infection was quantified by the EC₅₀ values (concentrations required to inhibit 50% of SARS-CoV-2 Infection).

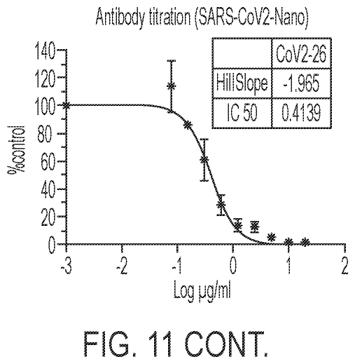

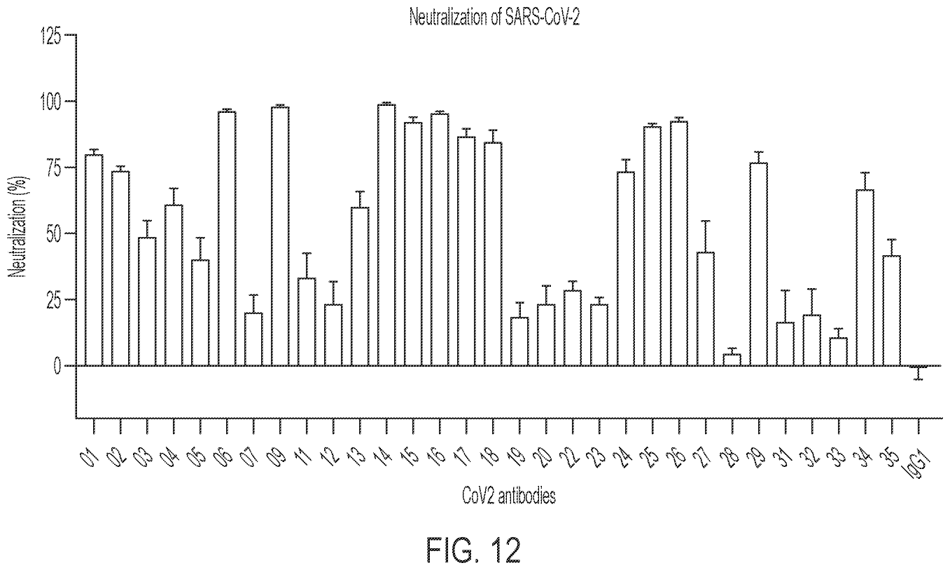

Using this cell-based assay as described and antibody concentrations were titrated from 20 μg/ml at 3-fold down to determine the neutralization potency. The neutralizing potency of Cov2-mAbs in the reporter virus infection assay was quantified by the NC₅₀ values (concentrations required to inhibit 50% of SARS-CoV-2 Infection) are detailed in FIG. 11, FIG. 12, and FIG. 17C.

TABLE 5
NC₅₀ VALUES

| Sample ID | NC₅₀ (μg/ml) |
|---|---|
| CoV2-01 | 5.2 |
| CoV2-06 | 0.15 |
| CoV2-09 | 0.66 |
| CoV2-12 | 18.2 |
| CoV2-14 | 0.46 |
| CoV2-15 | 1.15 |
| CoV2-16 | 0.82 |
| CoV2-17 | 2.49 |
| CoV2-18 | 1.93 |
| CoV2-25 | 1.24 |
| CoV2-26 | 0.41 |
| CoV2-29 | 1.13 |

### Example 3—Materials and Methods

Expression and purification of RBD proteins. The receptor-binding domain (RBD) (R319-F541 of SEQ ID NO: 401) of the spike protein of SARS-CoV-2 (UNIPROT KD: PODTC2, SEQ ID NO: 401) (Gene Bank nucleotide sequence of isolate Wuhan-Hu-1: MN908947.3 encodes SEQ ID NO: 401 and has an RBD identical to that shown in FIG. 10A) and the RBD (R306-F527 of SEQ ID NO: 402) of the spike protein of SARS-CoV (GenBank: AAP51227.1, SEQ ID NO: 402) were fused with a human IgG1 Fc fragment and inserted into expression vectors. The constructed plasmids were transiently transfected into Expi293F cells for protein expression. After six days, the culture supernatants were harvested, and the proteins were affinity purified using Protein A resin. The proteins were named as sCoV2-RBD and sCoV-RBD, respectively. The protein purities were assessed by SDS-PAGE, and their binding activities to ACE2 were tested by a Bio-Layer interferometry (BLI) assay. The sCoV2-RBD proteins with mutations were generated by the same method. For comparison of protein expressing level, plasmids expressing wild-type or mutant sCoV2-RBD proteins were used to transfect Expi293F cells in triplicates, after 4 days of transfection, the cell supernatant were harvested and the protein concentrations were quantitated on the Octet RED96 system.

Phage library panning and selection of mAbs targeting the RBD. The sCoV2-RBD protein was used for antibody selection by panning a large human scFv phage display antibody library (containing ˜10¹² antibodies). The library was constructed in house from the cDNA extracted from the PBMCs and tonsils of multiple donors (Zhao et al., 2019). In each round of phage panning, 50 μg of sCoV2-RBD was coated on a MaxiSorp immune tube and blocked by 8% milk. The phages were pre-blocked by 8% milk and then pre-absorbed by an Fc antigen for deselection. The pre-blocked and deselected phages were then incubated with the antigen pre-coated on the immune tube. After washing with PBST and PBS, the phages were eluted by triethylamine (TEA). The eluates were tittered and infected E. coli TG1 for phage amplification for next round of panning Similar procedures were performed in round 2 of panning with increased washing stringency. After 2 rounds of panning, the phage eluates were used to infect E. coli TG1 to grow single colonies for picking by QPix420 system (Molecule Devices) and for phage preparation. Individual phage clones were tested for ELISA binding to sCoV2-RBD, sCoV-RBD and Fc control. The HRP-conjugated Mouse-anti-M13 antibody (Santa Cruz, #sc-53004 HRP) was used for detection of antigen-bound phages. The sCoV2-RBD positive clones were sequenced for their scFv sequences to obtain unique phage binders.

DNA sequencing, germline gene analysis of antibodies. The phagemids of sCoV2-RBD positive phage clones were prepared by QIAGEN BioRobot 8000 and sequenced in the scFv region using a specific primer (Table 7). Online IMGT/V-QUEST analysis of antibody sequences resulted in the report of the germline genes origins, the V-region identities, and the length of CDR for the VH and Vic/k.

Expression and purification of antibodies. After sequence analysis, the VH and Vκ/λ were PCR amplified and inserted into the IgG1 heavy chain and corresponding light chain backbones. The plasmids were transfected into Expi293F cells and cultured for 7 days. The supernatants were collected, and antibodies were purified using Protein A resin. All the antibody preparations were reconstituted in PBS buffer for the studies.

Neutralization assay with live SARS-CoV-2. The neutralization assay for the 30 antibodies at 10 μg/ml, neutralization titration assay for the 11 neutralizing antibodies, and the synergistic neutralization assay were performed using the SARS-CoV-2-mNG virus generated before (Xie et al., 2020). A total of 1.5×10⁴ Vero cells in phenol red-free culture medium were plated into each well of a black transparent flat-bottom 96-well plate (Greiner Bio-One; Cat #655090). On the next day, antibodies (single dilution or 2-fold serial dilutions) were mixed with an equal volume of SARS-CoV-2-mNG virus (MOI=0.5) After 1 h incubation at 37° C., the antibody-virus complexes were inoculated into the 96-well plate containing confluent Vero cells. The infections were performed in duplicates or triplicates. At 20 h post-infection, nuclei were stained by the addition of Hoechst 33342 (Thermo Fisher Scientific) to a final concentration of 10 nM. Fluorescent images were acquired using a Cytation 7 multi-mode reader (BioTek). Total cells (in blue) and mNG-positive cells (in green) were counted, and the infection rate was calculated. The relative infection rates were calculated by normalizing the infection rate of each well to that of control wells (no antibody treatment). The relative infection rate versus the log 10 value of the concentration was plotted, and the 50% neutralization concentration (NT 50) was obtained by using a four-parameter logistic regression model from the GraphPad Prism 8 software.

The activity for the most potent neutralizing antibody CoV2-06 was validated in another live virus assay using wild-type SARS-CoV-2 (Isolate USA/WA1/2020) in Vero-E6 cells. The Antibody was subjected to two-fold dilutions in DMEM 2% FBS from 12.5 μg/ml to 0.048 μg/ml and mixed with 10 TCID₅₀s of SARS-CoV-2 in 96-well plates. Eight replicative wells were set for each antibody concentration. After incubation for 1 h at 37° C., the mixtures were added to 6,000 Vero cells for incubation. After 6 days, the cytopathic effect (CPE) of cells in each well was visually checked under microscopy and the percentages of wells showing CPE were recorded.

Neutralization synergy analysis. The Chou-Talalay method was used to analyze the cooperation of antibody in neutralization (Chou & Talalay, 1984). The CoV2-06 and CoV2-14 were combined (mass ratio of CoV2-06 to CoV2-14 is 1:3) based on their NT₅₀ values. Then, antibody alone or in mixture were 2-fold diluted to cover multiple doses above and below NT₅₀s. The neutralization percentages at each dose were entered as fraction affected (Fa, ranging from 0.01 to 0.99) into the CompuSyn software (world-wide-web at combosyn.com/index.html). The dose-effect curves were generated, and Combination Index (CI) at ED₅₀ (50% effective dose), ED₇₅, ED₉₀ and ED₉₅ were calculated based on the Fa-CI plots. CI<1, synergism; CI=1, additive effect; CI>1, antagonism.

SARS-CoV-2 S pseudovirus neutralization assay. For preparation of pseudovirus, the SARS-CoV-2 S expressing 293T cells were infected with VSV-G pseudotyped VSV_{Δ}G-RFP-, a replication-defective virus encoding a red fluorescent protein reporter in the place of the VSV G glycoprotein. Vero E6 cells stably expressing TMPRSS2 were seeded in 100 μL at 2.5×10⁴ cells/well in a 96 well collagen coated plate. The next day, 2-fold serially diluted antibody at a starting concentration of 10 μg/ml was mixed with VSVAG-RFP SARS-CoV-2 pseudotype virus (˜150 focus forming units/well) and incubated for 1 h at 37° C. Also included in this mixture to neutralize any potential VSV-G carryover virus was 8G5F (Zost et al., 2020a) 11, a mouse anti-VSV Indiana G, at a concentration of 100 ng/ml (Absolute Antibody, Boston, MA). The antibody-virus mixture was then used to replace the media on VeroE6 TMPRSS2 cells. 20 h post infection, the cells were washed and fixed with 4% paraformaldehyde before visualization on an S6 FluoroSpot Analyzer (CTL, Shaker Heights OH). Individual infected foci were enumerated, and the values compared to control wells without antibody.

ELISA titration of mAb binding and fitting of EC₅₀. Corning high binding assay plates were coated with recombinant sCoV2-RBD or sCoV-RBD protein (1 μg/ml) at 4° C. overnight and blocked with 5% skim milk at 37° C. for 2 hours. Serially diluted antibodies were added at a volume of 100 μl per well for incubation at 37° C. for 2 h. The anti-human IgG Fab2 HRP-conjugated antibody was diluted 1:5000 and added at a volume of 100 μl per well for incubation at 37° C. for 1 h. The plates were washed 3-5 times with PBST (0.05% Tween-20) between incubation steps. TMB substrate was added 100 μl per well for color development for 3 mins and 2M H₂SO₄ was added 50 μl per well to stop the reaction. The OD₄₅₀ₙₘ was read by a SpectraMax microplate reader. The data points were plotted by GraphPad Prism8, and the EC₅₀ values were calculated using a three-parameter nonlinear model.

Bio-layer interferometry (BLI) measurement of affinity. Antibody affinity was measured on Pall ForteBio Octet RED96 system. Recombinant antibodies (20 μg/ml) was loaded onto the Protein A biosensors for 300 seconds. Following 10 seconds of baseline in kinetics buffer, the loaded biosensors were dipped into serially diluted (0.14-300 nM) RBD protein (Sino Biological, Cat: 40592-V08B) or the previously generated spike protein for 200 seconds to record association kinetics (Wrapp et al., 2020). The sensors were then dipped into a kinetic buffer for 400 seconds to record dissociation kinetics. Kinetic buffer without antigen was set to correct the background. The Octet Data Acquisition 9.0 was used to collect affinity data. For fitting of K_{D} value, Octet Data Analysis software V11.1 was used to fit the curve by a 1:1 binding model and use the global fitting method. Similarly, binding affinities to ACE2 by wild-type sCoV2-RBD protein, mutant sCoV2-RBD proteins, and sCoV-RBD protein were measured.

Epitope binning of antibodies. Epitope binning was performed on the octet RED96 system using a sandwich format. Briefly, individual antibodies (1^{st} antibodies) were diluted to 50 μg/ml and loaded onto Protein A biosensors. After blocking with 200 μg/ml of an irrelevant IgG1, the sensors were dipped into 15 μg/ml of His tagged sCoV2-RBD to capture the antigen. The sensors with antibody-antigen complex were then incubated with the rest antibodies (2n d antibodies) pairwise in each round of binning. In each round of binning, an isotype IgG1 was used as a control. A total of 15×15 sets of antibody binning were performed to obtain the full profile of antibody epitope bins. For data analysis, if a 2n d antibody still bind the RBD pre-captured by a 1^{st} antibody, the 1^{st} antibody was defined as competitive with the 2^{nd} antibody; if a 2^{nd} antibody did not bind the RBD pre-captured by a 2^{nd} antibody, the Pt antibody was defined as non-competitive with the 2^{nd} antibody. The antibody pairs with competition were grouped and defined as the same bin.

Epitope mapping of antibodies. Epitope mapping was performed using a SARS-CoV-2 (strain Wuhan-Hu-1, QHD43416.1) S protein RBD shotgun mutagenesis mutation library (Davidson & Doranz, 2014). A full-length expression construct for S protein, where 184 residues of the RBD (between residues corresponding to amino acids 335-526 of SEQ ID NO: 401) were individually mutated to alanine, and alanine residues to serine. Mutations were confirmed by DNA sequencing, and clones arrayed in a 384-well plate, one mutant per well. Binding of mAbs to each mutant clone in the alanine scanning library was determined, in duplicate, by high-throughput flow cytometry. Each S protein mutant was transfected into HEK-293T cells and allowed to express for 22 hrs. Cells were fixed in 4% (v/v) paraformaldehyde (Electron Microscopy Sciences), and permeabilized with (w/v) saponin (Sigma-Aldrich) in PBS plus calcium and magnesium (PBS++) before incubation with mAbs diluted in PBS++, 10% normal goat serum (Sigma), and 0.1% saponin. The mAb screening concentrations were determined using an independent immunofluorescence titration curve against cells expressing wild-type S protein to ensure that signals were within the linear range of detection. Antibodies were detected using 3.75 μg/mL of AlexaFluor488-conjugated secondary antibody in 10% normal goat serum with saponin. Cells were washed three times with PBS++/0.1% saponin followed by two washes in PBS and mean cellular fluorescence was detected using a high-throughput Intellicyte iQue flow cytometer (Sartorius). Antibody reactivity against each mutant S protein clone was calculated relative to wild-type S protein reactivity by subtracting the signal from mock-transfected controls and normalizing to the signal from wild-type S-transfected controls. Mutations within clones were identified as critical to the mAb epitope if they did not support reactivity of the test mAb but supported reactivity of other SARS-CoV-2 antibodies. This counter-screen strategy facilitates the exclusion of S mutants that are locally misfolded or have an expression defect. Validated critical residues represent amino acids whose side chains make the highest energetic contributions to the mAb-epitope interaction (Bogan and Thorn, 1998; Lo Conte et al., 1999).

For further validations of some of the critical residues identified, the sCoV2-RBD proteins with specific point mutations were generated to test antibody reactivity. Briefly, the wild-type and mutant sCoV2-RBD were coated on ELISA plates and tested for binding for all the mAbs at 3 μg/ml. HRP-conjugated goat-anti-human IgG1 Fc was used as a positive control and to normalize the OD₄₅₀ₙₘ values among the mutant and wild-type sCoV2-RBD proteins. The relative binding of each mutant to WT was calculated as (%)=OD₄₅₀ₙₘ of mutant protein/OD₄₅₀ₙₘ of wild-type protein*100%.

Octet based assay for antibody blocking of RBD and ACE2 interaction. The purified antibodies were tested for their blocking activities against sCoV2-RBD binding to ACE2 on the Octet RED96 system. The sCoV2-RBD (5 μg/ml) was captured on the Protein A biosensors for 300 seconds. After capture, the biosensors were blocked by 200 μg/ml of Fc protein and then dipped into serial diluted antibody solutions (0-30 μg/ml) for 200 seconds, and then into ACE2 solution (5 μg/ml) for 200 seconds. Between each incubation step, there were 10 seconds of baseline steps. The binding responses were recorded for all incubation steps. To calculate the percent of blocking, the responses of ACE2 binding were first normalized to the beginning point and then normalized against the smallest response value for each antibody set. The percent of blocking was calculated as blocking (%)=(Normalized response of buffer−Normalized response of mAb)/OD₄₅₀ₙₘ Normalized response of buffer*100%.

Virus escape from neutralizing antibodies. The SARS-CoV-2-mNG virus and Vero E6 cells were used to select neutralization-escape mutant under individual CoV2-06, CoV2-14 or CoV2-06+CoV2-14 for three rounds. Each selection was performed in four replicative wells in a 12-well format. For the first round of selection, 3×10⁵ cells were seeded one day prior to infection. One the next day, 6×10⁵ pfu of virus was pre-incubated with CoV2-06 (10 μg/ml), CoV2-14 (10 μg/ml) or CoV2-06 (1.4 μg/ml)+CoV2-14 (4.1 μg/ml) and the mixtures were added to cells for incubation for 3 days. The supernatants were harvested as round 1 (R1) virus. For the second round of selection, 200 μl of R1 virus was pre-incubated with CoV2-06 (20 μg/ml), CoV2-14 (20 μg/ml) or CoV2-06 (2.8 μg/ml)+CoV2-14 (8.2 μg/ml) and added to cells for incubation for 2-4 days to generate the R2 virus. For the third round of selection, 50 μl of R2 virus was pre-incubated with CoV2-06 (200 μg/ml), CoV2-14 (200 μg/ml) or CoV2-06 (14 μg/ml)+CoV2-14 (41 μg/ml) and added to cells for incubation for 2-4 days to generate the R3 virus. The expressions of mNG were monitored at each round for indication of infection. After three rounds of selections, the R3 virus for each group was Sanger sequenced of the S region using specific primers (Table 7). Antibody neutralizations against the mutant virus were performed as described above.

Size-exclusion chromatography (SEC) analysis. The SEC analysis of wild-type and mutant sCoV2-RBD proteins were performed on the ÄKTA pure system with the Superpose 6 increase 10/300GL column in PBS buffer. All the proteins preparations were centrifuged at 13,800×g for 5 mins to remove visible aggregates and 100 μg of proteins were used for each loading. The collagen, IgG and BSA were used as molecular markers to indicate the retention volumes. The UNICORN 7.0 software was used to analyze and export the data of each curve.

Bioinformatics analysis of the RBD sequences of SARS-CoV-2 clinical isolates. As of Jul. 23, 2020, 70,934 human SARS-CoV-2 genomics sequences and information were collected from GISAID. The viral genomes were downloaded and used as query to search against the reference sequence of the RBD (Gene Bank: MN908947.3) via blastx. The RBD region of the download viral genomes were subsequently gathered via “blastdbcmd” option in ncbi-blast-2.2.30 (ftp.ncbi.nlm.nih.gov/blast/executables/blast+/2.2.30/). Those coding nucleotides were subsequently translated via standalone version of Orf-Predictor (Min et al., 2005). The predicted protein sequences were aligned and variants were determined via standalone version of Clustal Omega.

Animal studies. This study was carried out in accordance with the recommendations for care and use of animals by the Office of Laboratory Animal Welfare, National Institutes of Health. The Institutional Animal Care and Use Committee (IACUC) of University of Texas Medical Branch (UTMB) approved the animal studies under protocol 1802011. Ten- to twelve-week old female BALB/c mice were purchased from Charles River Laboratories and maintained in Sealsafe™ HEPA-filtered air in/out units. A mouse-adapted virus was generated based on a previously reported study Animals were anesthetized with isoflurane and infected intranasally (IN) with 10⁴ pfu of mouse-adapted SARS-CoV-2 in 50 μl of phosphate-buffered saline (PBS). Antibodies were intraperitoneally injected (20 mg/kg or 5 mg/kg) at 16 hours before or 6 hours after viral infection. As a control group, mice were injected with an IgG1 before or after viral infection. Two days after infection, lung samples of infected mice were harvested and homogenized in 1 ml PBS for analysis of infectious virus by plaque assay. The virus harvested from each mice in different antibody treatment (5 mg/kg) groups were individually sequenced of the RBD region using specific primers (Table 7).

Statistical analysis. The statistics for ELISA binding, virus neutralization, and receptor blocking and protein expression levels were calculated with Graphpad Prism 8. The statistics for antibody affinities to the RBD and the RBD to ACE2 were reported with the ForteBio's data analysis software. The combination effects of antibody cocktail was analyzed using the CompuSyn program.

### Example 4—Results

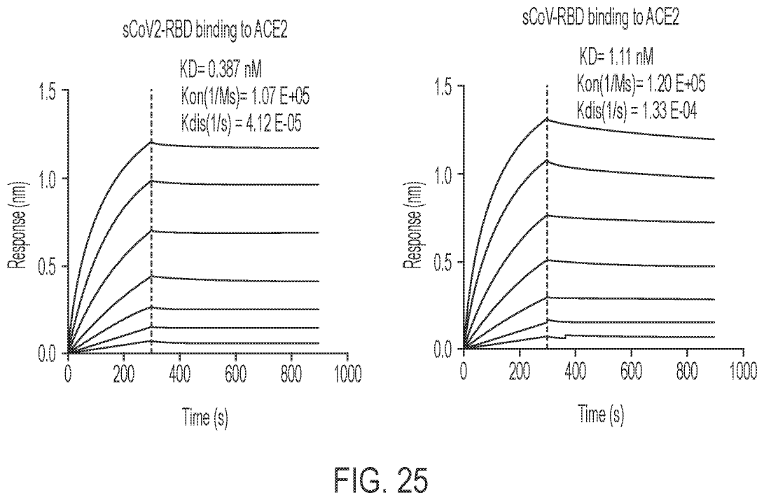

Isolation of RBD-directed human mAbs with potent neutralization of SARS-CoV-2. Since the RBD of SARS-CoV-2 S protein is a crucial antibody target, the inventors focused on isolating RBD-specific mAbs from a single-chain variable fragment (scFv) phage display antibody library. They generated a highly purified Fc-tagged RBD of SARS-CoV-2 (sCoV2-RBD) protein as bait for phage panning They also prepared the RBD of SARS-CoV (sCoV-RBD) for the evaluation of cross-reactivity (FIGS. 1A-1B). The purified RBD proteins bind ACE2 with high affinity (FIG. 25), indicating that these proteins retain the correct conformations. The inventors used sequential panning rounds of a highly diverse naïve scFv phage library with increased stringency to select sCoV2-RBD bound phages. The output phages were analyzed for antigen binding by ELISA. Unique scFv clones were identified by sequencing and converted to full human immunoglobulin G1 (IgG1). After the panning and selection process, 30 mAbs were obtained (FIG. 1C). Among the 30 sCoV2-RBD binding mAbs, two mAbs (CoV2-12 and CoV2-20) show cross-binding to sCoV-RBD (FIG. 17A). All 30 mAbs also bind to the trimeric prefusion S protein of SARS-CoV-2 (FIG. 17B). Cross-binding of CoV2-12 and CoV2-20 to the S of SARS-CoV was confirmed (FIG. 17B). The inventors next screened the 30 mAbs for neutralization of a live SARS-CoV-2 virus engineered with the mNeonGreen marker (Paul-Pletzer, K., 2006). Among them, 11 mAbs achieved >75% neutralization at 10 μg/ml (NT₇₅<10 μg/ml) and the remaining 19 mAbs exhibited <75% neutralization at 10 μg/ml (NT₇₅>10 μg/ml) (FIG. 17C).

The inventors analyzed the germline genes for the variable heavy (VH) and

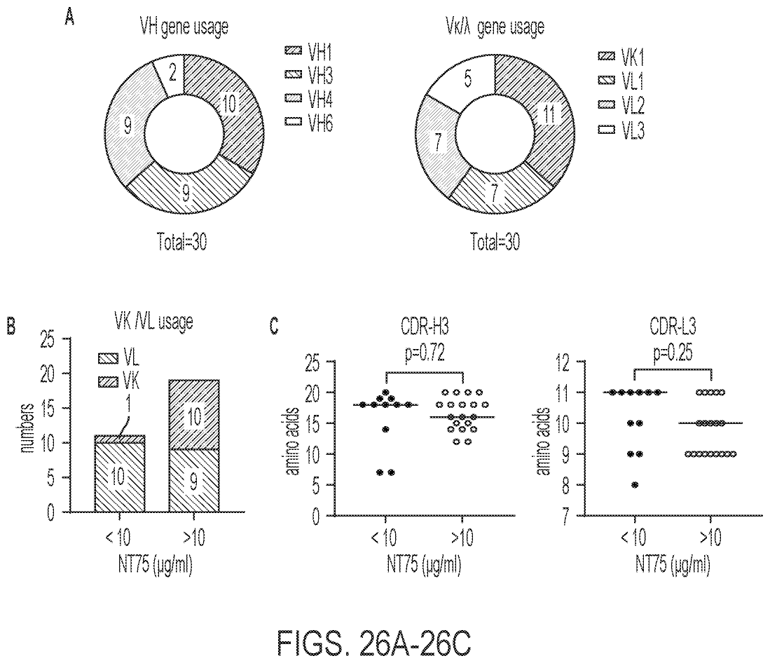

variable light (VL) regions of the 30 mAbs (FIG. 4 and FIGS. 26A-C). The VHs fall within four different gene classes: VH1, VH3, VH4, and VH6. The VHs originated from 13 gene alleles. Notably, the VH of CoV2-14 and CoV2-15 had 100% homology to the original human germline sequence, indicating no somatic mutations (FIG. 4). The VLs also fall within four gene classes: the VK1, VL1, VL2, and VL3. The VLs originated from 12 gene alleles (FIG. 4). The VLs show a bias toward the lambda over kappa usage (FIG. 26A); the typical distribution of human IgG antibodies has a 2:1 ratio of kappa:lambda light chain usage. The inventors further divided the 30 mAbs into two groups based on their NT 75 values (FIG. 17C). They compared the gene usage and CDR3 length of these two groups (FIGS. 26B-C). Significantly, the group of mAbs with NT₇₅<10 μg/ml had a bias toward lambda light chain usage (91% vs. 42% for mAbs with NT₇₅>10 μg/ml) (FIG. 26B) No significant difference between the CDR3 lengths of the two groups was detected (FIG. 26C).

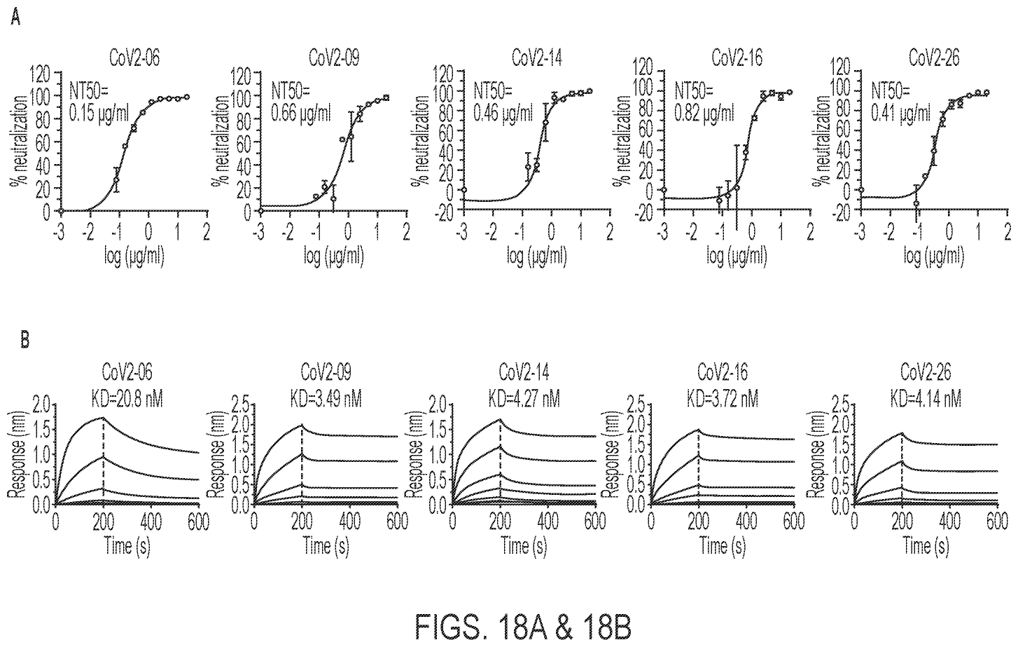

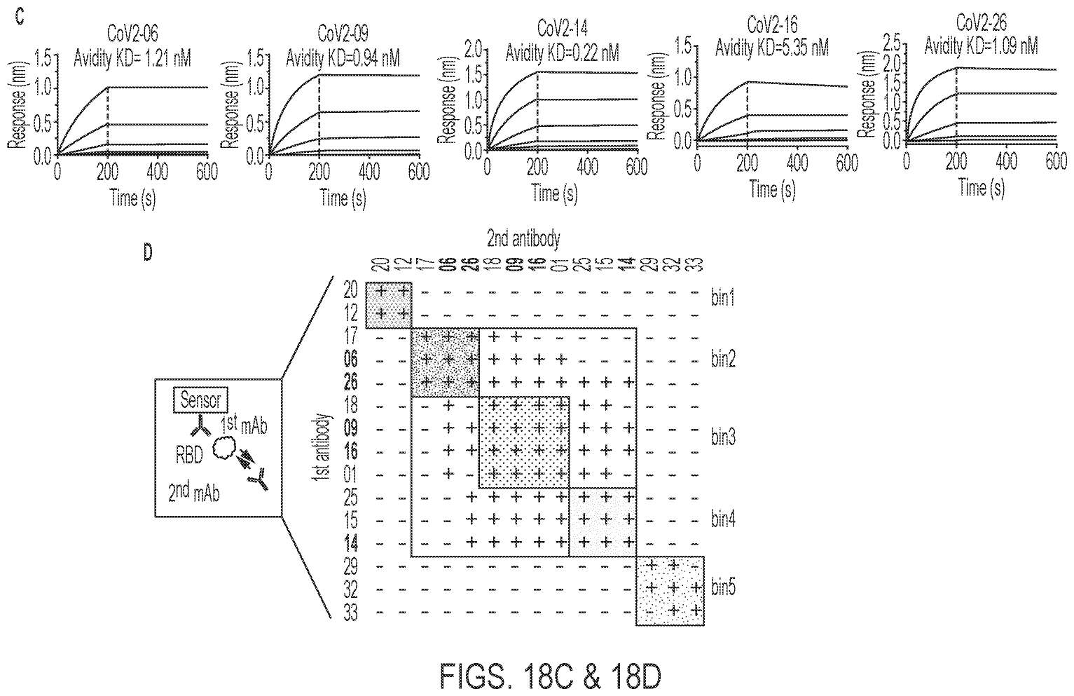

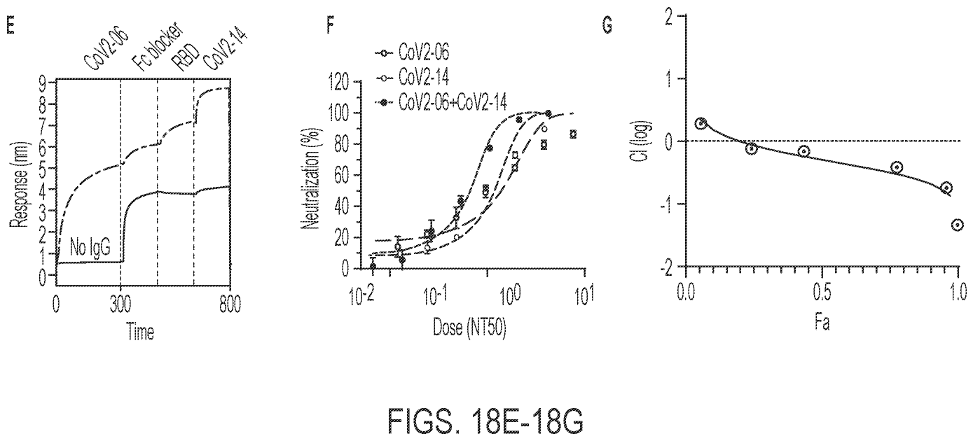

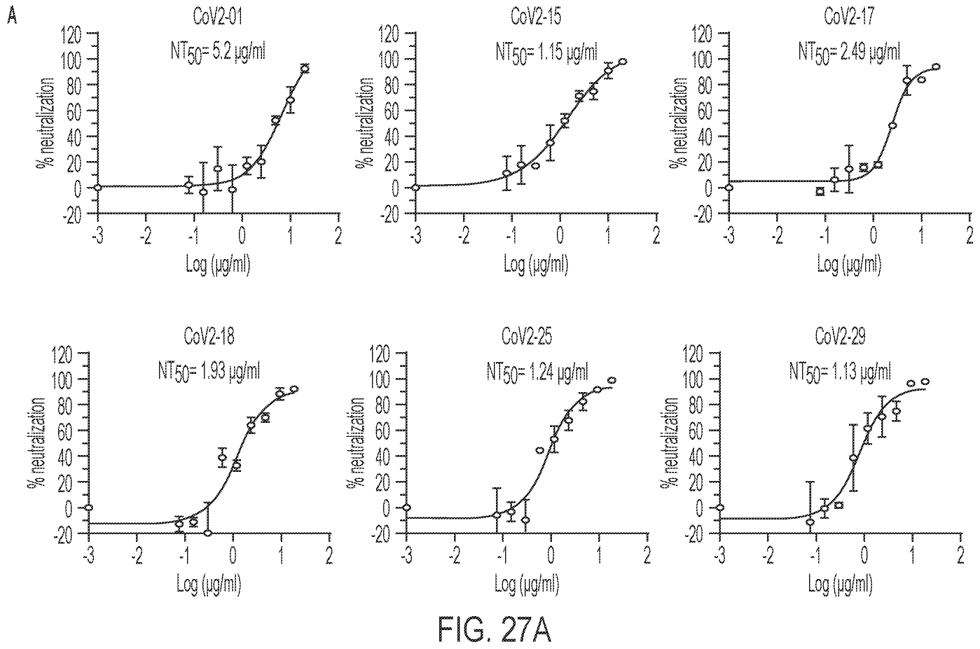

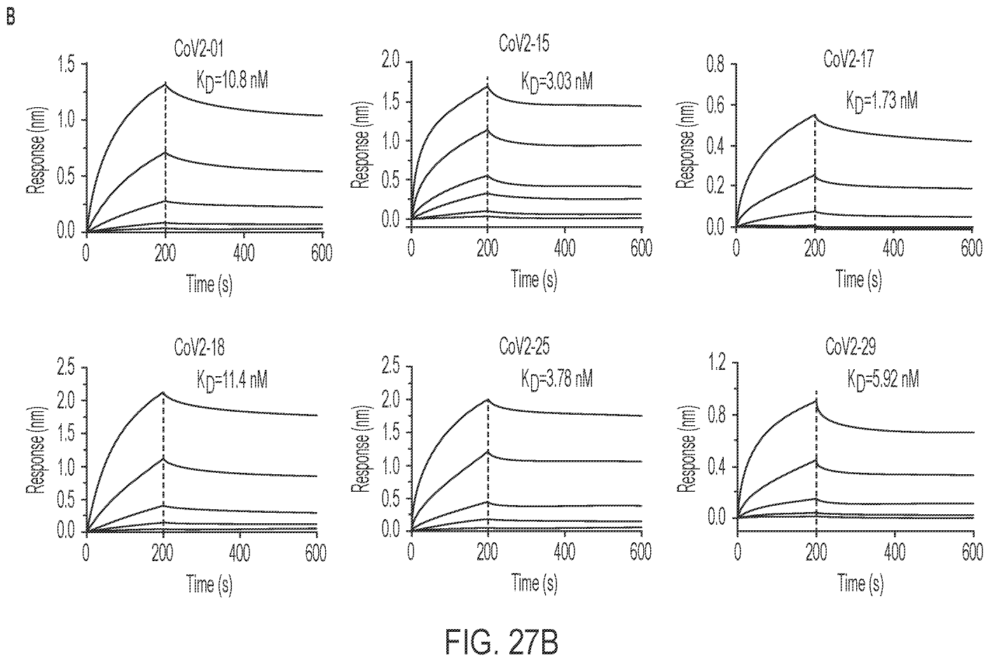

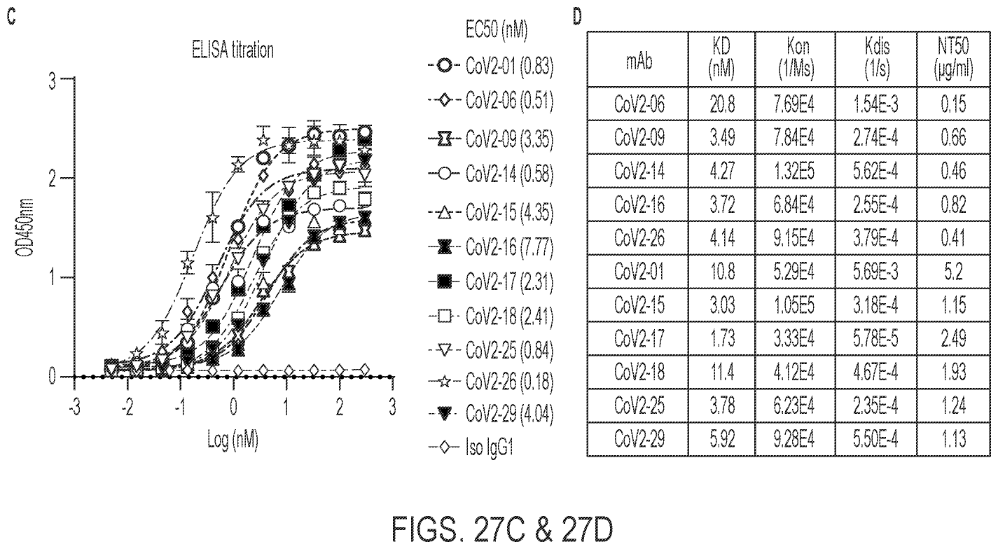

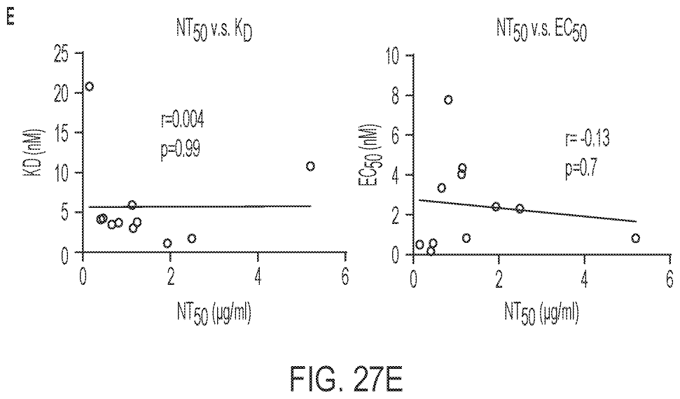

Identification of neutralizing mAbs with simultaneous binding to RBD. The inventors assessed the neutralization potency of the 11 mAbs with NT₇₅<10 μg/ml by a titration assay. Their 50% neutralization titers (NT₅₀s) were between 0.15 and 5.2 μg/ml (FIG. 18A and FIG. 27A). The top five mAbs (CoV2-06, 09, 14, 16, and 26) had NT₅₀ values below 1 μg/ml, with CoV2-06 being the most potent (FIG. 18A). The inventors determined the kinetic binding affinities (K_{D}) of the 11 mAbs to the RBD with a biolayer interferometry (BLI) assay and the equilibrium binding affinities (EC₅₀) with an ELISA titration. The K_{D} values were between 1.73 and 20.8 nM (FIG. 18B and FIG. 27B). The EC₅₀ values were between 0.18 and 7.77 nM (FIGS. 27C-D). The neutralizing activities (NT₅₀) did not correlate with binding affinities to RBD, K_{D} values or EC₅₀ values (FIG. 27E). For the top five mAbs, their apparent affinities (avidities) to the trimeric S protein were between 0.22 and 5.35 nM (FIG. 18C).

After characterizing the binding affinity and neutralizing activity of these mAbs, the inventors sought to identify antibody partners suitable to formulate a cocktail. They selected the 11 potent neutralizing mAbs, together with the two cross-reactive mAbs (CoV2-12 and CoV2-20) and two relatively weak neutralizing mAbs (CoV2-32 and CoV2-33). They performed epitope binning for these selected mAbs, evaluating their ability to compete with each other for binding of RBD. These mAbs delineated five epitope bins, which were designated bin 1 to 5 (FIG. 18D). The top five neutralizing mAbs (CoV2-06, 09, 14, 16, and 26) were in bins 2-4: two in bin 2 (CoV2-06 and 26), two in bin 3 (CoV2-09 and 16), and one in bin 4 (CoV2-14). The two cross-reactive mAbs (CoV2-12 and CoV2-20) were grouped into bin 1. The two weak neutralizing mAbs (CoV2-32 and CoV2-33) and mAb CoV2-29 were grouped into bin 5. Bins 2, 3 and 4 are closely related; antibodies in adjacent bins demonstrated some degree of cross-competition. The inventors selected CoV2-06 (bin2) and CoV2-14 (bin4) for combination studies because they are among the top five neutralizing mAbs and can simultaneously bind to the RBD (FIGS. 18D-E). The cocktail combination of CoV2-06 and CoV2-14 showed a synergy in neutralizing SARS-CoV-2 in vitro (FIGS. 18F-G). These data suggest that CoV2-06 and CoV2-14 are ideal partners to formulate a cocktail.

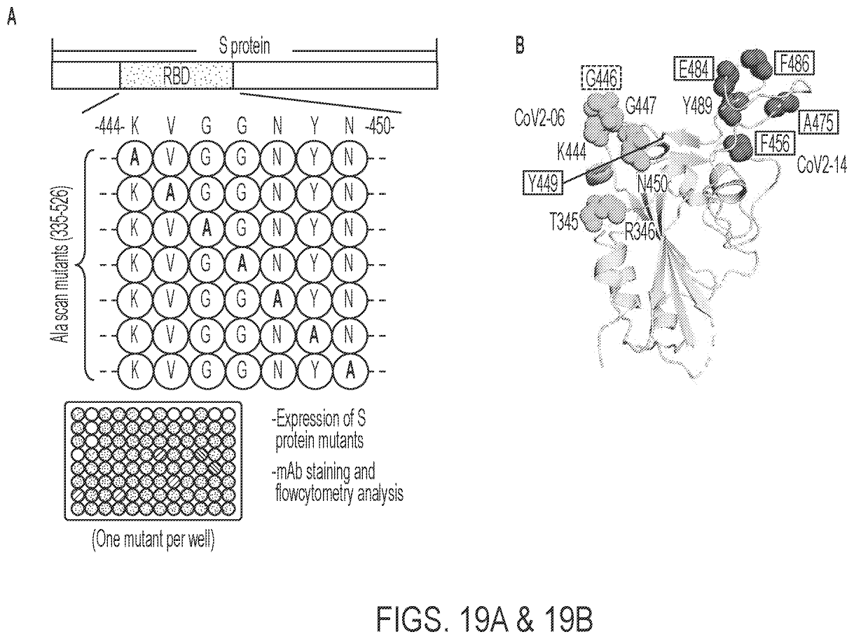

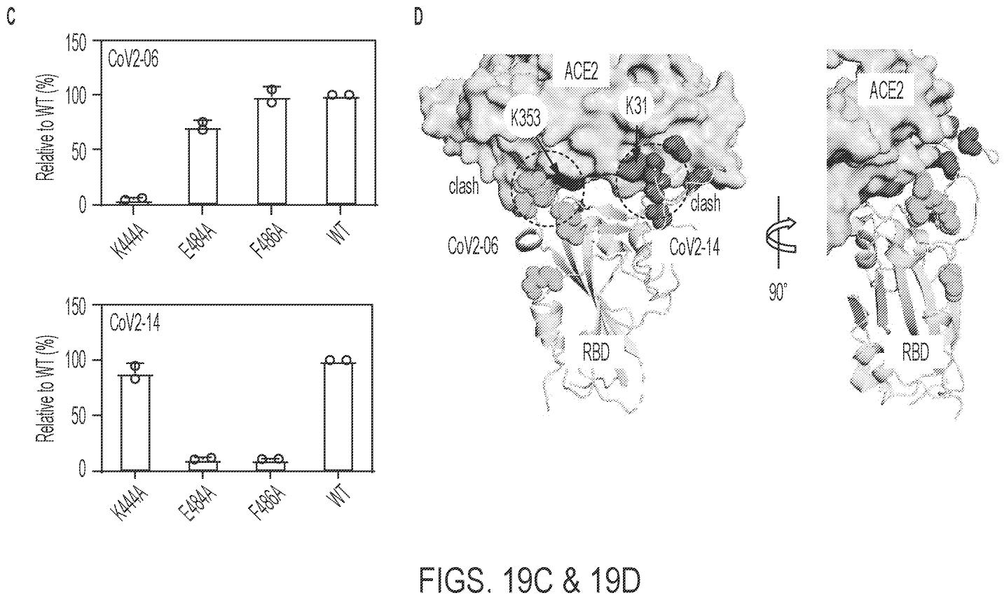

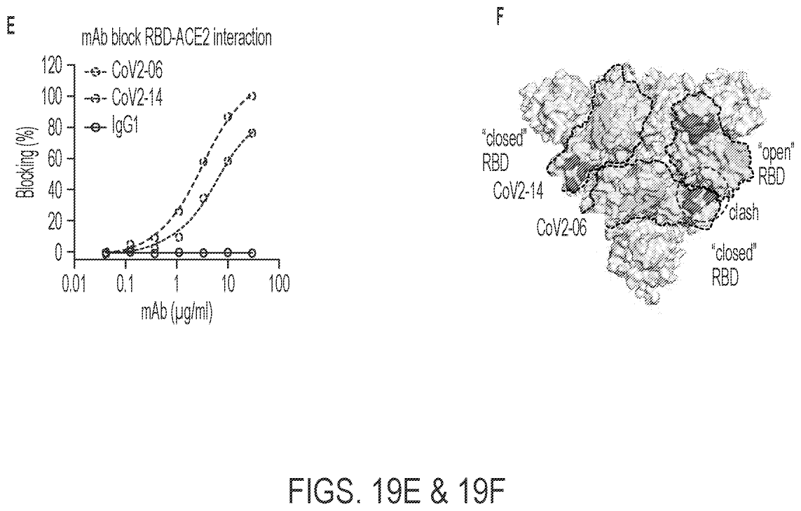

Molecular determinants on the RBD for cocktail mAbs. To define the critical RBD residues for binding of CoV2-06 and CoV2-14, the inventors constructed a comprehensive SARS-CoV-2 RBD mutation library using the full-length protein, in which 184 residues of the RBD (between residues 335-526) were individually mutated to alanine, and alanine residues to serine. Each S protein RBD mutant was individually expressed in HEK293-T cells for determining mAb binding (FIG. 19A). The residues T345, R346, K444, G446, G447, Y449 and N450 of SEQ ID NO: 401 were identified as critical for CoV2-06 binding. The residues F456, A475, E484, F486 and Y489 were identified as critical for CoV2-14 binding (FIG. 19B and Table 6). All of these critical residues except T345 and R346 were in the receptor-binding motif (RBM); six were in direct contact with ACE2 (Slater, H., 2020) (FIG. 19B). Importantly, none of the critical residues for the two mAbs overlapped. In support of the mapping results, in an ELISA analysis of mAb binding to sCoV2-RBD, the K444A mutation abolished the binding of CoV2-06. Likewise, the E484A or F486A mutations abolished the binding of CoV2-14 (FIG. 19C). The K31 and K353 residues in ACE2 are two virus-binding hotspots in the RBD-ACE2 interface (Shang et al., 2020). Interestingly, the CoV2-14 epitope residues E484 and F486 are in direct contact with ACE2 K31 (Wang et al., 2020) and the CoV2-06 epitope is adjacent to the ACE2 K353 (FIG. 19D). The epitope locations indicate that each of the two mAbs target a different hotspot to block ACE2 binding. In agreement with this result, both CoV2-06 and CoV2-14 inhibited sCoV2-RBD binding to ACE2 in a dose-dependent manner (FIG. 19E). The inventors also visualized the epitope locations on the trimeric spike protein to analyze their antibody accessibility. On the trimeric spike protein, the CoV2-06 epitope is accessible in both the “open” and “closed” RBD. In contrast, the CoV2-14 epitope is more accessible in the “open” RBD than the “closed” RBD, especially if the “closed” RBD is adjacent to an “open” RBD (FIG. 19F). Antibodies that target both the more accessible and less accessible sites can have high potency (Ju et al., 2020; Yuan et al., 2020a).

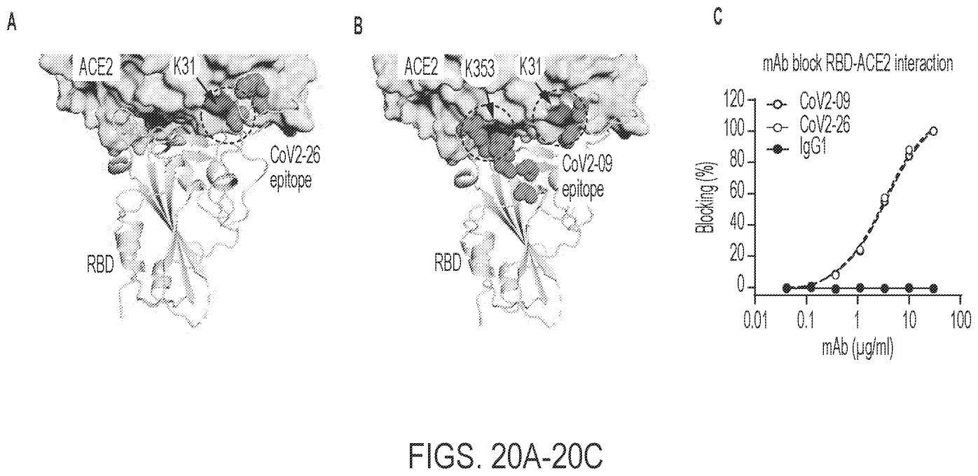

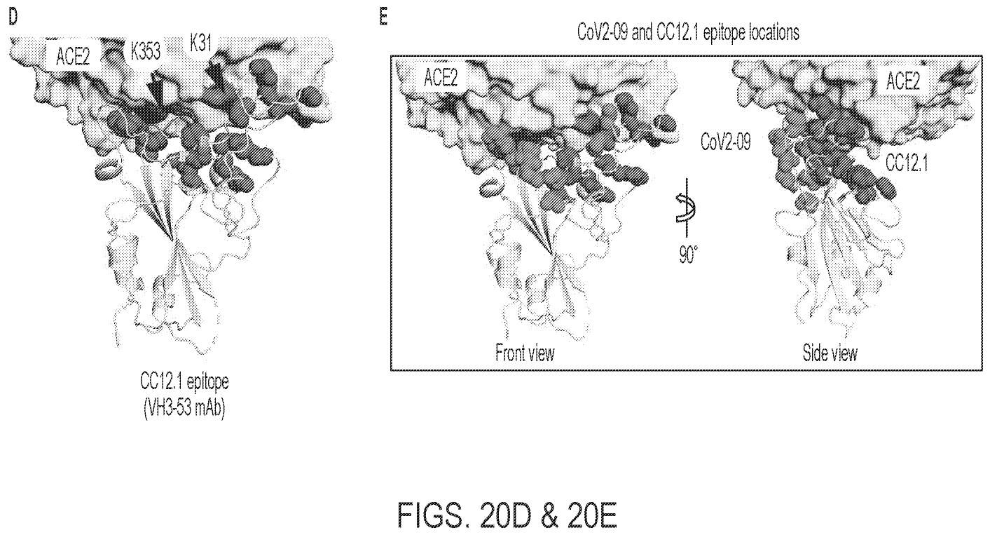

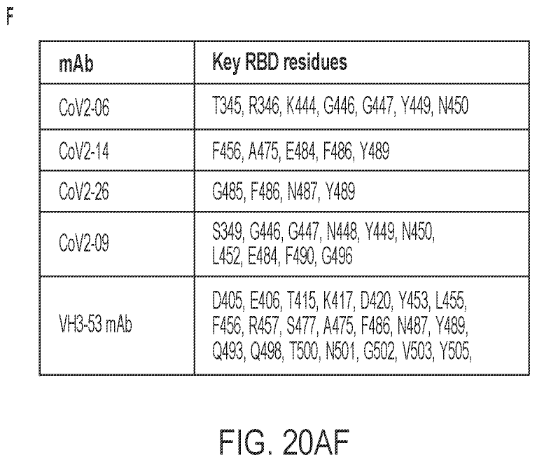

The inventors also mapped the epitopes of CoV2-09, CoV2-16, and CoV2-26 from the top five potent neutralizing mAbs (FIGS. 20A-F and Table 6). Interestingly, although CoV2-14 and CoV2-26 bind to similar RBD epitope and shared two critical residues (FIG. 20F), only CoV2-14 could bind to RBD simultaneously with CoV2-06 (FIG. 18D). This suggests that CoV2-26 has a different approaching angle that is not compatible with simultaneous binding by CoV2-06. Thus, the inventors understand that both non-overlapping epitopes and lack of competition for binding are critical determinants for selecting cocktail mAbs. CoV2-09 and CoV2-16 have the same critical residues (Table 6), possibly because they share the same heavy chain (FIG. 4). Therefore, the inventors focused on CoV2-09 for further analysis. The binding epitopes of CoV2-09 and CoV2-26 suggest that they are also ACE2-competing mAbs (FIGS. 20A-B). This was validated in a BLI-based competition assay (FIG. 20C). Unlike CoV2-06, CoV2-14 or CoV2-26 mAbs, CoV2-09 had an epitope that is adjacent to both the K353 and K31 hotspots in ACE2 (FIG. 20B), which was similar to the VH3-53 like antibodies (Yuan et al., 2020a) (FIG. 20D). However, the epitopes of CoV2-09 and VH3-53 mAbs were distinct from each other with no overlapping residues (FIG. 20E-F). A number of VH3-53 like antibodies with potent neutralizing activities have been isolated and their epitopes are defined (Yuan et al., 2020a). Due to the promising nature of these antibodies, future studies shall determine whether CoV2-09 and VH3-53 like mAbs can simultaneously bind to RBD and to form an effective cocktail. Collectively, these data elucidated the molecular basis for CoV2-06 and CoV2-14 as effective cocktail mAbs, and also identified other determinants potentially suitable for designing mAb cocktails.

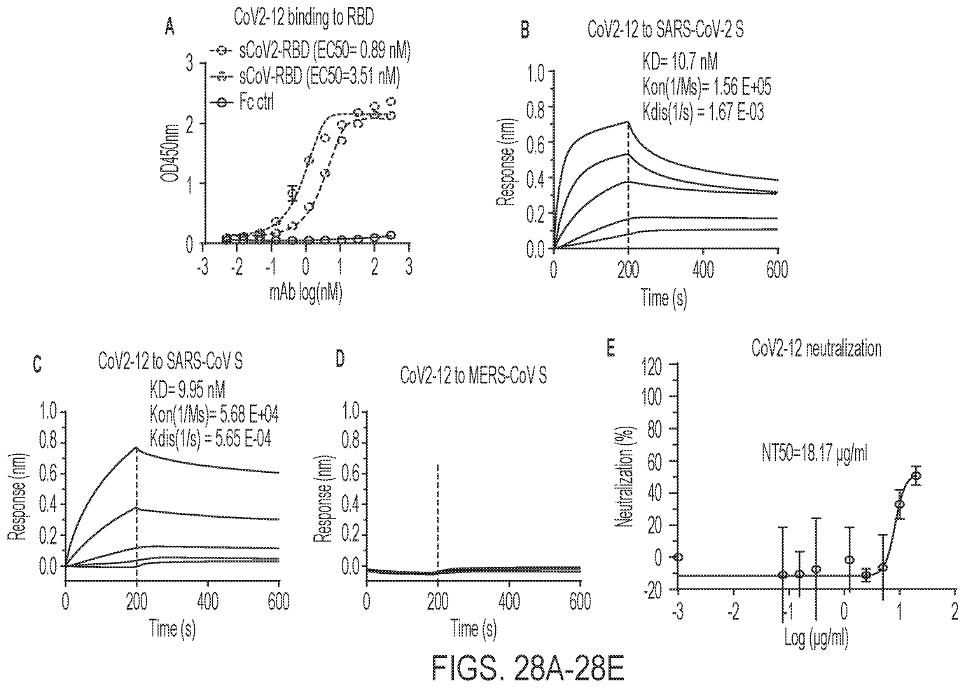

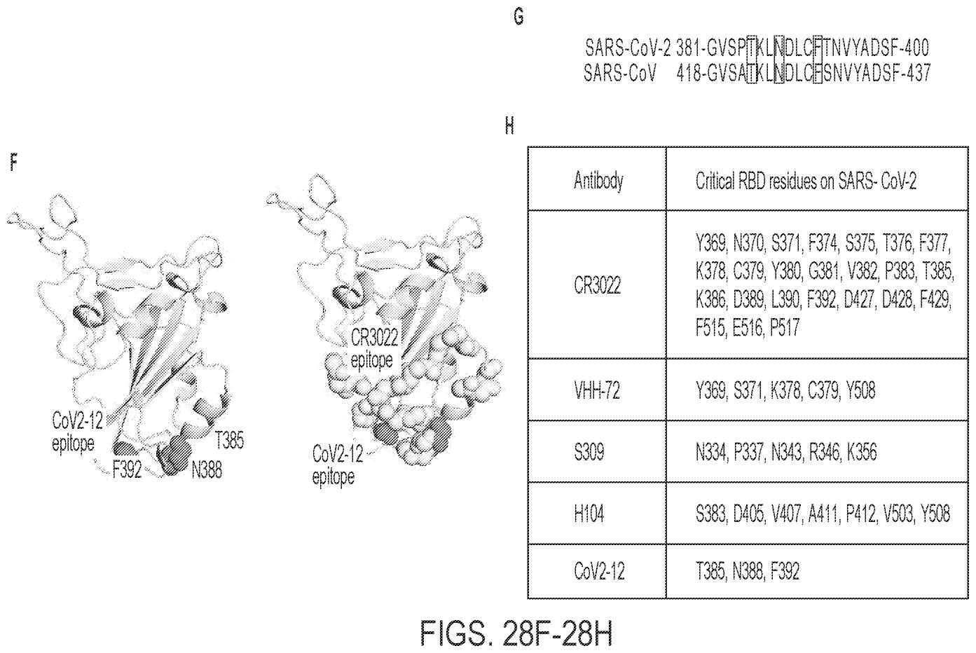

CoV2-12 is a rare mAb that cross-reacts with SARS-CoV-2 and SARS-CoV. The inventors further characterized its binding, neutralization, and epitope (FIGS. 28A-H and Table 6). CoV2-12 binds to the RBD proteins of the SARS-CoV-2 and SARS-CoV with comparable EC₅₀ values (FIG. 28A). It also binds to the S proteins of the SARS-CoV-2 and SARS-CoV, but not the MERS-CoV, with comparable avidities (FIGS. 28B-D). CoV2-12 neutralizes the SARS-CoV-2 with an NT 50 of 18.17 μg/ml (FIG. 28E). These results indicate that CoV2-12 targets a conserved neutralizing epitope. Epitope mapping indicated that CoV2-12 binds an RBD site distal from the RBM (FIG. 28F). This result is consistent with its ability to simultaneously bind to RBD with the top five neutralizing mAbs (FIG. 18D). The critical residues identified for CoV2-12 (corresponding to T385, N388, and F392 of SEQ ID NO: 401 and T372, N375, and F379 of SEQ ID NO: 402) are completely conserved between SARS-CoV-2 and SARS-CoV (FIG. 10A and FIG. 28G). Two residues (T385 and F392 of SEQ ID NO: 401) overlapped with the contact residues of the cross-reactive mAb CR3022 (Yuan et al., 2020b), but no residues overlapped with several other reported cross-reactive mAbs, including VHH-72 (Wrapp et al., 2020), 5309 (Pinto et al., 2020), and H104 (Lv et al., 2020). The N388 was a critical residue unique to CoV2-12 (FIGS. 28F and 28H). Although the epitopes of CoV2-12 and CR3022 partially overlapped, CoV2-12 exhibited 50% neutralization at 18.17 μg/ml while CR3022 exhibited no neutralization against SARS-CoV-2 at 400 μg/ml (Yuan et al., 2020b). The difference in critical contact residues could be one reason these two mAbs behave differently in neutralizing SARS-CoV-2.

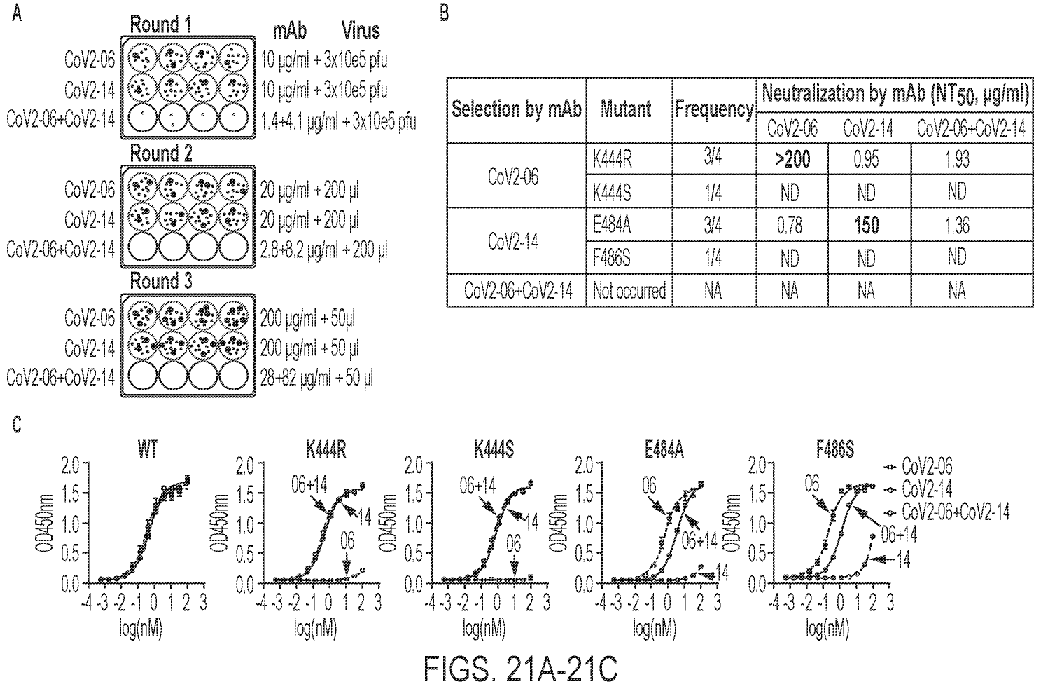

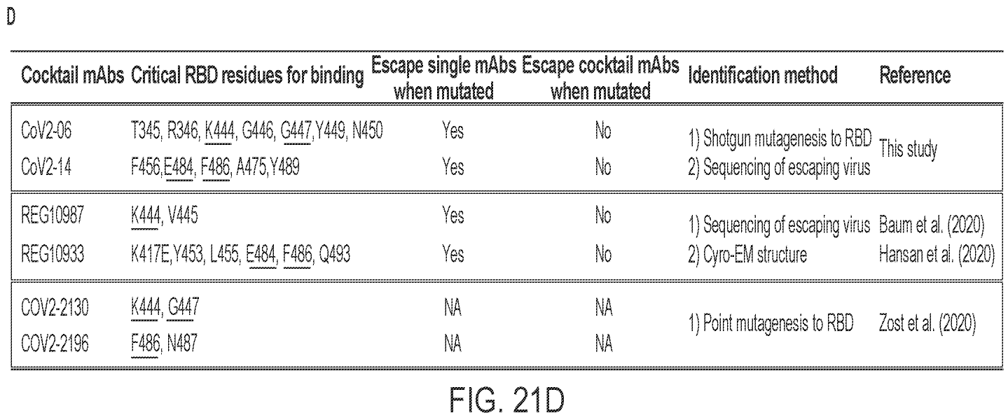

CoV2-06 and CoV2-14 cocktail prevents neutralization escape of live SARS-CoV-2. The inventors used the authentic live SARS-CoV-2 to evaluate neutralization escape. They passaged the SARS-CoV-2-mNG virus in the presence of CoV2-06, CoV2-14, CoV2-06+CoV2-14 for three rounds (FIG. 21A). The inventors could recover virus in the presence of individual CoV2-06 or CoV2-14 mAbs but not in the presence of the cocktail mAbs (FIG. 21A). They then sequenced the S region of the viruses recovered from the four replicative selections to identify escape mutations. Under CoV2-06 selection, three independently selected viruses had a K444R mutation (all mutations corresponding to positions in SEQ ID NO: 401) and one selected virus had a K444S mutation. Under CoV2-14 selection, three independently selected viruses had an E484A mutation and one selected virus had an F486S mutation (FIG. 21B). Outside the RBD, additional mutations in the N-terminal domain (NTD) of S were also observed in some selected viruses. For example, H66R or H66R+R190K were observed under CoV2-06 selection, and N74K was observed under CoV2-06 or CoV2-14 selection. The inventors sought to confirm whether the mutations in the RBD but not the NTD are responsible for resistance. They focused on the most frequent K444R and E484A mutations and constructed two recombinant SARS-CoV-2 viruses with point mutation K444R or E484A. The two mutant viruses were then analyzed for neutralization by individual CoV2-06, CoV2-14 mAbs, and the CoV2-06+CoV2-14 cocktail. The K444R mutant virus could escape the neutralization by CoV2-06 but not CoV2-14; the E484A mutant virus could escape the neutralization by CoV2-14 but not neutralization by CoV2-06 (FIG. 21B). The mAb cocktail maintained neutralization against both the K444R and E484A mutant viruses (FIG. 21B). These results demonstrated that the mAb cocktail of CoV2-06 and CoV2-14 is effective in preventing SARS-CoV-2 escape mutations in vitro.

Next, the inventors sought to further investigate whether the loss of neutralization of single mAbs to the mutant viruses is the result of diminished RBD binding activities. Toward this end, the inventors generated four sCoV2-RBD proteins with individual mutations of K444R, K444S, E484A, or F486S. While CoV2-06 had almost no binding to sCoV2-RBD proteins with K444R or K444S mutations, it maintained binding to sCoV2-RBD proteins with E484A and F486S mutations (FIG. 21C). Similarly, CoV2-14 lost binding to sCoV2-RBD proteins with E484A or F486S mutations but maintained binding to sCoV2-RBD proteins with K444R or E484A mutations (FIG. 21C). The mAb cocktail maintained binding to all the sCoV2-RBD mutant proteins (FIG. 21C). These binding data are in agreement with the epitope mapping findings that CoV2-06 and CoV2-14 target two non-overlapping epitopes. These results indicate that mutations on a single antigenic site do not abolish the binding and neutralization of CoV2-06+CoV2-14 at the same time, which can occur for cocktail mAbs with overlapping epitopes (Baum et al., 2020).

Three independent studies, including the inventors', have identified and characterized cocktail mAbs targeting the RBD (Zost et al., 2020b, Hansen et al., 2020; Baum et al., 2020). FIG. 21D summarized the RBD residues critical for the cocktail mAbs and escape mutations. Interestingly, mAbs CoV2-06, REGN10987 (imdevimab), and COV2-2130 (cilgavimab) shared the same critical residue K444; mAbs CoV2-14, REGN10933 (casirivimab), and COV2-2196 (tixagevimab) shared the same critical residue F486. Other shared residues, such as G447 for CoV2-06 and COV2-2130, E484 for CoV2-14 and REGN10933, were also observed (FIG. 21D). Both CoV2-06+CoV2-14 and REGN10987+REGN10933 cocktails were able to prevent viral escape while individual mAbs were not (FIG. 21D). The critical residues on the RBD for these analogous mAb cocktails are key determinants for formulating optimal mAb cocktails against SARS-CoV-2.

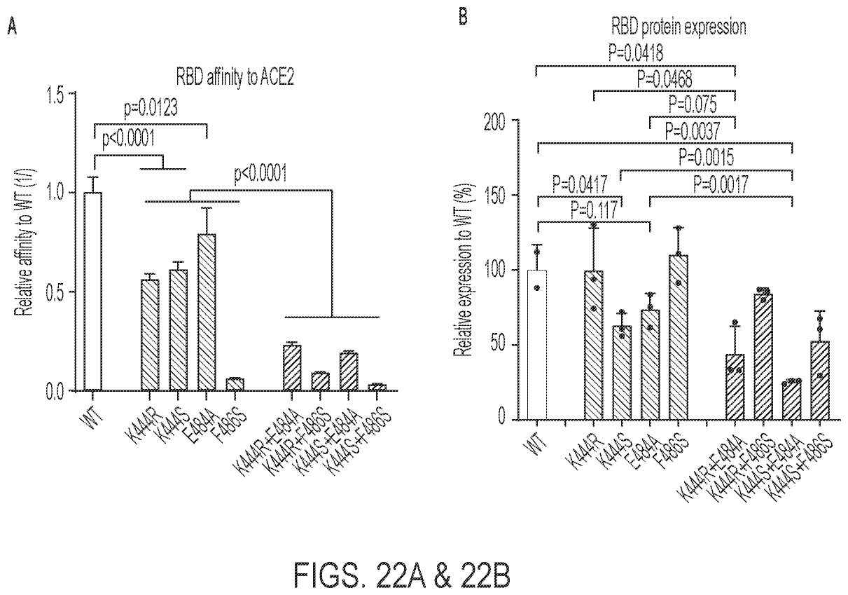

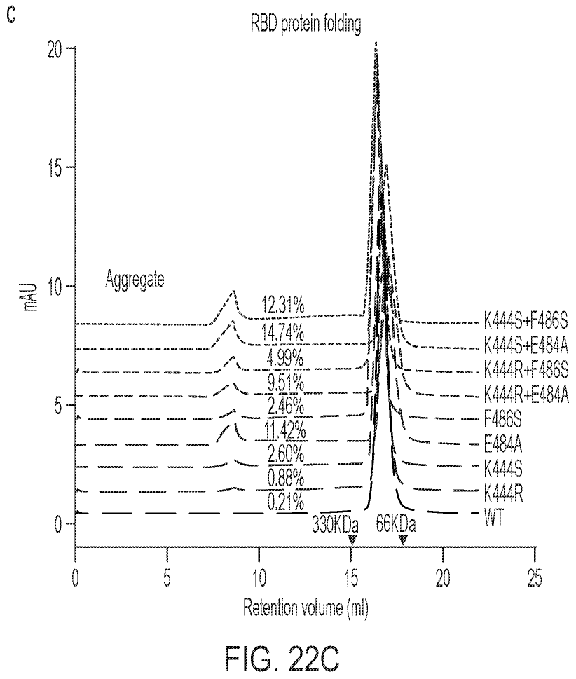

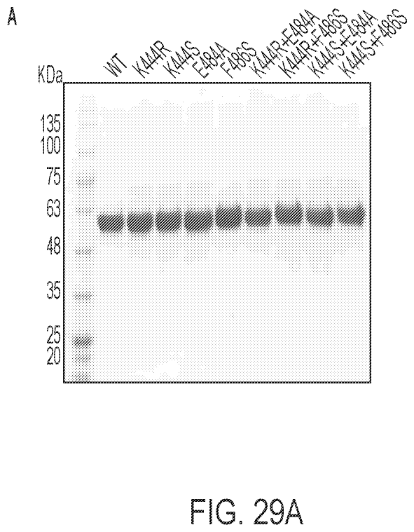

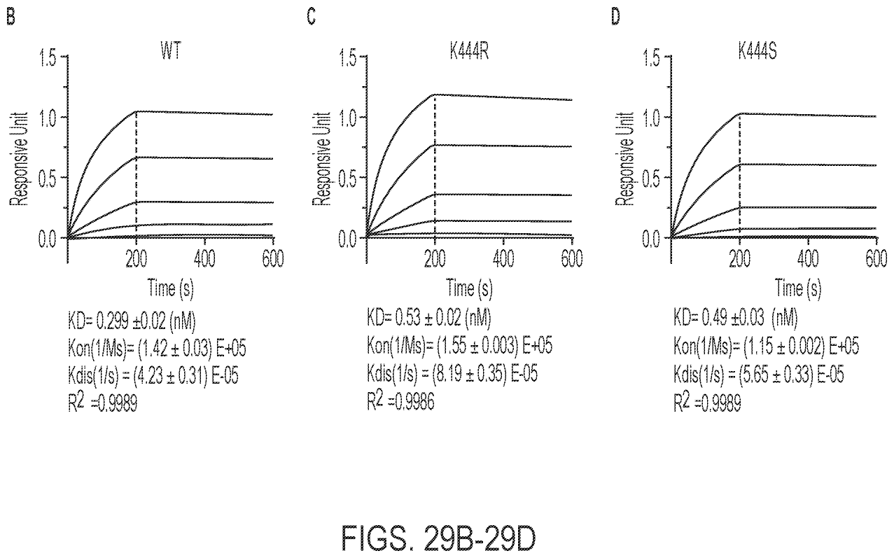

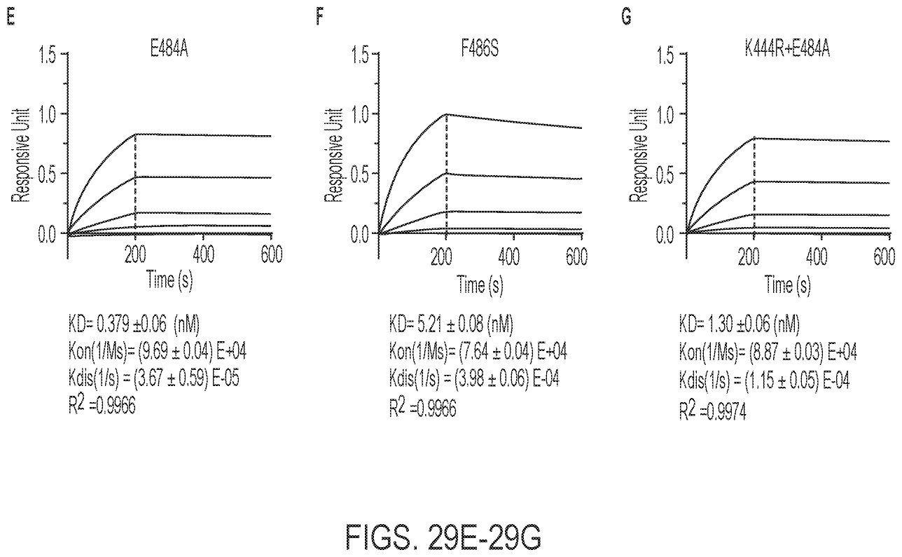

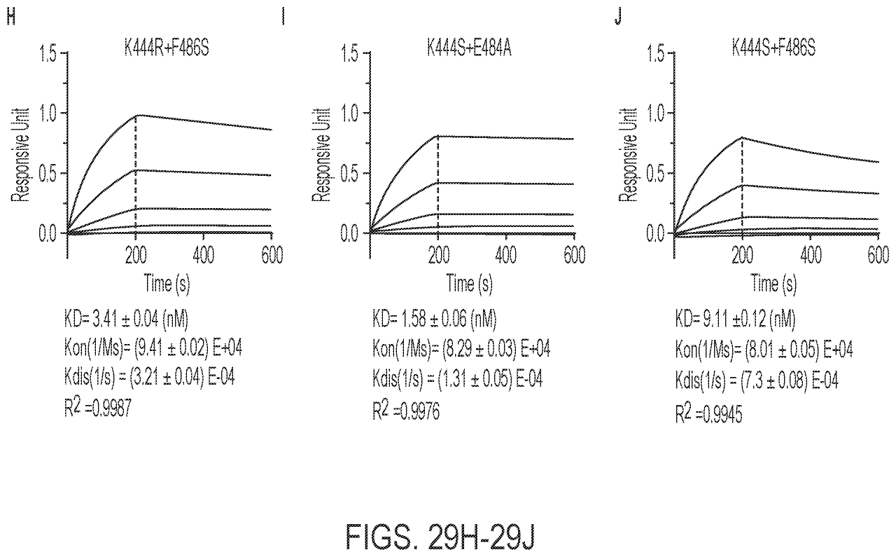

CoV2-06 and CoV2-14 cocktail imposes a strong mutational constraint on the RBD. While some RBD mutations are well tolerated, other mutations are deleterious for RBD function and therefore constrained in SARS-CoV-2 (Starr et al., 2020). The inventors reasoned that simultaneous mutations on the two distinct binding sites of CoV2-06 and CoV2-14, which are required for virus to escape neutralization by the cocktail, would be more constrained than mutations on the binding sites of individual mAbs. To test this hypothesis, they generated eight sCoV2-RBD mutant proteins, four with individual mutations of single binding sites (K444R, K444S, E484A and F486S) and four with simultaneous mutations of both binding sites (K444R+E484A, K444R+F486S, K444S+E484A and K444S+F486S) (FIG. 29A). These single-site or double-site RBD mutants were analyzed for their affinity to ACE2 (FIG. 22A and FIGS. 29B-J), protein expression (FIG. 22B) and folding stability (FIG. 22C). Single-site mutations of K444R, K444S, E484A, and F486S reduced the sCoV2RBD/ACE2 binding affinities to 56%, 61%, 79% and 6% of the WT, respectively. In comparison, double-site mutations of K444R+E484A, K444R+F486A, K444S+E484A, and K444S+F486S further reduced the sCoV2-RBD/ACE2 binding affinities to 23%, 9%, 19% and 3% of the WT, respectively (FIG. 22A and FIGS. 29B-J). Similarly, while single-site mutations altered the RBD expression to 69-110.1% of the WT, double-site mutations reduced the expression to 25.5-84.2% of the WT (FIG. 22B). The size exclusion chromatography (SEC) analysis showed protein aggregates of 0.88-11.42% and 4.99-14.74% for RBD with single-site mutations and double-site mutations, as compared to only 0.21% of aggregates for wild-type RBD (FIG. 22C). These data indicate that double mutations at both the CoV2-06 and CoV2-14 epitope sites attenuated the receptor binding affinity and stability of the RBD more than that of the single-site mutations, suggesting that such double-site mutations would have deleterious effects on viral fitness.

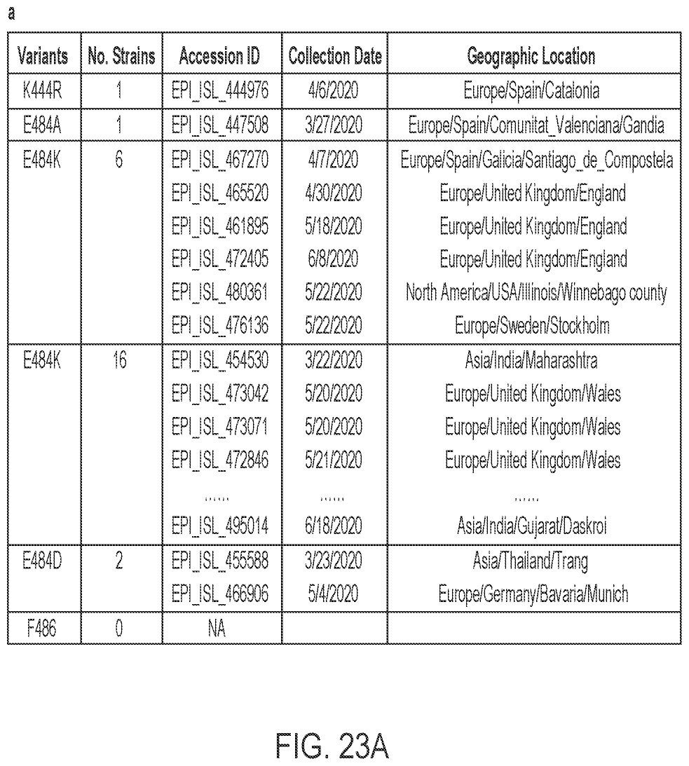

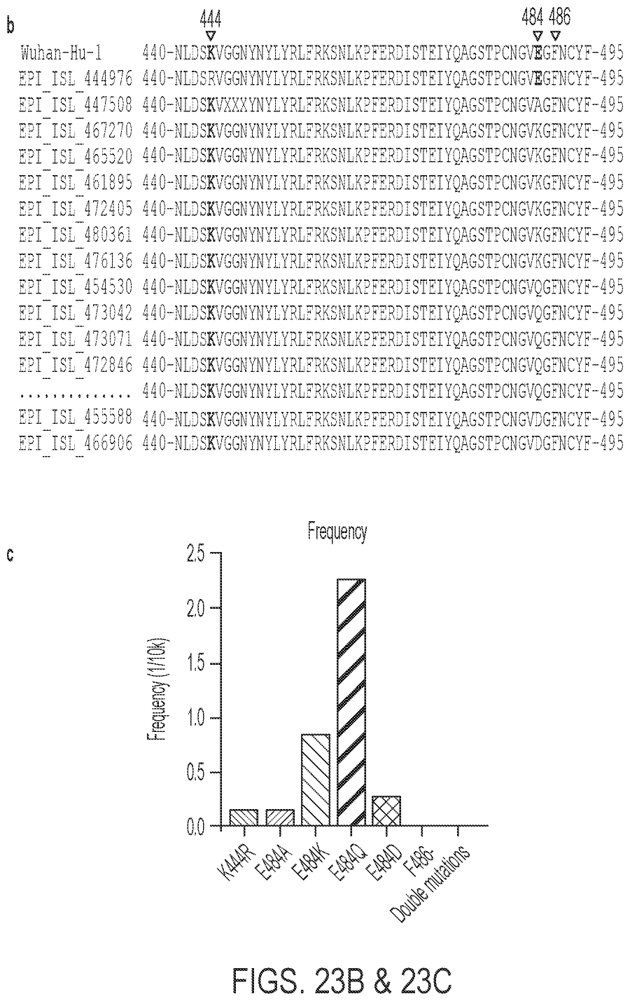

No evidence for the occurrence of virus variants with double-site mutation. To gain insights of RBD mutations in naturally occurred virus variants during the global transmission, the inventors analyzed 70,934 publicly available viral genome sequences (as of Jul. 23, 2020) (FIGS. 23A-C). Although the frequency was very low, 26 clinical isolates with RBD polymorphisms analogue to the mAb-escaping mutants were identified. Among these isolates, one had the K444R mutation, one had the E484A mutation, six had the E484K mutation, sixteen had the E484Q mutation and two had the E484D mutation. No polymorphism at F486 was found (FIG. 23A). It is likely that these virus variants occurred as a result of selection by the epitope-directed neutralizing antibodies in COVID-19 patients (Zost et al., 2020b; Baum et al., 2020). However, the low frequency of occurrence indicates that these virus variants may have compromised epidemiologic fitness during transmission. More importantly, alignment of the RBD regions of these 26 isolates demonstrated no occurrence of virus variants with simultaneous mutations on the K444 and E484 sites, or the K444 and F486 sites, to the date of analysis (FIGS. 23B-C). These results suggest that virus variants with simultaneous mutations at both CoV2-06 and CoV2-14 epitopes either have not occurred or occurred at an extremely low frequency that is beyond epidemiologic monitoring.

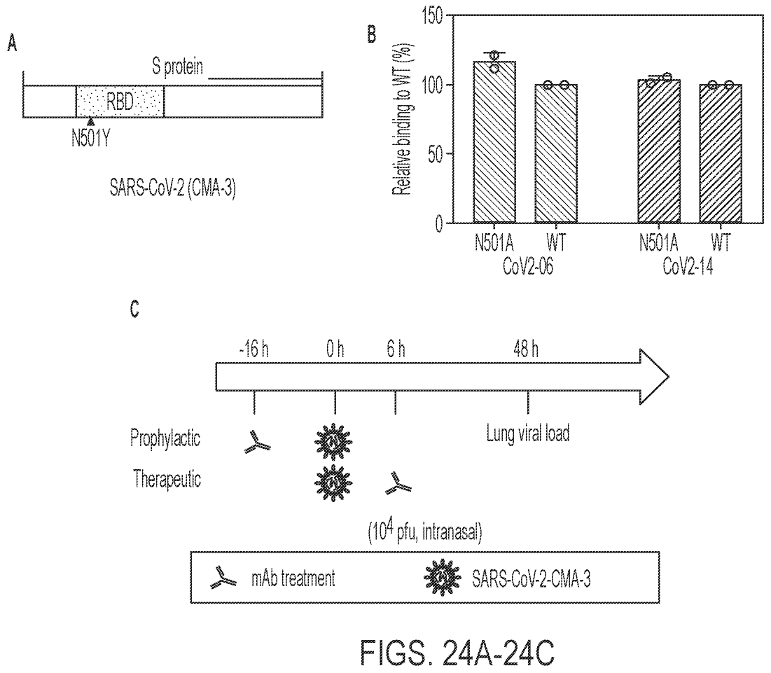

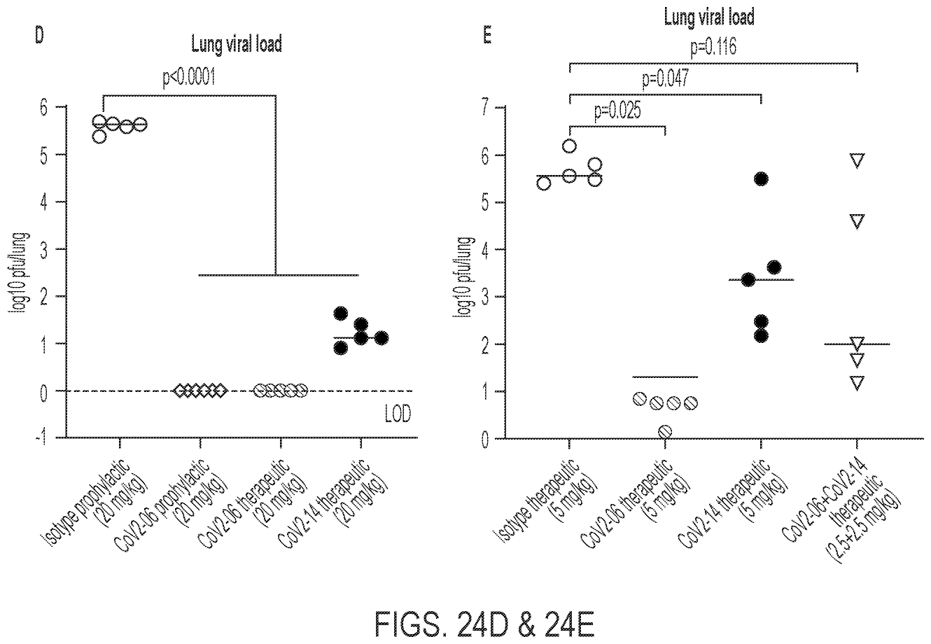

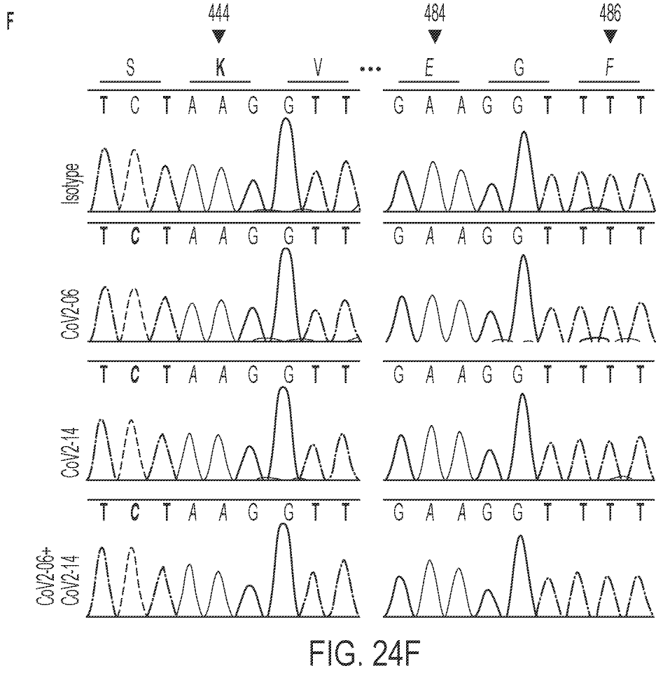

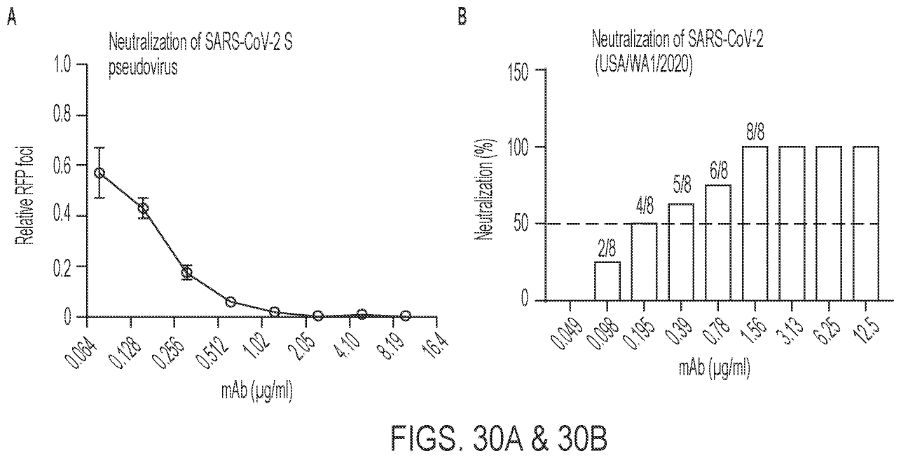

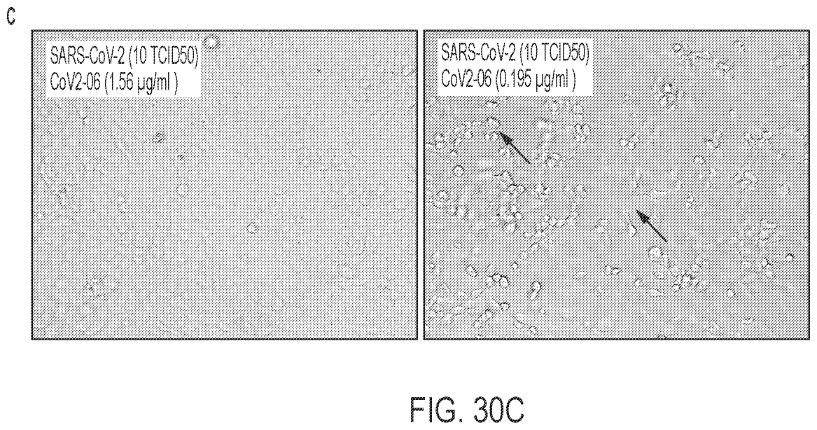

Antibody protection against SARS-CoV-2 infection in mice. The inventors evaluated the protective effects of CoV2-06 and CoV2-14 individually and in combination in a mouse model of infection with a mouse-adapted virus strain (CMA-3). This virus has a N501Y adaptive mutation in the RBD of the S region to facilitate mouse infection (FIG. 24A). Utilization of mouse-adapted virus in evaluation of vaccine efficacy and antibody protection in mice had been demonstrated elsewhere (Zhang et al., 2020; Dinnon et al., 2020). The N501 was not a critical RBD residue for CoV2-06 or CoV2-14. The inventors confirmed that mutation of N501 did not reduce RBD binding by the two antibodies (FIG. 24B). The CoV2-06 mAb is the most potent neutralizing mAb in this study. Its neutralization activity was independently validated by using SARS-CoV-2 S pseudovirus (FIG. 30A) and the SARS-CoV-2 clinical isolate (USA/WA1/2020) (FIGS. 30B-C). To evaluate antibody protection in vivo, mice were given an intraperitoneal injection of CoV2-06 at 16 hours before or 6 hours after intranasal challenge with 10⁴ plaque-forming unit (pfu) of the CMA-3 virus. Two days-post infection, the lung tissues were harvested, and the viral loads were measured (FIG. 24C). For both the prophylactic and therapeutic treatments, CoV2-06 at 20 mg/kg reduced lung viral load to undetectable level. Therapeutic treatment with CoV2-14 at 20 mg/kg reduced lung viral load by 4˜5 log 10 fold (FIG. 24D). The inventors further demonstrated that therapeutic treatments with CoV2-06 and CoV2-14 individually and in combination conferred protection in the same model. The cocktail was less effective than CoV2-06 alone possibly due to the lower dose (5 mg/kg) used (FIG. 24E). Treatment with the sub-optimal dose allow virus harvesting in the lung for determination whether resistant mutations occurred. For both CoV2-06 and CoV2-14 monotherapy groups and the cocktail antibody treatment, no mutation on key epitope residues was observed (FIG. 24F). These data demonstrate effective antibody protections against SARS-CoV-2 in mice.

### Example 5—Discussion

The inventors identified the molecular determinants on the RBD that are optimal for selecting effective mAb cocktails against SARS-CoV-2. They also revealed the mechanism by which a mAb cocktail prevents escape mutations using live SARS-CoV-2 in cell culture. A mAb cocktail (REG10987+REG10933) has entered phase 2/3 clinical trials (NCT04425629, NCT04452318). Using the VS V-SARS-CoV-2 S recombinant virus, neutralization escape had been evaluated for the REG10987+REG10933 cocktail (Ko et al., 2018). Interestingly, the inventors independently identified a mAb cocktail (CoV2-06+CoV2-14) that shares similar binding epitopes with the REG10987+REG10933 cocktail. As VSV and SARS-CoV-2 are different, the inventors used the authentic SARS-CoV-2 to evaluate neutralization escape. Although different viral systems were used, both studies demonstrated that only the mAb cocktail, not individual mAbs, can prevent escape mutations (Baum et al., 2020). Indeed, SARS-CoV-2 escapes from individual mAb inhibition rapidly, within 2-3 passages, regardless of mAb neutralization potency (Baum et al., 2020) Amino acid K444 is a critical RBD residue for both CoV2-06 and REG10987. Amino acids E484 and F486 are critical RBD residues for both CoV2-14 and REG10933. Functional analysis validated that mutations of these residues are responsible for viral escape from the individual mAbs Amino acid K444 is also a critical epitope residue for other SARS-CoV-2 neutralizing mAbs, including P2B-2F6 and 5309 (Ju et al., 2020; Pinto et al., 2020); E484 is a critical residue for P2B-2F6 (Ju et al., 2020); and F486 is a critical residue for VH3-53 like mAbs (Yuan et al., 2020a). The inventors show that single-site mutations of these residues compromised the RBD on its affinity for ACE2 and the folding stability slightly, but double-site mutations attenuated the fitness of RBD dramatically. These results are consistent with evidence showing no natural occurrence of virus variants with simultaneous mutations of the binding residues for the two mAbs. The inventors' findings provide mechanistic insights into how such cocktails prevent viral escape. It is important in future studies to evaluate whether antibody cocktails can prevent viral escape in vivo. In this particular experiment, the inventors did not see resistance-related mutation in single antibody or cocktail antibody treated mice. This perhaps because the frequency of mutant virus, if any emerged, was extremely low. Nevertheless, they need to mention that virus escape of neutralizing antibodies may be different in mouse and human systems. Monitoring the dynamic changes of these key mutation sites in clinical studies of antibody monotherapy or cocktail therapy and characterizing their impact on viral pathogenesis will fill this important knowledge gap.

To date, three studies, including ours, identified anti-SARS-CoV-2 mAb cocktails and characterized their epitopes RBD (Zost et al., 2020b, Hansen et al., 2020; Baum et al., 2020). Surprisingly, these studies independently discovered cocktail mAbs that have similar epitope combinations and share several key amino acid residues. Because the two epitopes are located on different RBM ridges that are well separated (FIG. 19B) (Shang et al., 2020), they are more likely to accommodate two mAbs simultaneously. Binding on non-overlapping epitopes does not necessarily allow simultaneous binding of two mAbs, which is the case for CoV2-26 and CoV2-06. Although CoV2-26 binds to similar epitope and shares key residues with CoV2-14, it is a less than ideal partner than CoV2-14 for combination with CoV2-06 due to the RBD binding competition. This suggests that favorable approaching angles are also important for optimal mAb cocktails. Further structural characterization of the RBD in complex with these mAbs will provide additional insights to understand optimal mAb cocktails. Epitope combinations other than that of CoV2-06 and CoV2-14 might also be attractive to select mAb cocktails. Using CoV2-09, the inventors defined a previously unreported neutralizing epitope. Interestingly, this epitope partially overlapped with epitopes of CoV2-06 and CoV2-14 but not with the epitope of VH3-53 like antibodies (FIG. 20f). Indeed, the epitopes of CoV2-09 and VH3-53 like antibodies are located at two different RBD patches that comprise the ACE2 interface and do not share binding residues (FIGS. 20e-f). These epitopes may permit simultaneous binding of the RBD by CoV2-09 and VH3-53 like antibodies. A number of VH3-53 like antibodies have been evaluated as monotherapies (Rogers et al., 2020; Wu et al., 2020). However, it is not known if they can prevent viral escape. Therefore, future efforts may focus on validating the combination of CoV2-09 and VH3-53 like mAbs as cocktails to prevent viral escape.

In addition to identifying cocktail mAbs and determining their epitopes, the inventors identified a SARS-CoV-2 neutralizing mAb (CoV2-12) with cross-reactivity to SARS-CoV. Epitope mapping identified three critical RBD residues, which are not overlapped with the epitopes of a number of reported cross-reactive mAbs, including VHH-72, 5309, and H104 (Wrapp et al., 2020; Pinto et al., 2020; Lv et al., 2020). Two of the CoV2-12 epitope residues overlap the epitope of CR3022. However, only CoV2-12 exhibits neutralizing activity against SARS-CoV-2. This suggest that other non-overlapped residues, such as N388, might be important for CoV2-12 to exhibit neutralization. Since the epitope residues of CoV2-12 are located in the RBD core region where amino acid mutations are usually deleterious for RBD expression and folding (Gu et al., 2020), certain key epitope residues for CoV2-12 might not be revealed by the RBD mutation library approach that the inventors used. Further structural analysis will provide additional information on this conserved epitope. By focusing on this epitope, future efforts of antibody selection may generate potent and broadly neutralizing mAbs against SARS-CoV-2 and SARS-CoV.

The neutralizing epitopes identified in this study will also be useful for assessing vaccine elicited antibody responses. To avoid potential escape mutations, it is critical for the vaccines to elicit neutralizing antibodies targeting diverse epitopes. Indeed, antibody responses to the two epitopes of CoV2-06 and CoV2-14 are subdominant in some subjects (Zost et al., 2020b), while antibody response to the VH3-53 like antibody epitope is shared in many subjects (Yuan et al., 2020a). In addition, the knowledge on these two epitopes can facilitate the design of vaccines that can elicit dominant antibody responses to these epitopes so that viral escape mutations can be reduced. The knowledge on CoV2-12 epitope is useful for the design of vaccines with potential to elicit more broadly neutralizing antibodies.

In summary, the inventors report the molecular determinants and mechanism for a mAb cocktail that prevents SARS-CoV-2 viral escape mutations. They also identified an epitope combination potentially suitable for the design of other cocktail mAbs, as well as a conserved epitope for selecting cross-reactive neutralizing mAbs. This study is informative for the evaluation of the clinical-stage cocktail mAbs, benefits further selection of other cocktail antibodies against SARS-CoV-2, and aids the assessment of vaccines. Finally, the mAbs the inventors isolated hold promise for further development as antibody therapies for COVID-19.

TABLE 6
Residues critical for mAb binding to SARS-COV-2 S protein
RBD (numbering relative to SEQ ID NO: 401)

| | CoV2- | CoV2- | CoV2- | CoV2- | CoV2- | CoV2- |
|---|---|---|---|---|---|---|
| Mutation | 06 | 14 | 12 | 09 | 16 | 26 |
| T345A | 21 (1) | 123 (4) | 40 (9) | 56 (1) | 33 (4) | 103 (9) |
| R346A | 1 (0) | 93 (1) | 81 (7) | 145 (4) | 158 (8) | 106 (4) |
| S349A | 48 (3) | 60 (7) | 42 (0) | 6 (0) | 2 (0) | 52 (6) |
| T385A | 91 (4) | 101 (3) | 11 (5) | 115 (8) | 113 (4) | 97 (5) |
| N388A | 68 (1) | 74 (7) | 23 (8) | 74 (3) | 58 (6) | 72 (6) |
| F392A | 86 (0) | 103 (5) | 7 (3) | 112 (2) | 114 (1) | 93 (1) |
| K444A | 2 (1) | 102 (6) | 135 (5) | 91 (1) | 96 (5) | 105 (4) |
| G446A | 7 (0) | 82 (3) | 74 (0) | 2 (1) | 1 (0) | 56 (2) |
| G447A | 1 (0) | 97 (1) | 66 (3) | 0 (0) | 1 (0) | 85 (13) |
| N448A | 21 (0) | 49 (2) | 55 (6) | 0 (0) | 0 (0) | 31 (3) |
| Y449A | 0 (0) | 97 (3) | 158 (7) | 0 (0) | 1 (1) | 72 (3) |
| N450A | 19 (1) | 85 (2) | 220 (36) | 1 (0) | 1 (0) | 71 (7) |
| L452A | 112 (0) | 115 (1) | 134 (3) | 0 (0) | 0 (0) | 103 (2) |
| F456A | 85 (1) | 21 (2) | 135 (4) | 74 (0) | 66 (2) | 45 (2) |
| A475S | 77 (4) | 8 (1) | 106 (0) | 93 (7) | 85 (0) | 94 (9) |
| E484A | 56 (1) | 2 (0) | 93 (4) | 5 (0) | 1 (0) | 105 (2) |
| G485A | 74 (4) | 62 (1) | 70 (3) | 106 (0) | 77 (3) | 15 (9) |
| F486A | 108 (2) | 1 (0) | 168 (2) | 113 (2) | 106 (2) | 12 (4) |
| N487A | 66 (0) | 61 (1) | 55 (2) | 93 (1) | 74 (0) | 25 (2) |
| Y489A | 74 (1) | 1 (0) | 91 (19) | 52 (6) | 52 (3) | 10 (6) |
| F490A | 86 (2) | 75 (1) | 123 (12) | 0 (0) | 1 (0) | 109 (0) |
| G496A | 88 (12) | 102 (9) | 143 (10) | 12 (1) | 4 (0) | 104 (1) |

The mAb reactivity for each alanine scan mutant are expressed as percent of binding to wildtype S protein, with ranges (half of the maximum minus minimum values) in parentheses. At least two replicate values were obtained for each experiment. Values are underlined for critical residues.

TABLE 7
Primers for gene cloning, virus sequencing, virus construction

| | SEQ | | SEQ | |
|---|---|---|---|---|
| | ID | | ID | |
| | NO | | NO | |
| Primers for | | | | |
| constructing RBD | | | | |
| expressing vectors | | Forward primer | | Reverse primer |
| SCOV2-RBD | 347 | ACAGGTGTCCACTCGCTAGCT | 364 | GTGAACCGCCTCCACCTGCG |
| | | AGAGTGCAGCCTACCGAGA | | CAGAAGTTCACGCATTTGTT |
| K444R overlap | 348 | AGCAACAACCTGGACAGCCGG | 365 | GTTGCCGCCGACCCGGCTGT |
| | | GTCGGCGGCAAC | | CCAGGTTGTTGCT |
| K444S overlap | 349 | AGCAACAACCTGGACAGCTCC | 366 | GTTGCCGCCGACGGAGCTGT |
| | | GTCGGCGGCAAC | | CCAGGTTGTTGCT |
| E484A overlap | 350 | ACCCCTTGCAATGGCGTGGCC | 367 | GAAGTAGCAGTTGAAGCCGG |
| | | GGCTTCAACTGCTACTTC | | CCACGCCATTGCAAGGGGT |
| F486S overlap | 351 | TGCAATGGCGTGGAAGGCTCC | 368 | CAGTGGGAAGTAGCAGTTGG |
| | | AACTGCTACTTCCCACTG | | AGCCTTCCACGCCATTGCA |
| N501A overlap | 352 | TACGGCTTCCAGCCTACAGCC | 369 | AGGCTGGTAGCCCACGCCGG |
| | | GGCGTGGGCTACCAGCCT | | CTGTAGGCTGGAAGCCGTA |
| Primers for scFv | | | | |
| sequencing | | Forward primer | | |
| PSCFVF | 353 | GAAATACCTGCTGCCGACTG | | |
| Primers for virus | | | | |
| sequencing | | Forward primer | | Reverse primer |
| cov-21521V | 354 | tgttatttctagtgatgttct | | |
| | | tg | | |
| cov-22092V | 355 | tggaccttgaaggaaaac | | |
| cov-22685V | 356 | tccacttttaagtgttatgga | | |
| | | g | | |
| cov-23203V | 357 | aggcacaggtgttcttac | | |
| cov-23840V | 358 | gtacacaattaaaccgtgc | | |
| cov-24428V | 359 | cacaagctttaaacacgc | | |
| cov-25068V | 360 | tctctggcattaatgcttc | | |
| cov-25238R | 361 | | 370 | CAATCAAGCCAGCTATAAAA |
| | | | | CC |
| Primers for virus | | | | |
| construction | | Forward primer | | Reverse primer |
| K444R SARS- | 362 | ctaacaatcttgattctaGgg | 371 | caccaaccCtagaatcaaga |
| CoV-2 | | ttggtggtaattataattac | | ttgttag |
| E484A SARS- | 363 | gttgCaggttttaattgttac | 372 | ggaaagtaacaattaaaacc |
| CoV-2 | | tttcctttac | | tGcaacaccattacaaggtg |

All of the methods disclosed and claimed herein can be made and executed without undue experimentation in light of the present disclosure. While the compositions and methods provided in this disclosure have been described in terms of certain embodiments, it will be apparent to those of skill in the art that variations can be applied to the methods and in the steps or in the sequence of steps of the method described herein without departing from the concept, spirit and scope of the disclosure. More specifically, it will be apparent that certain agents which are both chemically and physiologically related can be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Ko et al., Challenges of convalescent plasma infusion therapy in Middle East respiratory coronavirus infection: a single centre experience. Antiviral therapy 2018, 23, 617-22.
Sanders, J. M., Monogue, M. L., Jodlowski, T. Z. & Cutrell, J. B. Pharmacologic Treatments for Coronavirus Disease 2019 (COVID-19): A Review. Jama, doi:10.1001/jama.2020.6019 (2020).
Thanh Le, T. et al. The COVID-19 vaccine development landscape. Nature reviews. Drug discovery 19, 305-306, doi:10.1038/d41573-020-00073-5 (2020).
Hoffmann, M. et al. SARS-CoV-2 Cell Entry Depends on ACE2 and TMPRSS2 and Is Blocked by a Clinically Proven Protease Inhibitor. Cell 181, 271-280 e278, doi:10.1016/j.cell.2020.02.052 (2020).
Li, F. Structure, Function, and Evolution of Coronavirus Spike Proteins. Annual review of virology 3, 237-261, doi:10.1146/annurev-virology-110615-042301 (2016).
Wrapp, D. et al. Cryo-EM structure of the 2019-nCoV spike in the prefusion conformation. Science 367, 1260-1263, doi:10.1126/science.abb2507 (2020).
Gao, Q. et al. Rapid development of an inactivated vaccine candidate for SARS-CoV-2. Science, doi:10.1126/science.abc1932 (2020).
Premkumar, L. et al. The receptor binding domain of the viral spike protein is an immunodominant and highly specific target of antibodies in SARS-CoV-2 patients. Science immunology 5, doi:10.1126/sciimmunol.abc8413 (2020).
Shang, J. et al. Structural basis of receptor recognition by SARS-CoV-2. Nature 581, 221-224, doi:10.1038/s41586-020-2179-y (2020).
Cao, Y. et al. Potent neutralizing antibodies against SARS-CoV-2 identified by high-throughput single-cell sequencing of convalescent patients' B cells. Cell, doi:10.1016/j.cell.2020.05.025 (2020).
Seydoux, E. et al. Analysis of a SARS-CoV-2-Infected Individual Reveals Development of Potent Neutralizing Antibodies with Limited Somatic Mutation. Immunity 53, 98-105 e105, doi:10.1016/j.immuni.2020.06.001 (2020).
Zost, S. J. et al. Rapid isolation and profiling of a diverse panel of human monoclonal antibodies targeting the SARS-CoV-2 spike protein. Nature medicine 26, 1422-1427, doi:10.1038/s41591-020-0998-x (2020a).
Ju, B. et al. Human neutralizing antibodies elicited by SARS-CoV-2 infection. Nature, doi:10.1038/s41586-020-2380-z (2020).
Robbiani, D. F. et al. Convergent antibody responses to SARS-CoV-2 in convalescent individuals. Nature 584, 437-442, doi:10.1038/s41586-020-2456-9 (2020).
Shi, R. et al. A human neutralizing antibody targets the receptor binding site of SARS-CoV-2. Nature, doi:10.1038/s41586-020-2381-y (2020).
Zost, S. J. et al. Potently neutralizing and protective human antibodies against SARS-CoV-2. Nature, doi:10.1038/s41586-020-2548-6 (2020b).
Hansen, J. et al. Studies in humanized mice and convalescent humans yield a SARS-CoV-2 antibody cocktail. Science, doi:10.1126/science.abd0827 (2020).
Rogers, T. F. et al. Isolation of potent SARS-CoV-2 neutralizing antibodies and protection from disease in a small animal model. Science, doi:10.1126/science.abc7520 (2020).
Wrapp, D. et al. Structural Basis for Potent Neutralization of Betacoronaviruses by Single-Domain Camelid Antibodies. Cell 181, 1436-1441, doi: 10.1016/j.cell.2020.05.047 (2020).
Paul-Pletzer K: Tocilizumab: blockade of interleukin-6 signaling pathway as a therapeutic strategy for inflammatory disorders. Drugs Today (B arc) 2006, 42:559-76.
Slater H: FDA Approves Phase III Clinical Trial of Tocilizumab for COVID-19 Pneumonia. world-wide-webe at cancernetwork.com/news/fda-approves-phase-iii-clinical-trial-tocilizumab-covid-19-pneumonia, 2020.
Pinto, D. et al. Cross-neutralization of SARS-CoV-2 by a human monoclonal SARS-CoV antibody. Nature, doi:10.1038/s41586-020-2349-y (2020).
Lv, Z. et al. Structural basis for neutralization of SARS-CoV-2 and SARS-CoV by a potent therapeutic antibody. Science, doi:10.1126/science.abc5881 (2020).
Junxian Ou, Z. Z., Ruixue Dai, Jing Zhang, Wendong Lan, Shan Zhao, Jianguo Wu, Donald Seto, Lilian Cui, Gong Zhang, Qiwei Zhang Emergence of RBD mutations in circulating SARS-CoV-2 strains enhancing the structural stability and human ACE2 receptor affinity of the spike protein. bioRxiv, doi:doi: //doi.org/10.1101/2020.03.15.991844 (2020).
Starr, T. N. et al. Deep Mutational Scanning of SARS-CoV-2 Receptor Binding Domain Reveals Constraints on Folding and ACE2 Binding. Cell 182, 1295-1310 e1220, doi: 10.1016/j.cell.2020.08.012 (2020).
Yi, C. et al. Key residues of the receptor binding motif in the spike protein of SARS-CoV-2 that interact with ACE2 and neutralizing antibodies. Cellular & molecular immunology, doi:10.1038/s41423-020-0458-z (2020).
Baum, A. et al. Antibody cocktail to SARS-CoV-2 spike protein prevents rapid mutational escape seen with individual antibodies. Science, doi:10.1126/science.abd0831 (2020).
Wang, L. et al. Importance of Neutralizing Monoclonal Antibodies Targeting Multiple Antigenic Sites on the Middle East Respiratory Syndrome Coronavirus Spike Glycoprotein To Avoid Neutralization Escape. Journal of virology 92, doi:10.1128/JVI.02002-17 (2018).
ter Meulen, J. et al. Human monoclonal antibody combination against SARS coronavirus: synergy and coverage of escape mutants. PLoS medicine 3, e237, doi:10.1371/journal.pmed.0030237 (2006).
Levine, M. M. Monoclonal Antibody Therapy for Ebola Virus Disease. The New England journal of medicine 381, 2365-2366, doi:10.1056/NEJMe1915350 (2019).
Du, S. et al. Structurally Resolved SARS-CoV-2 Antibody Shows High Efficacy in Severely Infected Hamsters and Provides a Potent Cocktail Pairing Strategy. Cell, doi: 10.1016/j.cell.2020.09.035 (2020).
Wu, Y. et al. A noncompeting pair of human neutralizing antibodies block COVID-19 virus binding to its receptor ACE2. Science, doi:10.1126/science.abc2241 (2020).
Xie, X. et al. An Infectious cDNA Clone of SARS-CoV-2. Cell host & microbe 27, 841-848 e843, doi: 10.1016/j.chom.2020.04.004 (2020).
Wang, Q. et al. Structural and Functional Basis of SARS-CoV-2 Entry by Using Human ACE2. Cell 181, 894-904 e899, doi:10.1016/j.cell.2020.03.045 (2020).
Yuan, M. et al. Structural basis of a shared antibody response to SARS-CoV-2. Science, doi:10.1126/science.abd2321 (2020a).
Yuan, M. et al. A highly conserved cryptic epitope in the receptor binding domains of SARS-CoV-2 and SARS-CoV. Science 368, 630-633, doi:10.1126/science.abb7269 (2020b).
Zhang, N. N. et al. A Thermostable mRNA Vaccine against COVID-19. Cell 182, 1271-1283 e1216, doi:10.1016/j.cell.2020.07.024 (2020).
Dinnon, K. H., 3rd et al. A mouse-adapted model of SARS-CoV-2 to test COVID-19 countermeasures. Nature 586, 560-566, doi:10.1038/s41586-020-2708-8 (2020).
Gu, H. et al. Adaptation of SARS-CoV-2 in BALB/c mice for testing vaccine efficacy. Science, doi:10.1126/science.abc4730 (2020).
Tyler N. Starr, A. J. G., Sarah K. Hilton, Katharine H. D. Crawford, Mary Jane Navarro, John E. Bowen, M. Alejandra Tortorici, Alexandra C. Walls, David Veesler, Jesse D. Bloom. Deep mutational scanning of SARS-CoV-2 receptor binding domain reveals constraints on folding and ACE2 binding. bioRxiv, //doi.org/10.1101/2020.06.17.157982 (2020).
Zhao, S. et al. Partial Leptin Reduction as an Insulin Sensitization and Weight Loss Strategy. Cell metabolism 30, 706-719 e706, doi:10.1016/j.cmet.2019.08.005 (2019).
Chou, T. C. & Talalay, P. Quantitative analysis of dose-effect relationships: the combined effects of multiple drugs or enzyme inhibitors. Advances in enzyme regulation 22, 27-doi:10.1016/0065-2571(84)90007-4 (1984).
Davidson, E. & Doranz, B. J. A high-throughput shotgun mutagenesis approach to mapping B-cell antibody epitopes. Immunology 143, 13-20, doi:10.1111/imm 12323 (2014).
Min, X. J., Butler, G., Storms, R. & Tsang, A. OrfPredictor: predicting protein-coding regions in EST-derived sequences. Nucleic acids research 33, W677-680, doi:10.1093/nar/gki394 (2005).
Sievers, F. et al. Fast, scalable generation of high-quality protein multiple sequence alignments using Clustal Omega. Molecular systems biology 7, 539, doi:10.1038/msb.2011.75 (2011).

1. An isolate isolated monoclonal antibody or an antigen-binding fragment thereof comprising a heavy chain variable region (VH) and a light chain variable region (VL) comprising six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3, comprise, respectively, the amino acid sequences SEQ ID NO: 31, SEQ ID NO: 32, SEQ ID NO: 33, SEQ ID NO: 34, SEQ ID NO: 35, and SEQ ID NO: 36; SEQ ID NO: 43, SEQ ID NO: 44, SEQ ID NO: 45, SEQ ID NO: 46, SEQ ID NO: 47, and SEQ ID NO: 48; SEQ ID NO: 55, SEQ ID NO: 56, SEQ ID NO: 57, SEQ ID NO: 58, SEQ ID NO: 59, and SEQ ID NO: 60; SEQ ID NO: 67, SEQ ID NO: 68, SEQ ID NO: 69, SEQ ID NO: 70, SEQ ID NO: 71, and SEQ ID NO: 72; SEQ ID NO: 79, SEQ ID NO: 80, SEQ ID NO: 81, SEQ ID NO: 82, SEQ ID NO: 83, and SEQ ID NO: 84; SEQ ID NO: 133, SEQ ID NO: 134, SEQ ID NO: 135, SEQ ID NO: 136, SEQ ID NO: 137, and SEQ ID NO: 138; SEQ ID NO: 187, SEQ ID NO: 188, SEQ ID NO: 189, SEQ ID NO: 190, SEQ ID NO: 191, and SEQ ID NO: 192; SEQ ID NO: 193, SEQ ID NO: 194, SEQ ID NO: 195, SEQ ID NO: 196, SEQ ID NO: 197, and SEQ ID NO: 198; SEQ ID NO: 199, SEQ ID NO: 200, SEQ ID NO: 201, SEQ ID NO: 202, SEQ ID NO: 203, and SEQ ID NO: 204; SEQ ID NO: 205, SEQ ID NO: 206, SEQ ID NO: 207, SEQ ID NO: 208, SEQ ID NO: 209, and SEQ ID NO: 210; SEQ ID NO: 211, SEQ ID NO: 212, SEQ ID NO: 213, SEQ ID NO: 214, SEQ ID NO: 215, and SEQ ID NO: 216; or SEQ ID NO: 217, SEQ ID NO: 218, SEQ ID NO: 219, SEQ ID NO: 220, SEQ ID NO: 221, and SEQ ID NO: 222.

2. The isolated monoclonal antibody or an antigen-binding fragment thereof of claim 1, wherein the HCDR1, HCDR2, HCDR3, LCDR1, LCDR2, and LCDR3, comprise, respectively, the amino acid sequences SEQ ID NO: 67, SEQ ID NO: 68, SEQ ID NO: 69, SEQ ID NO: 70, SEQ ID NO: 71, and SEQ ID NO: 72.

3. The isolated monoclonal antibody or an antigen-binding fragment thereof of claim 1, wherein the HCDR1, HCDR2, HCDR3, LCDR1, LCDR2, and LCDR3, comprise, respectively, the amino acid sequences SEQ ID NO: 205, SEQ ID NO: 206, SEQ ID NO: 207, SEQ ID NO: 208, SEQ ID NO: 209, and SEQ ID NO: 210.

4. The isolated monoclonal antibody or an antigen-binding fragment thereof of claim 1, wherein the VH and VL comprise amino acid sequences having at least 80%, 90%, or 95% identity to the amino acid sequences SEQ ID NO: 233 and SEQ ID NO: 234, SEQ ID NO: 237 and SEQ ID NO: 238, SEQ ID NO: 241 and SEQ ID NO: 242, SEQ ID NO: 245 and SEQ ID NO: 246, SEQ ID NO: 249 and SEQ ID NO: 250, or SEQ ID NO: 267 and SEQ ID NO: 268, respectively.

5. The isolated monoclonal antibody or an antigen-binding fragment thereof of claim 1, wherein the VH and VL comprise amino acid sequences having at least 80%, 90%, or 95% identity to the amino acid sequences SEQ ID NO: 245 and SEQ ID NO: 246, respectively.

6. The isolated monoclonal antibody or an antigen-binding fragment thereof of claim 1, wherein the VH and VL comprise the amino acid sequences SEQ ID NO: 233 and SEQ ID NO: 234, SEQ ID NO: 237 and SEQ ID NO: 238, SEQ ID NO: 241 and SEQ ID NO: 242, SEQ ID NO: 245 and SEQ ID NO: 246, SEQ ID NO: 249 and SEQ ID NO: 250, or SEQ ID NO: 267 and SEQ ID NO: 268, respectively.

7. The isolated monoclonal antibody or an antigen-binding fragment thereof of claim 1, wherein the VH and VL comprise the amino acid sequences SEQ ID NO: 245 and SEQ ID NO: 246, respectively.

8. The isolated monoclonal antibody or antigen binding fragment thereof of claim 1, wherein the antibody binds to the SARS-CoV-2 spike (S) protein.

9. The isolated monoclonal antibody or antigen binding fragment thereof of claim 1, wherein the antibody binds to the RBD domain (319-541) of the SARS-CoV-2 S protein.

10. The isolated monoclonal antibody or antigen binding fragment thereof of claim 1, wherein the antibody is a SARS-CoV-2 neutralizing antibody.

11. The isolated monoclonal antibody or antigen binding fragment thereof of claim 10, wherein the antibody exhibits a neutralization activity (effective concentration 50; EC50) of less than 20, 10 or 5 (μg/ml).

12. The isolated monoclonal antibody or antigen binding fragment thereof of claim 11, wherein the antibody exhibits a neutralization activity EC50 of about 0.1 to 20 (μg/ml).

13. A pharmaceutical composition comprising the isolated monoclonal antibody or an antigen-binding fragment thereof according to claim 1, and a pharmaceutically acceptable carrier.

14. An isolated nucleic acid that encodes the isolated monoclonal antibody according to claim 1.

15. A vector comprising the isolated nucleic acid of claim 14.

16. A host cell comprising the vector of claim 15.

17. A process of producing an antibody, comprising culturing the host cell of claim 16 under conditions suitable for expressing the antibody, and recovering the antibody.

18. A method of treating or ameliorating a SARS-CoV-2 infection in a subject, the method comprising administering to the subject a therapeutically effective amount of the antibody or an antigen-binding fragment thereof according to claim 1.

19. The method of claim 18, wherein the method reduces viral replication in the subject.

20. The method of claim 18, wherein the method reduces inflammation in the lungs of a subject.

21. The method of claim 18, wherein the subject has pneumonia.

22. The method of claim 18, wherein the subject is on a respirator or oxygen supplementation.

23. The method of claim 18, wherein the antibody or an antigen-binding fragment thereof is administered intravenously, intra-arterially, subcutaneously or via inhalation.
